(19) United States
(12) Patent Application Publication
(10) Pub. No.: US 2013/0150560 A1
(43) Pub. Date: Jun. 13, 2013

(54) AMYLOID BETA-DERIVED DIFFUSIBLE LIGANDS (ADDLs), ADDL-SURROGATES, ADDL-BINDING MOLECULES, AND USES THEREOF

(71) Applicant: NORTHWESTERN UNIVERSITY, Evanston, IL (US)

(72) Inventors: Grant A. Krafft, Glenview, IL (US); William L. Klein, Winetka, IL (US); Brett A. Chromy, Danville, CA (US); Lei Chang, Westmont, IL (US); Mary P. Lambert, San Antonio, TX (US); Kirsten L. Viola, Chicago, IL (US)

(73) Assignee: NORTHWESTERN UNIVERSITY, Evanston, IL (US)

(21) Appl. No.: 13/676,806

(22) Filed: Nov. 14, 2012

Related U.S. Application Data
(63) Continuation of application No. 11/142,869, filed on Jun. 1, 2005.
Application No. 11/142,869 is a continuation of application No. 10/924,372, filed on Aug. 23, 2004, now abandoned.
Application No. 10/924,372 is a continuation of application No. 10/676,871, filed on Oct. 1, 2003, now abandoned.
(60) Provisional application No. 60/415,074, filed on Oct. 1, 2002.

(51) Int. Cl. C07K 16/18

(52) U.S. Cl. CPC C07K 16/18
USPC 530/389.1; 435/7.92

(57) ABSTRACT

The invention herein comprises amyloid beta-derived diffusible ligands (ADDLs), compositions comprising ADDLs, ADDL-surrogates, ADDL-binding molecules, and methods of using any of the foregoing compounds and compositions. ADDLs comprise amyloid β protein assembled into soluble, globular, non-fibrillar, oligomeric structures capable of activating specific cellular processes. The invention also comprises methods of generating ADDL-specific antibodies and methods of using ADDL-specific antibodies for assaying the formation, presence, receptor protein binding and cellular activity of ADDLs, as well as using such antibodies to detect compounds that block the formation or activity of ADDLs, and methods of identifying such compounds. The invention further provides methods of using ADDL-specific antibodies in modulating ADDL formation and/or activity, inter alia in the treatment of learning and/or memory disorders.

### CROSS REFERENCE TO RELATED APPLICATIONS

This application is a continuation of U.S. patent application Ser. No. 11/142,869 filed Jun. 1, 2005, which is a continuation of U.S. patent application Ser. No. 10/924,372, filed Aug. 23, 2004, now abandoned, which is a continuation of U.S. patent application Ser. No. 10/676,871, filed Oct. 1, 2003, now abandoned, which claims priority from U.S. patent application No. 60/415,074, filed Oct. 1, 2002.

### STATEMENT REGARDING FEDERALLY SPONSORED RESEARCH AND/OR DEVELOPMENT

The invention was made, in part, with support from agencies of the United States government. Accordingly, the government has certain rights in the invention.

### REFERENCE TO AN APPENDIX SUBMITTED ON COMPACT DISK

Not Applicable

### BACKGROUND OF THE INVENTION

(1) Field of the Invention

The invention relates to the fields of medicine, biology, biochemistry, molecular biology and cellular biology. In particular, the invention relates to degenerative neurological disorders. More in particular, the invention relates to the diagnosis and treatment of degenerative neurological disorders. Even more in particular, the invention relates to compositions comprising amyloid beta (Aβ)-derived diffusible ligands (ADDLs), ADDL receptor(s), and antibodies to ADDLs and/or ADDL receptors. The invention further relates to the use of ADDLs, ADDL receptors, and/or antibodies to ADDLs and/or ADDL receptors in the diagnosis and/or treatment of degenerative neurological disorders.

(2) Description of the Related Art

The invention is related to the invention disclosed in U.S. patent application Ser. No. 10/166,856, filed 11 Jun. 2002, which is a continuation-in-part of U.S. patent application Ser. No. 09/369,236, filed 4 Aug. 1999, which is a continuation-in-part of U.S. patent application Ser. No. 08/769,089, filed 5 Feb. 1997, now U.S. Pat. No. 6,218,506.

Alzheimer's disease (AD) is the most common cause of dementia in older individuals. No effective treatment exists, however significant research progress has led to a general consensus that elevated levels of Aβ₁₋₄₂, the longer form of the amyloid beta (Aβ) peptide, are responsible for the disease. Exactly how such elevated levels of Aβ₁₋₄₂ lead to the disease has not been precisely elucidated, but the most frequently invoked and longstanding explanation is the amyloid cascade hypothesis involving deposition of amyloid fibrils and the purported toxic activity thereof (Hardy, J. A. & Higgins, G. A. (1992) Science, vol. 256, pp. 184-185; Small, D. H. (1998) Amyloid, vol. 5, pp. 301-304; Golde, T. E. (2000) Biochim. Biophys. Acta, vol. 1502, pp. 172-187). Other published studies claim that multiple factors are involved, including CNS inflammation, oxidative damage, and cytoskeletal anomalies (McGeer, P. L. & McGeer, E. G. (1999) J. Leukoc. Biol., vol. 65, pp. 409-415; Mandelkow, E. M. & Mandelkow, E. (1998) Trends Cell Biol., vol. 8, pp. 425-427; Spillantini, M. G. & Goedert, M. (1998) Trends Neurosci., vol. 21, pp. 428-433; Smith, M. A. et al. (1995) Trends Neurosci., vol. 18, pp. 172-176), but these phenomena have been argued to be caused by elevated Aβ₁₋₄₂ levels, and not themselves the root cause of the disease.

Aβ₁₋₄₂ is a 42-amino acid amphipathic peptide derived proteolytically from a widely expressed membrane precursor protein (Selkoe, D. J. (1994) Annu. Rev. Neurosci., vol. 17, pp. 489-517). As a monomer, the amyloid peptide has never been demonstrated to have toxic effects, and in some studies it has been purported to have neurotrophic effects. Native Aβ1-42 has the sequence: DAEFRHDSGYEVHHQKLVFFAEDVGSNKGAIIGLMVGGVVIA.

Monomers of Aβ₁₋₄₂ assemble into at least three neurotoxic species: fibrillar amyloid (Pike, C. J. et al. (1993) J. Neurosci., vol. 13, pp. 1676-1687; Lorenzo, A. & Yanker, B. A. (1994) Proc. Natl. Acad. Sci. USA, vol. 91, pp. 12243-12247), protofibrils (Hartley, D. M. et al. (1999) J. Neurosci., vol. 19, pp. 8876-8884; Walsh, D. M. et al. (1999) J. Biol. Chem., vol. to 274, pp. 25945-25952, and Aβ₁₋₄₂-derived diffusible ligands (ADDLs) (Lambert, M. P. et al. (1998) Proc. Natl. Acad. Sci. USA, vol. 95, pp. 6448-6453). Fibrillar amyloid is insoluble, and deposits of fibrillar amyloid are easily detected in AD and transgenic mice because of their birefringence with dyes such as thioflavin S. Fibrillar amyloid is a major protein component of senile plaques in Alzheimer's disease brain. Aβ peptides of various lengths, including Aβ1-40, 1-42, 1-43, 25-35, and 1-28 assemble into fibrils in vitro. All of these fibrils have been reported to be toxic to neurons in vitro and to activate a broad range of cellular processes. Hundreds of studies describe Aβ fibril neurotoxicity, but numerous studies also describe poor reproducibility and highly variable toxicity results. The variability has been attributed, in part, to batch-to-batch differences in the starting solid peptide and these differences relate specifically to the various physical or aggregation states of the peptide, rather than the chemical structure or composition. Protofibrils are large yet soluble meta-stable structures first identified as intermediates en route to full-sized amyloid fibrils (Walsh, D. M. et al. (1997) J. Biol. Chem., vol. 272, pp. 22364-22372).

ADDLs (amyloid beta (Aβ)-derived diffusible ligands) comprise small, soluble Aβ₁₋₄₂ oligomers, predominantly trimers and tetramers but also higher-order species (Lambert, M. P. et al. (1998) Proc. Natl. Acad. Sci. USA, vol. 95, pp. 6448-6453; Chromy, B. A. et al. (2000) Soc. Neurosci. Abstr., vol. 26, p. 1284). All three forms of assembled Aβ₁₋₄₂ rapidly impair reduction of the dye MTT (Shearman, M. S. et al. (1994) Proc. Natl. Acad. Sci. USA, vol. 91, pp. 1470-1474; Walsh, D. M. et al. (1999) J. Bio. Chem., vol. 274, pp. 25945-25952; Oda, T. et al. (1995) Exp. Neurol., vol. 136, pp. 22-31), possibly the consequence of impaired vesicle trafficking (Liu, Y. & Schubert, D. (1997) J. Neurochem., vol. 69, pp. 2285-2293), and they ultimately kill neurons (Longo, V. D. et al. (2000) J. Neurochem., vol. 75, pp. 1977-1985; Loo, D. T. et al. (1993) Proc. Natl. Acad. Sci. USA, vol. 90, pp. 7951-7955; Hartley, D. M. et al. (1999) J. Neurosci., vol. 19, pp. 8876-8884). All three forms also exhibit very fast electrophysiological effects. Amyloid and protofibrils broadly disrupt neuronal membrane properties, inducing membrane depolarization, action potentials, and increased EPSPs (Hartley, D. M. et al. (1999) J. Neurosci., vol. 19, pp. 8876-8884), while ADDLs selectively block long-term potentiation (LTP) (Lambert, M. P. et al. (1998) Proc. Natl. Acad. Sci. USA, vol. 95, pp. 6448-6453; Wang, H. et al. (2000) Soc. Neurosci. Abstr., vol. 26, pp. 1787; Wang et al. (2002), Brain Research 924, 133-140). ADDLs also show selectivity in neurotoxicity, killing hippocampal but not cerebellar neurons in brain slice cultures (Kim, H.-J. (2000) Doctoral Thesis, Northwestern University, pp. 1-169). Given the poor correlation between fibrillar amyloid and disease progression (Terry, R. D. (1999) in Alzheimer's Disease (Terry, R. D. et al., Eds.), pp. 187-206, Lippincott Williams & Wilkins), it is likely that fibrillar amyloid deposits are not the toxic form of Aβ₁₋₄₂ most relevant to AD. Non-fibrillar assemblies of Aβ occur in AD brains (Kuo, Y. M. et al. (1996) J. Biol. Chem., vol. 271, pp. 4077-4081; Roher, A. E. et al. (1996) J. Biol. Chem., vol. 271, pp. 20631-20635; Enya, M. et al. (1999) Am. J. Pathol., vol. 154, pp. 271-279; Funato, H. et al. (1999) Am. J. Pathol., vol. 155, pp. 23-28; Pitschke, M. et al. (1998) Nature Med., vol. 4, pp. 832-834) and these species appear to correlate better than amyloid with the severity of AD (McLean, C. A. et al. (1999) Ann. Neurol., vol. 46, pp. 860-866; Lue, L. F. et al. (1999) Am. J. Pathol., vol. 155, pp. 853-862). Soluble Aβ oligomers are likely to be responsible for neurological deficits seen in multiple strains of transgenic mice that do not produce amyloid plaques (Mucke, L. et al. (2000) J. Neurosci., vol. 20, pp. 4050-4058; Hsia, A. Y. et al. (1999) Proc. Natl. Acad. Sci. USA, vol. 96, pp. 3228-3233; Klein, W. L. (2000) in Molecular Mechanisms of Neurodegenerative Diseases (Chesselet, M.-F., Ed.), Humana Press; Klein, W. L. et al. (2001) Trends Neurosci., vol. 24, pp. 219-224).

Over the past 3 years, a novel therapeutic strategy for Alzheimer's disease has emerged, based on vaccination with aggregated Aβ preparations. The initial studies that utilized this approach involved transgenic AD model mice that were vaccinated with Aβ fibrils, a procedure which was reported to afford some protection from behavioral deficits normally manifest in these mice (Schenk, D. (1999) Nature, vol. 400, pp. 173-177; Morgan D. G. et al. (2001) Nature, in press; Helmuth, L. (2000) Science, vol. 289, p. 375; Arendash, G. et al. (2000) Soc. Neurosci. Abstr., vol. 26, p. 1059; Yu, W. et al. (2000) Soc. Neurosci. Abstr., vol. 26, p. 497). This result was surprising because it had generally not been appreciated that effective immune protection could be conferred on the brain side of the blood brain barrier (BBB). Apparently the protective effects observed in these transgenic AD mouse vaccination studies resulted from direct transport of anti-amyloid antibodies across the blood brain barrier in sufficient quantities to reduce the levels of toxic amyloid structures. Alternatively, it is conceivable that antibodies circulating in the bloodstream were capable of binding and clearing amyloid in sufficient quantities to reduce brain levels and produce a beneficial symptomatic effect. Several of the Tg mouse vaccination studies reported that total brain amyloid levels had not been lowered significantly, compared with amyloid levels in unvaccinated Tg AD mice in the control groups, which raises doubts about the plausibility of the Aβ clearance mechanism.

In other studies, it was demonstrated that direct injection of anti-amyloid antibodies into the brains of transgenic AD mice resulted in a significant reduction in brain amyloid levels (Bard, F. et al. (2000) Nature Med., vol. 6, pp. 916-919), however this approach involved delivery of antibody levels significantly higher than could be expected from passive transport across the BBB.

Regardless of the operative mechanism in these vaccinated Tg AD mice, the promising behavioral protection results provided ample impetus to move forward with human testing of a fibrillar Aβ vaccine AN1792 (Helmuth, L. (2000) Science, vol. 289, p. 375). The successful Phase I safety studies led to the initiation of Phase II efficacy studies in AD patients. Unfortunately, these Phase II studies were halted recently because 12 of 97 AD patients in the study had developed vaccine related complications involving brain inflammation and encephalitis. Although the specific reason(s) for these serious complications is not known definitively, it can be surmised that vaccination with Aβ fibrils would generate a significant immune response to the amyloid plaques in the brain, and that this would result in persistent activation of microglial cells and production of inflammatory mediators, all of which would contribute to severe encephalitis. In fact, this glial activation mechanism is precisely the mechanism proposed to explain the efficacy of this vaccine approach (Schenk, D. (1999) Nature, vol. 400, pp. 173-177).

These results now make it very clear that any successful immune strategy for prevention or therapy of AD, whether involving a vaccine or a therapeutic antibody, will require a much more selective approach that targets toxic structures directly and specifically.

Previous immunization protocols (e.g., the AN1792 protocol discussed above) have used aggregated solutions of Aβ₁₋₄₂ that contain multiple forms of Aβ₁₋₄₂ in undefined proportions.

Thus, a need exists for solutions to the problems that have plagued the art to this point. The invention described herein is based on the use of well-defined ADDL preparations consisting of Aβ₁₋₄₂ monomers and small oligomers, injected at low doses. The data presented herein show that Aβ₁₋₄₂ oligomers are more potent immunogens than Aβ monomer, giving rise to antibodies that preferentially recognize ADDLs in immunoblots, detect puncta of ADDLs bound to cell surfaces in immunohistochemistry protocols, and block the toxic action of ADDLs on cultured PC12 cells. These results support the hypothesis that therapeutic antibodies targeting small non-fibrillar Aβ₁₋₄₂ toxins can be effective agents to diagnose and treat, either prophylactically and/or therapeutically, AD pathogenesis.

### BRIEF SUMMARY OF THE INVENTION

One aspect of the present invention provides an immune strategy for prophylactic and/or therapeutic treatment of AD, wherein the treatment comprises a selective approach that targets toxic structures directly and specifically. The approach can be independent of amyloid clearance, whether fibrillar or monomeric. The present invention provides an immune strategy that directly targets and neutralizes ADDLs.

Another aspect of the invention provides antibodies that have been generated and selected for the ability to bind ADDLs specifically, without binding to Aβ monomer or amyloid fibrils. Such antibodies can be employed to treat and prevent disease that results from the action of ADDLs in the brain.

Still another aspect of the invention provides anti-ADDL antibodies for specific diagnosis of individuals who have measurable levels of ADDLs present in the serum, brain or CSF.

An additional aspect of the invention provides anti-ADDL antibodies for use in assays that allow for the detection of molecules that block the formation or activity of ADDLs.

The present invention seeks to overcome the substantial problems with the prior art that are based largely on the flawed theory that amyloid fibrils and plaques cause AD. Accordingly, one object of the present invention is the production, characterization and use of new compositions comprising specific ADDL-binding molecules such as anti-ADDL antibodies, which are capable of direct or indirect interference with the activity and/or formation of ADDLs (soluble, globular, non-fibrillar oligomeric Aβ₁₋₄₂ assemblies). These and other objects and advantages of the present invention, as well as additional inventive features, will be apparent from the description herein.

The present invention pertains to amyloid beta-derived diffusible ligands (ADDLs), antibodies that bind to ADDLs (anti-ADDL antibodies), uses of anti-ADDL antibodies to discover anti-ADDL therapeutics, and uses of anti-ADDL antibodies in the diagnosis, treatment and prevention of diseases associated with ADDLs, including Alzheimer's disease, learning and memory disorders, and neurodegenerative disorders. The invention specifically pertains to antibodies that recognize and bind ADDLs preferentially, with much lower binding capability for monomer forms of the amyloid peptide. Antibodies with these characteristics are useful for blocking the neurotoxic activity of ADDLs, and they are useful for eliminating ADDLs from the brain via clearance of antibody-ADDL complexes. Antibodies with these characteristics also are useful for detection of ADDLs in biological samples, including human plasma, cerebrospinal fluid, and brain tissue. Anti-ADDL antibodies are useful for quantitative measurement of ADDLs in cerebrospinal fluid, enabling the diagnosis of individuals adversely affected by ADDLs. Such adverse effects may manifest as deficits in learning and memory, alterations in personality, and decline in other cognitive functions such as those functions known to be compromised in Alzheimer's disease and related disorders. Anti-ADDL antibodies are also useful for quantitative detection of ADDLs in brain tissue obtained at autopsy, to confirm pre-mortem diagnosis of Alzheimer's disease.

The invention further pertains to antibodies that recognize and bind ADDLs preferentially, with much lower binding capability for fibrillar and monomer forms of the amyloid peptide. Such antibodies are particularly useful for treatment and prevention of Alzheimer's disease and other ADDL-related diseases in patients where prevalent fibrillar amyloid deposits exist in the brain, and for whom treatment with antibodies that preferentially bind to fibrillar forms of amyloid will result in serious brain inflammation and encephalitis.

The invention further pertains to the use of ADDLs to select or identify antibodies or any other ADDL binding molecule or macromolecule capable of binding to ADDLs, clearing ADDLs from the brain, blocking ADDL activities, or preventing the formation of ADDLs. Additional inventions include new composition of matter, such molecule being capable of selecting antibodies or anti-ADDL binding molecules, or inducing an ADDL blocking immune response when administered to an animal or human. The invention extends further to include such uses when applied to methods for creating synthetic antibodies and binding molecules and other specific binding molecules through selection or recombinant engineering methods as are known in the art.

Specifically, the invention pertains to the preparation, characterization and methods of using such anti-ADDL antibodies. The invention also pertains to the use of anti-ADDL antibodies for the detection of ADDL formation and for the detection of molecules that prevent ADDL formation. The invention further pertains to the use of such antibodies to detect molecules that block ADDL binding to specific ADDL receptors present on the surface of nerve cells that are compromised in Alzheimer's disease and related disorders.

ADDLs comprise amyloid β (Aβ) peptide assembled into soluble, globular, non-fibrillar, oligomeric structures that are capable of activating specific cellular processes.

Disclosed herein are methods for preparing and characterizing antibodies specific for ADDLs as well as methods for assaying the formation, presence, receptor protein binding and cellular activities of ADDLs. Also described are compounds that block the formation or activity of ADDLs, and methods of identifying such compounds. ADDL formation and activity are relevant inter alia to compromised learning and memory, nerve cell degeneration, and the initiation and progression of Alzheimer's disease. Modulation of ADDL formation or activity thus can be employed according to the invention in the treatment of learning and memory disorders, as well as other diseases, disorders or conditions that are due to the effects of the ADDLs.

The invention pertains to new compositions of matter, termed amyloid beta-derived diffusible ligands or amyloid beta-derived dementing ligands (ADDLs). ADDLs consist of amyloid β peptide assembled into soluble non-fibrillar oligomeric structures that are capable of activating specific cellular processes. A preferred aspect of the present invention comprises antibodies and binding molecules that are specific for ADDLs, and methods for preparation, characterization and use of antibodies or binding molecules that are specific for ADDLs. Another preferred embodiment comprises antibodies or binding molecules that bind to ADDLs but do not bind to Aβ monomers or fibrillar aggregates. Another aspect of the invention consists of methods for assaying the formation, presence, receptor protein binding and cellular activities of ADDLs, and methods for diagnosing diseases or potential diseases resulting from the presence of ADDLs. A further aspect of the invention is the use of anti-ADDL antibody or anti-ADDL binding molecules for the therapy and/or prevention of Alzheimer's disease and other diseases associated with the presence of ADDLs. The invention further encompasses assay methods and methods of identifying compounds that modulate (e.g., increase or decrease) the formation and/or activity of ADDLs. Such compounds can be employed in the treatment of diseases, disorders, or conditions due to the effects of the ADDLs.

### BRIEF DESCRIPTION OF THE SEVERAL VIEWS OF THE DRAWINGS

FIG. 1 is a computer-generated image of a densitometer-scanned silver-stained polyacrylamide gel which shows the ADDLs electrophoresing with a primary band corresponding to about 30 kD, a less abundant band corresponding to about 17 kD, and no evidence of fibrils or aggregates.

FIG. 2 is a computer-generated image of a densitometer-scanned Coomassie-stained SDS-polyacrylamide gel which shows ADDLs electrophoresing with a primary band (upper doublet) corresponding to a size of about 17 to about 22 kD, and with another band (lower dark band) indicating abundant 4 kD monomer present, presumably a breakdown product. Lanes: first, molecular size markers; second ADDL preparation; third, heavier loading of ADDL preparation.

FIG. 3 is a representative computer-generated image of AFM analysis of ADDL-containing “fraction 3” (fractionated on a Superdex 75 gel filtration column).

FIG. 4 is a computer-generated image of a densitometer-scanned Coomassie-stained SDS-polyacrylamide gradient gel of ADDLs prepared by coincubation with clusterin (lane A) or cold F12 media (lane B), and of ADDLs prepared by coincubation with clusterin and which passed through a Centricon 10 kD cut-off membrane (lane C) or were retained by a Centricon 10 kD cut-off membrane (lane D). MW, molecular size markers.

FIG. 5 is a graph of ADDL concentration measured as amyloid β1-42 concentration (nM) vs. % dead cells for brain slices from mice treated with the ADDL preparations.

FIG. 6 is a bar chart showing % MTT reduction for control PC 12 cells not exposed to ADDLs (“Cont.”), PC 12 cells exposed to clusterin alone (“Apo J”), PC 12 cells exposed to monomeric Aβ (“Aβ”), PC12 cells exposed to amyloid β coaggregated with clusterin and aged one day (“Aβ:Apo J”).

FIG. 7 is a FACScan showing fluorescence intensity (0-170) versus events (0-300) for B103 cells not exposed to ADDLs (unshaded peak) and B103 cells bound to fluorescent labeled ADDLs (shaded peak).

FIG. 8 is a FACScan showing fluorescence intensity (0-200) versus events (0-300) for hippocampal cells not exposed to ADDLs (unshaded peak, “−ADDLs”) and hippocampal cells bound to fluorescent labeled ADDLs (shaded peak, “+ADDLs”).

FIG. 9 is a bar chart of percent maximum ADDL binding or ADDL-evoked death for B103 cells that either have been not exposed (“−”) or coexposed (“+”) to the peptides released by trypsinization of B 103 cells.

FIG. 10 is a graph of relative ADDL concentration vs. % dead cells for brain slices from mice treated with the ADDL preparations. To determine relative concentration, an initial concentration of 10 M Aβ protein was employed to form ADDLs at the highest data point (point “16”), this was subsequently diluted to ½ (point “8”), ¼ (point “4”), and the like.

FIG. 11 is a bar chart showing optical density obtained in the ADDL binding ELISA assay wherein B103 cells were coincubated with ADDLs and 6E10 antibody (“cells, ADDL, 6E10” bar), B103 cells were coincubated with ADDLs (“cells, ADDL” bar), B103 cells were coincubated with 6E10 antibody (“cells, 6E10” bar), B103 cells were incubated alone (“cells” bar), 6E10 antibody was incubated alone (“6E10” bar), or the optical density of diluent was read (“blank” bar).

FIG. 12 is a bar chart of % dead cells in either fyn +/+(wild type, “Fyn +”; crosshatched bars) or fyn −/− (knockout, “Fyn −”; solid bars) mice either not treated (“Medium”) or contacted with ADDLs (“ADDLs”).

FIG. 13 is a graph of Aβ concentration (1 μM) versus activated glia (number) obtained upon incubation of astrocytes with ADDLs (filled triangles) or Aβ17-42 (filled squares).

FIG. 14 is a graph of time (minutes) versus % baseline cell body spike amplitude for control mice not treated with ADDLs (filled triangles) or mice treated with ADDLs (filled squares).

FIG. 15 is a graph of time (minutes) versus mean spike amplitude for control rat hippocampal slices not exposed to ADDLs (filled triangles) versus rat hippocampal slices exposed to ADDLs (filled squares).

FIG. 16 is a computer-generated image of a densitometer-scanned 16.5% tris-tricine SDS-polyacrylamide gel (Biorad) that shows a range of oligomeric, soluble ADDLs (labeled “ADDLs”), and amyloid β dimer (labeled “Dimer”), and monomer (labeled “Monomer”). Lanes: first, silver stained Mark XII molecular weight standards (Novex, San Diego, Calif.); second, silver stained ADDLs; third, Western blot of second lane using the monoclonal antibody 26D6 (Sibia Neurosciences, San Diego, Calif.).

FIG. 17 is a computer-generated image of an AFM analysis of ADDLs. The top view subtracted image shows a high magnification view (2.0 μm×2.0 μm) of aggregated amyloid β molecules that have been spotted on freshly cleaved mica.

FIG. 18 displays data showing that ADDLs maintain their oligomeric profile and cytotoxic activity after storage at 4° C. A. Silver stain of initial ADDL preparation and the same preparation one day later. Aβ₁₋₄₂ was dissolved in DMSO, then in F12 (see Example 22, Materials and Methods), and incubated at 4° C. for 24 hours. After centrifugation, the supernatant, which represents the initial ADDL preparation, was removed to a new tube. Supernatant proteins were separated on a Tris tricine gel using SDS-PAGE and visualized with a silver stain. Lane 1: Colored molecular weight markers (not silver stained). Lane 2: Initial ADDL preparation showing abundant monomer, slight dimer, and substantial trimer and tetramer oligomers. Lane 3: ADDL preparation one day later at 4° C. showing essentially the same profile. In this image, the uniform gray background of these two lanes is from the colored background of the silver stain. B. MTT Assay of initial ADDL preparation and the same preparation one day later. The MTT assay was used to compare the effect of a 4-hour ADDL incubation on PC12 cells (Example 22, Materials and Methods). Whether fresh or stored, ADDL preparations caused at least 50% inhibition. Data from A and B indicate that the 48-hour sample, which was used for injection, is similar in structure and toxicity to the initial preparation.

FIG. 19 presents data showing that antibody M94 displays a strong preference for oligomers in immunoblots. ADDLs were separated using SDS-PAGE, transferred to nitrocellulose, and probed with the indicated antibodies. Binding was identified with a secondary conjugated to horseradish peroxidase and visualized using chemiluminescence. The monoclonal antibody 4G8 (right lane) recognizes four Aβ species, from monomer to tetramer. The monoclonals 26D6 (middle lane) and 6E10 (FIG. 3) recognize monomer, trimer, and tetramer, but not dimer. The new polyclonal antisera M94 (left lane) and M93 (FIG. 20) preferentially recognize oligomers.

FIG. 20 presents data showing that the oligomer-selective M93 antibody detects amyloid β monomer only at high antibody concentrations. A. Immunoblot: An ADDL immunoblot was probed with decreasing concentrations of antibody. Visualization of ADDLs was by chemiluminescence. M93 potency is at least that of 6E10, a commercial monoclonal antibody unselective for oligomers that is shown for reference (at a dilution of 1:2000). B. Quantification of chemiluminescent bands: The intensity of each band was determined by image analysis (Methods) and normalized to the 6E10 monomer band (100%). M93 antibody bound monomer only at higher antibody concentrations (<1:500 dilution). These data indicate that oligomers are preferentially recognized by M93 antibody.

FIG. 21 presents data showing that pre-absorption of oligomer-selective antibodies with ADDLs eliminates binding in immunoblots. Each antibody (as indicated) was incubated with ADDLs for 2 hours at 0, 1, 5, or 10 times the protein concentration. Then the solutions were used on an ADDL immunoblot that was developed in the standard manner. Prior absorption by ADDLs eliminates all binding. This result indicates that binding of the antibodies to ADDLs requires specific recognition.

FIG. 22 presents data showing that oligomer-selective antibodies exhibit no binding to normal brain proteins. In order to determine if the antibodies bind to brain proteins other than ADDLs, rat brain homogenate was prepared and separated alone or in the presence of ADDLs using SDS-PAGE. ADDLs were added to protein (60 g) immediately before electrophoresis. The resulting immunoblot was probed with M94 and binding visualized with chemiluminescence. No binding occurred to brain proteins alone (middle lane). Samples that had ADDLs and homogenate (right lane) showed tetramer and trimer (closed arrow) as well as higher molecular weight species. The most prominent of these bands are indicated by the open arrow, with trace amounts showing up at higher molecular weights. ADDLs alone are shown in the left lane. These results indicate that the antibodies recognize only Aβ oligomers and not brain proteins.

FIG. 23 presents data showing the localization of ADDL binding in cultured rat hippocampal cells. Rat hippocampal cultures were prepared, exposed to ADDLs for 90 min., and then fixed. Bound ADDLs were identified using M94 antibody and visualized with secondary IgG conjugated to Oregon green-514. The top panels are immunofluorescence images; the bottom panels are inverted fluorescent images. Left: cultures treated with ADDLs but no primary antibody. Middle: cultures treated with ADDLs and M94 antibody. Right: cultures treated with vehicle control and M94 antibody. There is no binding to primary- or ADDL-free cultures. Label seen in cultures treated with both ADDLs and M94 is located almost exclusively on neurites. The bar in the lower left corner represents 25 microns.

FIG. 24 presents data showing that toxicity to PC12 cells (as measured by an MTT assay) is blocked by ADDL-selective antibodies. Pre-immune serum was added to ADDLs for 2 hours before the MTT reaction was performed in PC12 cells. This addition does not prevent the reduction of MTT in a dose-dependent manner (open squares, bottom line). However, if antibodies are pre-incubated with ADDLs for 2 hours, no change in MTT reduction is seen (filled squares, top line). These data indicate that the antibodies block the action of ADDLs.

FIG. 25 presents data showing a selective, sensitive dot-blot assay for assembled forms of soluble Aβ. (A) An immunoblot shows that M94-3 identifies oligomers (right), while Potempska antibody (R165) identifies only monomer (left). (B) A dot blot assay showing the selectivity of M94-3 for oligomers and monomers (HFIP−) over monomers alone (HFIP+). This assay is sensitive to 10 fmol soluble Aβ₁₋₄₂. (C) The dot blot assay is linear over a 100-fold concentration range.

FIG. 26 presents data showing that assembled forms of soluble Aβ increase as much as 70-fold in Alzheimer's affected brain. (Panel A, Left) Dot blot assay of 5 AD-affected brains and 5 age-matched control brains (1 μg/dot). (Panel B, Right) Quantification of the same samples using a scatter plot.

FIG. 27 presents data showing that assembled forms of soluble Aβ in AD brain show identity with synthetic Aβ oligomers. (A) 2D immunoblot of soluble protein from AD brain. (B) 2D immunoblot of synthetic soluble oligomers ADDLs. Samples A and B contain a prominent 55 kDa protein, which is approximately the same molecular weight as an Aβ₁₋₄₂ 12-mer, with a pI of about 5.6. (C) 2D immunoblot of soluble protein from control brain.

FIG. 28 presents data showing that ADDL binding proteins are species conserved and show affinity and expression that parallels vulnerability to pathogenic ADDLs. (Top) Ligand blot using protein extracts from cerebellum (Cb), cortex (Cx), or hippocampus (Hp) of rat and human brains separated by SDS-PAGE and incubated with synthetic Aβ oligomers. The 140 kDa binding protein is more abundant in rat hippocampus and cortex than in cerebellum and is also more abundant in human cortex than in human cerebellum. (Bottom-Left) Quantitation of p140 and p260 binding proteins in ligand blots for control (CTRL) and AD (AD) brains. Both binding proteins showed lower protein levels in AD brain. (Right) Plot of soluble Aβ binding to cortex protein p140. Half maximal value is 10 nM. (Inset) MTT toxicity assay for rat cortex (left) and cerebellar (right) cultures at two ADDL concentrations. Only cortex cultures are inhibited by ADDLs.

FIG. 29 presents data showing that soluble Aβ assemblies (ADDLs) are ligands for proteins found in membrane rafts. (A) Ligand blot using rat brain membranes (three left columns) or a raft preparation (Raft) separated by SDS-PAGE and incubated with soluble human brain extracts (Extract) or synthetic ADDLs (Synth). Binding was visualized with M94-3 and chemiluminescence. Three prominent binding proteins (p260, p140, p100) were routinely observed. (B) Dot blots verified the existence of ADDLs in brain extracts, but not in control extracts.

FIG. 30 presents data showing that crosslinking of ADDLs to proteins on nerve cell surfaces reveals one protein band with a molecular weight (MW) ranging from about 280-300 kDa that is not detectable when ADDLs are not crosslinked. Cell membranes from the CNS neuroblastoma B103 line, rat brain, and rat liver are suspended in F12 medium. Different concentration of ADDLs are added on ice and shaken for 3 h in a cold room. The crosslinking agent DTSSP is added for 1 hour in the cold room. Reactions are stopped by Tris-HCl for 30 min on ice. Membrane proteins are solubilized by RIPA buffer, separated by SDS-PAGE, transferred to nitrocellulose, and processed for western blotting. One ADDL-dependent band with Mw 250-300 kDa was found in B 103 and rat brain membranes, but not in liver membranes.

FIG. 31 presents data showing 2D gel electrophoresis of membrane proteins (Panel A, left—silver stain) reveals p260 by ligand blotting with ADDLs and ADDL specific antibody (Panel B, right). For this assay, about 150 g of cortex membrane proteins were separated by 2-D gel electrophoresis, transferred to a nitrocellulose membrane, incubated with 10 nM ADDLs in cold room for 3 h, then detected by ADDLs—M94-3 ADDL-selective antibody and electrochemiluminescent visualization. Panel A) Silver stain. Panel B) ADDL—Far western blot. A single 2D separation provides pure p260, and reveals that p260 is a non-abundant protein with a pI of about 5.6.

FIG. 32 presents data showing that soluble assemblies of Aβ (ADDLs) bind to neuronal receptor proteins with punctate distribution (i.e., ADDLs are ligands for neuronal cell surface proteins). ADDL (oligomer) binding is visualized by immunofluorescence microscopy using the anti-ADDL antibody M94-3. (A) Soluble AD-brain proteins bound to cultured hippocampal nerve cells. (B) Soluble control-brain proteins bound to cultured hippocampal nerve cells. (C) Synthetic ADDLs prepared from Aβ₁₋₄₂ bound to cultured hippocampal nerve cells. ADDL distribution is punctate and small (˜0.2-0.5 μm). Control-brain proteins show no binding. Bar=10 μm.

FIG. 33 presents data showing that fibril and protofibril binding is different from punctate ADDL binding. Rat hippocampal neurons were incubated with different Aβ1-42 preparations for 1 h, fixed, and immunolabeled using specific anti-ADDL rabbit antisera. Immunofluorescence images show differences in both structure and binding to cells. Fibrils (Left) appear as large structures not engaged in any specific cell “binding.” Seeded Aβ1-42 containing protofibrils (Center) form smaller, but heavily distributed structures attached to processes and cell bodies with no characteristic pattern. ADDLs (Right) appear as 0.2 m diameter binding hot spots. Microscopy shows ADDL binding is extremely non-uniform, consistent with selective binding to restricted cell surface domains. The ADDL-receptor “hot spots” are not random, and they occur most abundantly on neurites, which are regions of growth and plasticity.

FIG. 34 presents data showing that ADDL puncta show minimal incidence of co-localization with p75-NGF receptors. Dissociated rat hippocampal cells were cultured for 12 days prior to incubation with 1 uM ADDLs for 1.5 hrs at 37° C. Cells were fixed and double-labeled with a polyclonal anti-ADDLs antibody and a monoclonal anti-p75. Overlay of anti-ADDLs (red) and anti-p75 NTR (green) show minimal co-localization (yellow) of the two antibodies. Fluorescent overlay created with MetaMorph Imaging software. 100× magnification.

FIG. 35 presents data showing that ADDL receptor complexes are detected as puncta on processes labeled with anti-MAP-2a,b (dendrites). Dissociated rat hippocampal cells were cultured for 12 d prior to incubation with 1 μM ADDLs for 1.5 h at 37° C. Cells were fixed and double-labeled with a polyclonal anti-ADDLs antibody and a monoclonal anti-MAP2a,b. Overlay of anti-ADDLs (red) and anti-MAP-2a,b (green) show that ADDLs bind to MAP-2a,b, labeled processes. Fluorescent overlay created with MetaMorph Imaging software. 100× magnification.

FIG. 36 presents data showing that ADDLs bind to active membrane sites, showing localization to puncta even at lamellipodia tips. ADDL binding to NT2 growth cones can be detected via immunofluorescence using 6E10-B antibody. NT2 cells are incubated with ADDLs (5-10 μM) for 2 hours and then rinsed. Immunofluorescence results of NT2 cells at high magnifications reveal that ADDLs bind to discrete puncta at lamellipodia tips as well as the processes and cell body.

FIG. 37 presents data showing that ADDL receptor complexes localize to dendritic spines and post-synaptic sites.

FIG. 38 presents data showing that ADDL receptor puncta co-localize with vinculin. ADDL receptor localization with paxillin was negligible, except at a few focal contacts that contained paxillin (Top panels—lower row). Immunofluorescence assay detects ADDLs localizing to vinculin-positive puncta along the processes and cell bodies of cultured hippocampal cells. Certain of these puncta also indicate paxillin localization. Primary rat hippocampal cells are cultured for 4 days prior to incubation with 1 μM ADDLs for 1.5 hrs at 37° C. Cells are rinsed, fixed, and double-labeled with polyclonal anti-ADDL and monoclonal anti-vinculin or anti-paxillin antibodies. Fluorescent overlays (above) created with MetaMorph Imaging software and show that ADDLs (red) and vinculin (green—Top panels—upper row) show numerous sites of co-localization (yellow) along the processes and cell bodies. (Bottom panel) ADDL receptor punta (Green) show minimal overlap with paxillin (red).

FIG. 39 presents data showing that ADDL receptor binding increases the detectable levels of tyrosine-phosphorylated FAK. Western blotting reveals elevated FAK-YP within 1 h of ADDL treatment. The bright spots (top right) indicate the locations of FAK-YP. Quantitation (bottom left) reveals FAK-YP is elevated 3×. FAK-YP localizes with ADDL receptor complex puncta (lower right).

FIG. 40 presents data showing that toxic, low molecular weight oligomers are used as antigens to generate monoclonal antibodies. Panel A. Immunoblot (using polyclonal antibody M93/3) and silver stain of ADDLs used to immunize three mice (#1, #2, and #3, respectively); Panel B. Toxicity of these ADDLs at 5 μM as measure by a MTT assay in PC12 cells.

FIG. 41 presents data showing that mice mount a vigorous antigenic response to ADDLs. The Figure shows an immunoblot in which ˜20 pmol ADDLs are visualized with control rabbit oligomer-selective polyclonal antibody (M93/3) and with two dilutions (1:75, 1:100) of plasma from mouse #1.

FIG. 42 presents data showing that dual screening is effective for selecting hybridomas that target small molecular weight ADDLs. Left panel: Dot blot (5 pmol ADDLs) in which hybridoma supernates that bind ADDLs are selected, i.e. 3B7 and 3D8. Right panel: Immunoblots (20 pmol ADDLs) in which binding of hybridomas to selected molecular weight oligomers are screened. For example, #15 is 3B7 in top blot.

FIG. 43 presents data showing that hybridomas generated as described in Example 25 (see also FIGS. 40-42) target different molecular weight oligomers. The Figure shows an immunoblot in which various hybridoma supernates are used to visualize ADDLs (20 pmol/lane). Note that 3B7 recognizes only lower molecular weight ADDLs, while 5A9 and 11B5 recognize lower and higher molecular weight species. 8C3 may recognize only higher molecular weight oligomers. Expanded hybridomas were rescreened in the dot blot and immunoblot assays.

FIG. 44 presents data that demonstrates that anti-ADDL monoclonal antibody 3B7 identifies ADDL binding sites similar to polyclonal M94/3 on hippocampal cells. ADDLs (500 nM) are incubated for 6 hr with 21-day hippocampal cultures. The ADDLs are removed by washing and the cells are fixed with formaldehyde. The cells are then exposed to supernate from various hybridomas (1:5) or to rabbit polyclonal M94/3 (1:200) for 1.5 hr and then visualized with Alexa 488-conjugated anti-mouse secondary. Note the similarity of the puncta in the left images of 3B7 with that of M94/3 shown herein.

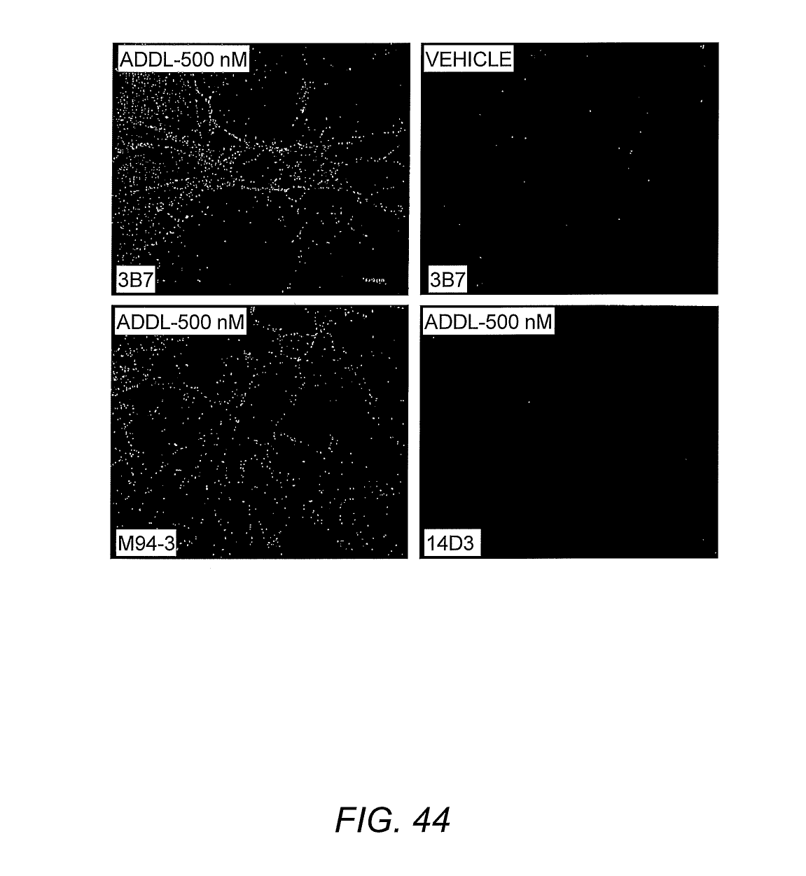

FIG. 45 presents data showing ADDLs separated by SDS-PAGE, blotted to nitrocellulose and incubated with 3B7 antibody, 11B5 antibody, or Control antibody (6E 10) according to standard procedures (Panels A and B). (A) Antibody 3B7 recognized various oligomer bands, but not monomer. Molecular weight markers are indicated on the left. (B) As above, except that estimated oligomer sizes are indicated on the left.

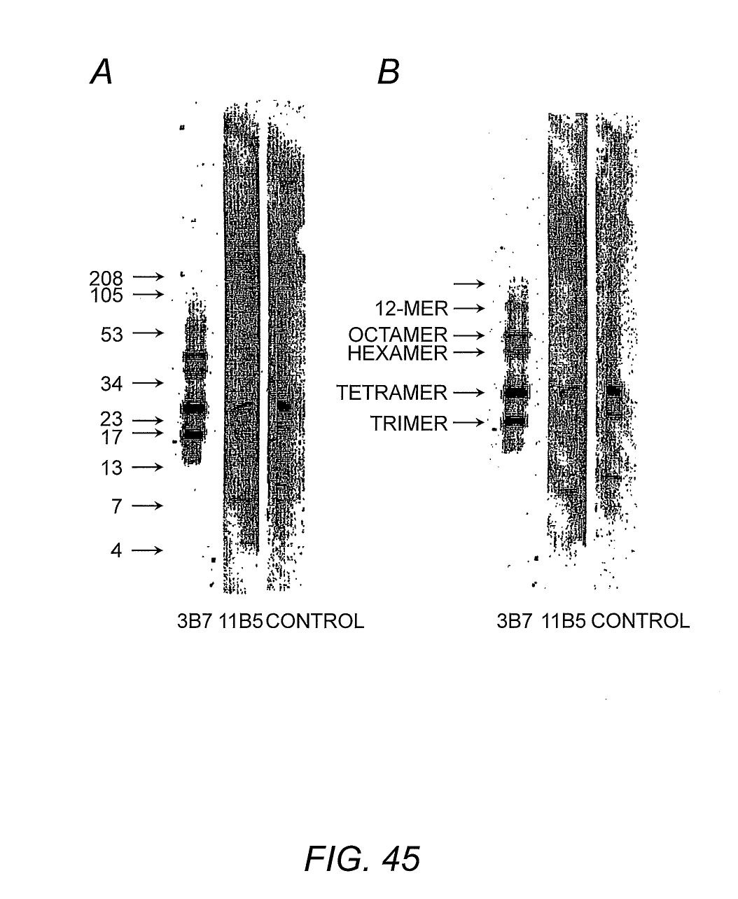

FIG. 46 presents data showing cortical sections of brain tissue from AD (top) or age-matched control (bottom) individuals were stained with ADDL-selective M93 antibody to visualize ADDLs. Cell nuclei were stained with a Hoechst stain. Standard protocols were followed for all procedures.

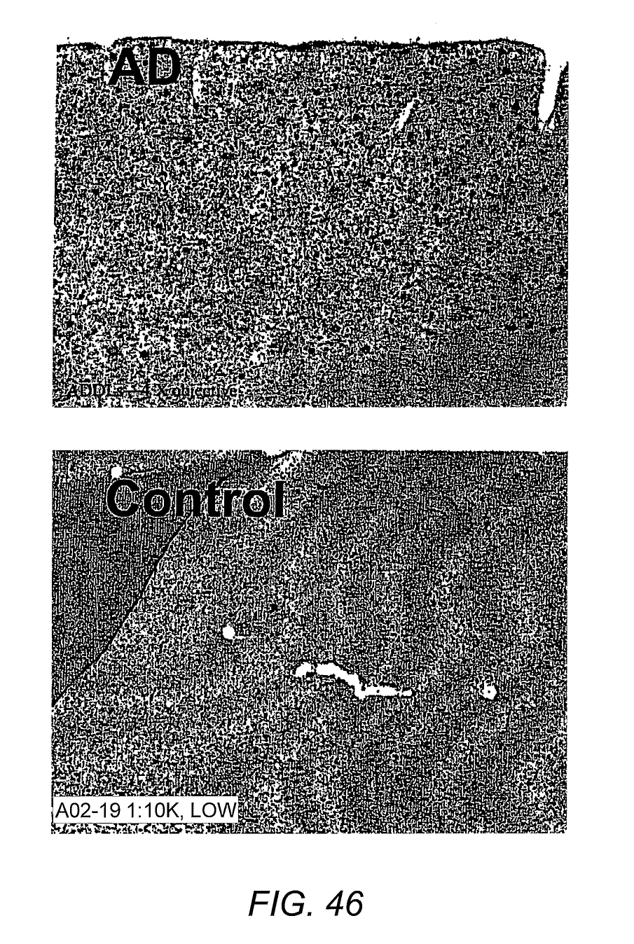

FIG. 47 presents data showing magnified regions from immunostained brain tissue from an AD individual reveal light gray staining around the surface of neurons, indication binding to neuron receptor proteins. The blue stain indicates the location of cell nuclei. Standard protocols were followed for all procedures.

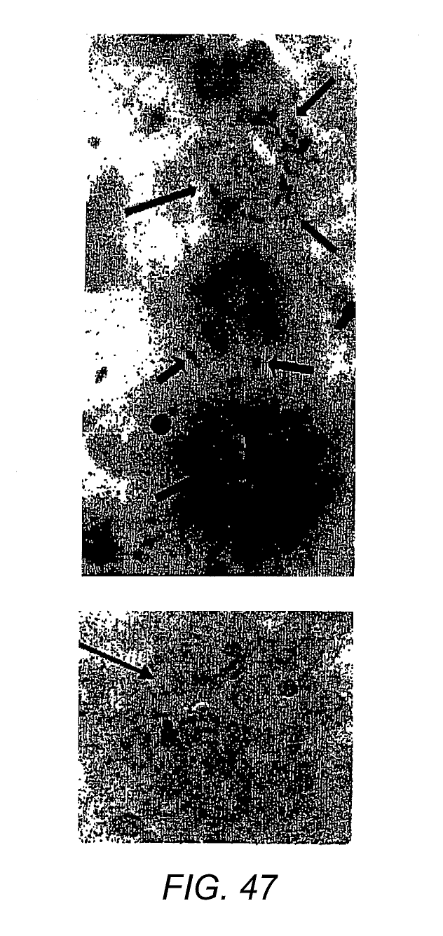

FIG. 48 presents data showing ADDL selective antibodies can be used for dot blot detection of ADDLs in blood or plasma, as well as brain tissue. The panel on the upper left shows that ADDLs from AD transgenic (Tg) mice are elevated in plasma (right-most bar), compared with non-Tg mice (second bar from left). The first and third bars in that panel show that ADDLs can be detected in Tg or normal mice after iv injection of ADDLs into the mice. Comparative data for ADDLs detected in brain tissue extracts are shown in the left panel. Standard curves for these diagnostic assays are shown in the lower panels. Standard protocols were followed for all procedures.

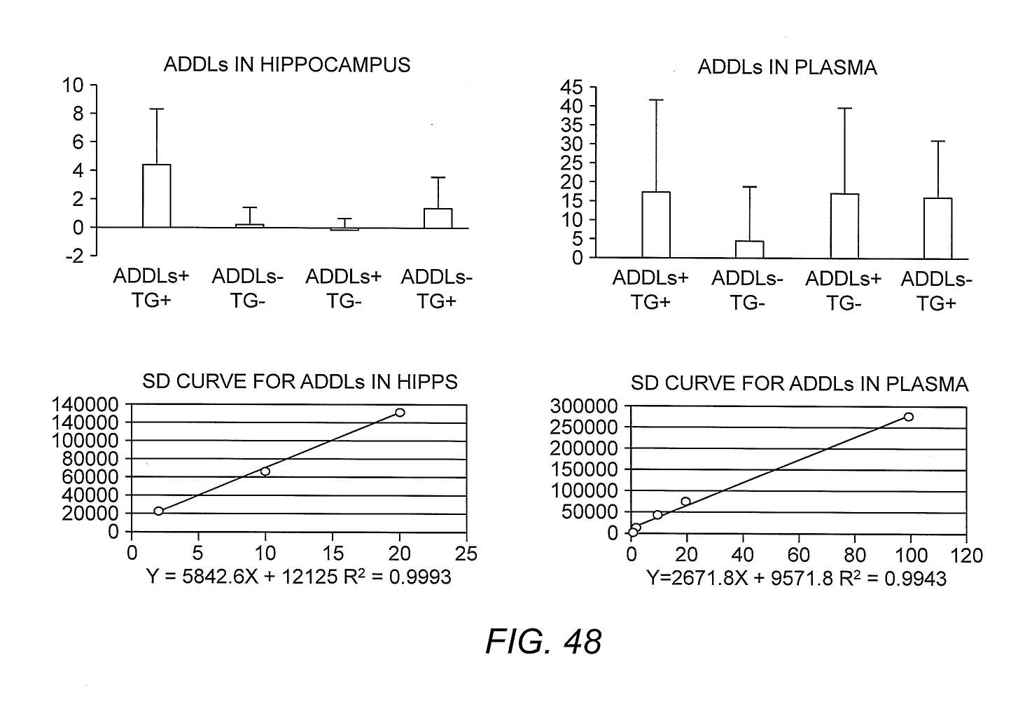

### DETAILED DESCRIPTION OF THE INVENTION

Aβ-derived oligomers (ADDLs) are effective antigens, eliciting antibodies that are analytically useful and potentially of therapeutic and prophylactic value. The antibodies discriminate oligomers from monomers, and they exhibit efficacy and specificity in immunoblots and immunofluorescence microscopy. The antibodies, moreover, neutralize the biological activity of ADDLs. This is significant because emerging evidence suggests that ADDLs are the relevant pathogenic molecules that form when levels of Aβ₁₋₄₂ become elevated. Unlike deposited amyloid, ADDLs are small neurotoxins that are soluble and diffusible. They have been demonstrated to interfere directly with the key electrophysiology and biochemistry required for information storage, namely LTP. Therefore, the ability to neutralize these soluble toxins may be highly significant for therapeutic intervention in Alzheimer's disease and related disorders.

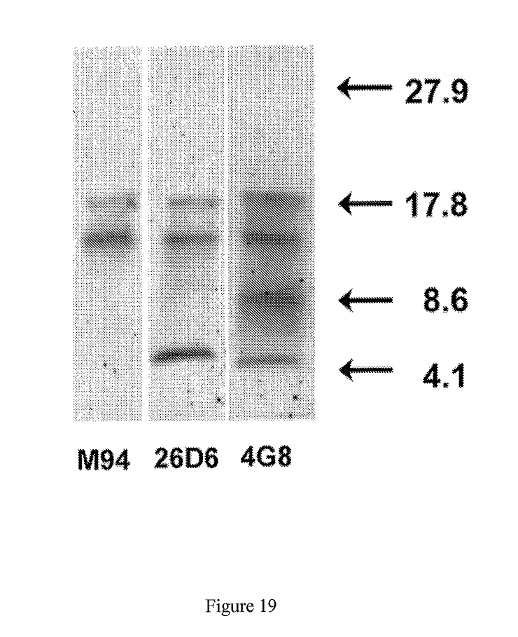

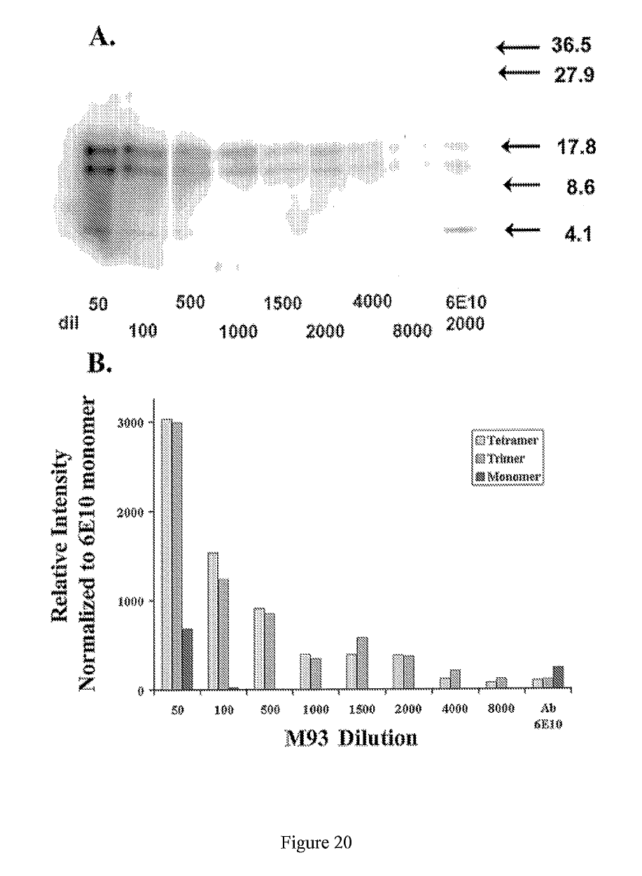

The antibodies induced by ADDL preparations show specificity for oligomers. In some instances, monomers can be detected at very high doses of antibodies, but serial dilutions establish that antibodies from several animals (designated 90, 93 or 94) preferentially recognize and bind to oligomers (FIG. 19 and FIG. 20). It should be noted these ADDL preparations do not convert to protofibrils or fibrils, eliminating the possibility that these larger assemblies could be responsible for generating the observed immune response.

Several possibilities could cause oligomers to be more antigenic than monomer. One possibility might be that the oligomers may be inherently more immunogenic due to presentation of novel, conformationally dependent epitopes, absent from monomer. Monomers also are likely to be intrinsically less immunogenic because of their physiological role consequent to normal metabolism of APP molecules (Selkoe, D. J. (1994) Annu. Rev. of Neurosci., vol. 17, pp. 489-517), which are transiently abundant during development (Enam, S. A. (1991) Ph.D. Thesis, Northwestern University). Another possibility might be that monomers may be cleared more efficiently than oligomers.

The binding affinities and detection efficacies of ADDL-antibodies are comparable to commercial Aβ monoclonal antibodies (FIG. 19). For example, at higher ADDL concentrations (100 pmol), ADDL-antibodies at 0.3 μg/ml show a binding intensity comparable to that of commercial monoclonal antibodies used at 0.4 to 0.5 μg/ml (FIG. 19). These commercial monoclonals also recognized epitopes common to several states of Aβ assembly, including monomers and dimers, which were not detected by anti-ADDL antibodies. That alternative assembly-states of Aβ manifest different epitopes is in harmony with their differing toxic activities, a property that may be exploited for future drug development. ADDL-antibodies also show efficacies that are as least as good as monoclonal antibodies when used at very low Aβ concentrations (Ida, N. et al. (1996) J. Biol. Chem., vol. 271, pp. 22908-22914; Potempska, A. et al. (1999) Amyloid, vol. 6, pp. 14-21). Immunoblots with ADDL-antibodies at a final IgG protein concentration of 0.6 μg/ml can recognize less than 1 fmol of ADDLs.

Besides potency, the antibodies show significant specificity, making them useful for analytical experiments. This is not always the case for other antibodies produced against Aβ peptides. For example, some monoclonal antibodies against Aβ₃₅₋₄₂ and Aβ₃₃₋₄₀ bind non-specifically to components in CSF and blood plasma on immunoblots, even though they are selective for Aβ in an ELISA (Ida, N. et al. (1996) J. Biol. Chem., vol. 271, pp. 22908-22914). The M93 and M94 antibodies (see below) showed no binding to proteins in total rat homogenate, in harmony with their selectivity for oligomer over monomer. Similarly, in immunofluorescence microscopy experiments, the antibodies showed little binding to cell surfaces in the absence of exogenous ADDLs.

Two interesting observations emerge from the immunoblot and immunofluorescence experiments. First, when ADDLs were mixed with brain homogenates, immunoblots showed ADDLs at their normal molecular weight range, but, in addition, species at a higher molecular weight were also observed. The basis for this addition is not known, but it previously has been established that several different proteins can influence the aggregation properties of Aβ (Klein, W. L. (2000) in Molecular Mechanisms of Neurodegenerative Diseases (Chesselet, M.-F., Ed.), Humana Press; Klein, W. L. et al. (2001) Trends Neurosci., vol. 24, pp. 219-224). The size of the species seen here (˜30-40 kDa) is the same as the size suggested to be a predominant form in AD-afflicted brain (Guerette, P. A. et al. (2000) Soc. Neurosci. Abstr., vol. 25, p. 2129). However, the additional species may also be tightly-adherent ADDLs bound to a small brain protein, e.g., ApoE. A stable complex between Aβ and ApoE has been seen previously (LaDu, M. J. et al. (1997) J. Neurosci. Res., vol. 49, pp. 9-18; LaDu, M. J. et al. (1995) J. Biol. Chem., vol. 270, pp. 9039-9042). Second, from neuron culture experiments, immunofluorescence data showed ADDLs became associated with neurons in a highly patterned manner. The nature of these “hot spots” suggests possible receptor involvement in ADDL toxicity (Viola, Gong, Lambert, Lin, and Klein, in preparation).

Somewhat surprising and potentially most significant is the neuroprotection afforded by antibodies at substoichiometric doses. Tests of protection used the MTT reduction assay with PC12 neuron-like cells. In this bioassay, which monitors exocytotsis/endocytosis as well as oxidative metabolism (Liu, Y. & Schubert, D. (1997) J. Neurochem., vol. 69, pp. 2285-2293), ADDLs maximally block MTT reduction at doses of 1-5 μM.

Substoichiometric levels of antibodies blocked the ADDL impact, with blockade evident at antibodies/ADDL molar ratios as low as to 1:15. This efficacy is similar to data reporting that guinea pig antibodies can prevent toxicity of amyloid in a PC12 MTT assay at a ratio of 1:20 (Frenkel, D. et al. (2000) Proc. Natl. Acad. Sci. USA, vol. 97, pp. 11455-11459). In the present case, low relative doses of antibodies appear protective because of their selectivity for toxic oligomers (FIGS. 19 and 20). Monomer is not toxic (Yanker, B. A. (1996) Neuron, vol. 16, pp. 921-932; Yanker, B. A. et al. (1989) Science, vol. 245, pp. 417-420), but makes up 45+/−5% of the total soluble Aβ (Chromy, B. C. et al., in preparation). The antibodies thus appear to target and lower the availability of toxic subspecies in the ADDL solution.

Antibodies that target toxic forms of self-assembled Aβ have become of great interest because of the remarkable recent findings that antibodies against Aβ cross the blood brain barrier and are therapeutic in transgenic mice models of AD (Bard, F. et al. (2000) Nature Med., vol. 6, pp. 916-919; Schenk, D. (1999) Nature, vol. 400, pp. 173-177). The vaccination protocols lead to loss of amyloid (Bard, F. et al. (2000) Nature Med., vol. 6, pp. 916-919; Schenk, D. (1999) Nature, vol. 400, pp. 173-177) and are effective in preventing behavior decline (Helmuth, L. (2000) Science, vol. 289, p. 375; Arendash, G. et al. (2000) Soc. Neurosci. Abstr., vol. 26, p. 1059; Yu. W. et al. (2000) Soc. Neurosci. Abstr., vol. 26, p. 497). The authors of these immunization/vaccination studies have suggested that therapeutic efficacy may be due indirectly to activated microglia, which remove amyloid plaque proteins. Other studies, however, have shown that antibodies made in bacteria and mammals by phage display can directly bring about dissociation of aggregated Aβ in vitro (Frenkel, D. et al. (2000) Proc. Natl. Acad. Sci. USA, vol. 97, 11455-11459; Frenkel, D. et al. (2000) J. Neuroimnunmol., vol. 106, pp. 23-31). These antibodies are produced against the EFRH epitope, amino acids #3-6 of Aβ. This site is hypothesized to be the regulatory site on N-terminals of fibrils (Frenkel, D. et al. (1998) J. Neuroinnmunol., vol. 88, pp. 85-90).

An alternative explanation for the behavioral efficacy of these antibodies is that they may neutralize soluble ADDLs, which putatively play a pathogenic role in transgenic mice AD models and in AD itself. Multiple transgenic APP mice models show behavioral and degenerative losses in the complete absence of amyloid deposits (Klein, W. L. (2000) in Molecular Mechanisms of Neurodegenerative Diseases (Chesselet, M.-F., Ed.), Humana Press; Klein, W. L. et al. (2001) Trends Neurosci., vol. 24, pp. 219-224). Recently, e.g., amyloid-free APP-transgenic mice were found to exhibit loss of synaptophysin-immunoreactive terminals, a good measure of cognitive decline in AD (Terry, R. D. (1999) in Alzheimer's Disease (Terry, R. D. et al., Eds.), pp. 187-206, Lippincott Williams & Wilkins), in a manner that correlates nonetheless with levels of soluble Aβ₁₋₄₂ species (Mucke, L. et al. (2000) J. Neurosci., vol. 20, pp. 4050-4058). The authors suggest their results support an emerging view that plaque-independent Aβ toxicity is important in the development of synaptic deficits in AD. Analogous correlation between synapse loss and soluble AD has been observed in AD (Lue, L. F. et al. (1999) Am. J. Pathol., vol. 155, pp. 853-862; (Klein, W. L. (2000) in Molecular Mechanisms of Neurodegenerative Diseases (Chesselet, M.-F., Ed.), Humana Press; Klein, W. L. et al. (2001) Trends Neurosci., vol. 24, pp. 219-224; McLean, C. A. et al. (1999) Ann. Neurol., vol. 46, pp. 860-866). Soluble toxic oligomers likely are key factors in plaque-independent Aβ toxicity. These findings, coupled with antibody data presented here, strongly suggest that behavioral improvement could, at least in part, also be a plaque-independent phenomenon.

Antibodies that target ADDLs may give the ideal specificity. The current neutralizing antibodies, which target novel domains dependent on peptide assembly, are proposed as prototypes for therapeutic vaccination. It is predicted that use of homologous antibodies would combat memory deficits in early stages of AD. By binding to ADDLs, antibodies would protect neural plasticity, which is inhibited experimentally at low ADDL doses (Lambert, M. P. et al. (1998) Proc. Natl. Acad. Sci. USA, vol. 95, pp. 6448-6453; Wang, H. et al. (2000) Soc. Neurosci. Abstr., vol. 26, pp. 1787). In addition, by targeting sub-fibrillar species, the antibodies would eliminate intermediates needed for plaque formation. Independent of their potential direct therapeutic value, the antibodies should be powerful tools to identify toxic domains on oligomer surfaces, thus providing critical molecular insight for development of more traditional therapeutic drugs. Moreover, ADDL-selective antibodies provide a basis for simple high throughput assays to screen libraries for compounds that block toxic oligomerization.

It has been discovered that in neurotoxic samples of amyloid 3 not only do fibrillar structures exist, but also, unexpectedly, some globular protein structures exist that appear to be responsible for the neurotoxicity. Using novel methods, samples that contain predominantly these soluble globular protein assemblies and no fibrillar structures have been generated as described herein. In heterogeneous samples prepared by various methods, the removal of the larger, fibrillar forms of amyloid β by centrifugation does not remove these soluble globular assemblies of amyloid β in the supernatant fractions. These supernatant fractions exhibit significantly higher neurotoxicity than non-fractionated amyloid P samples aggregated under literature conditions. These novel and unexpected neurotoxic soluble globular forms are referred to herein as amyloid β-derived dementing ligands, amyloid β-derived diffusible ligands (ADDLs), amyloid β soluble non-fibrillar structures, amyloid β oligomeric structures, or simply oligomeric structures. Samples of amyloid β that had been “aged” under standard literature conditions (e.g., Pike et al. (1993) J. Neurosci., vol. 13, pp. 1676-1687) for more than three weeks lose their neurotoxicity, even though these samples contain predominantly fibrillar structures with few or no ADDLs. This discovery that the globular ADDLs are neurotoxic is particularly surprising since current thinking holds that it is fibril structures that constitute the toxic form of amyloid β (Lorenzo et al. (1994) Proc. Natl. Acad. Sci. USA, vol. 91, pp. 12243-12247; Howlett et al. (1995) Neurodegen., vol. 4, pp. 23-32).

ADDLs can be formed in vitro. When a solution (e.g., a DMSO solution) containing monomeric amyloid β1-42 (or other appropriate amyloid β, as further described herein) is diluted into cold tissue culture media (e.g., F12 cell culture media), then allowed to incubate at about 4° C. for from about 2 to about 48 hours and centrifuged for about 10 minutes at about 14,000 g at a temperature of 4° C., the supernatant fraction contains small, soluble oligomeric globules that are highly neurotoxic, e.g., in neuronal cell and brain slice cultures. The ADDLs also can be formed by co-incubation of amyloid P with certain appropriate agents, e.g., clusterin (a senile plaque protein that also is known as ApoJ), as well as by other methods, as described herein.

Thus, in particular, the present invention pertains to an isolated, soluble, non-fibrillar amyloid β oligomeric structure. The oligomeric structure so isolated does not contain an exogenously added crosslinking agent. The oligomeric structure desirably is stable in the absence of any crosslinker.

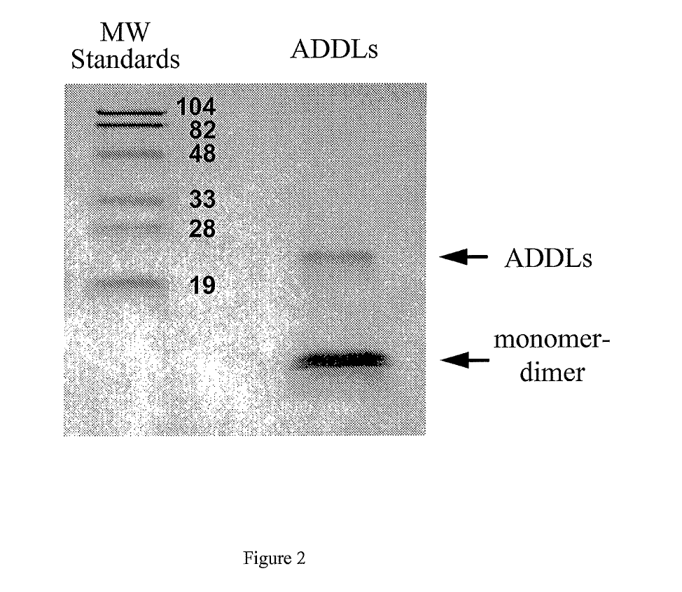

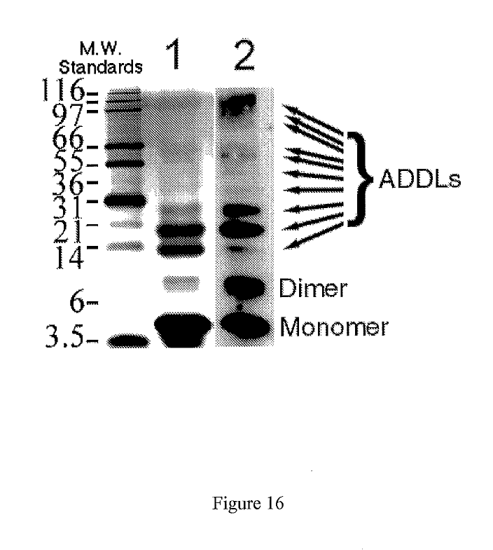

Atomic force microscope analysis (AFM) can be carried out as is known in the art and described herein, for instance, using a Digital Instruments Atomic force microscope as described in Example 3. AFM of such a supernatant fraction (i.e., a supernatant fraction in which fibrillar structures have been removed) reveals a number of different size globules (i.e., or different size oligomeric structures) present in the fraction. These globules fall within the range of from about 4.7 to about 11.0 nm, with the major fraction falling within a size range of from about 4.7 nm to about 6.2 nm. There appear to be distinct species of globules falling within this size range and which correspond to specific size oligomeric species such as those indicated by analysis on certain gel electrophoresis systems, as shown in FIG. 2 and FIG. 16. Slight variation in height surface results from how the particular species are seated on the mica surface at the time of AFM analysis. Despite this slight variation however, there appear to be several predominant sizes of globules in the 4.7-6.2 size range, i.e., from about 4.9 nm to about 5.4 nm, and from about 5.7 nm to about 6.2 nm, that constitute about 50% of the oligomeric structures in a typical sample. There also appears to be a distinct size species of globule having dimensions of from about 5.3 nm to about 5.7 nm. Larger globules from about 6.5 nm to about 11.0 nm appear less frequently, but may possess neurotoxic properties similar to the more prevalent, smaller species. It appears that the globules of dimensions of from about 4.7 nm to about 6.2 nm on AFM comprise the pentamer and hexamer form of oligomeric amyloid β (Aβ) protein. The AFM size globules of from about 4.2 nm to about 4.7 nm appear to correspond to the Aβ tetramer. The size globules of from about 3.4 nm to about 4.0 nm to appear to correspond to trimer. The large globules appear to correspond to oligomeric species ranging in size from about 13 amyloid monomers to about 24 amyloid monomers. The size globules of from about 2.8 nm to about 3.4 nm correspond to dimer (Roher et al. (1996) J. Biol. Chem., vol. 271, pp. 20631-20635). The Aβ monomer AFM size ranges from about 0.8 nm to about 1.8-2.0 nm. Monomeric and dimeric amyloid β are not neurotoxic in neuronal cell cultures or in the organotypic brain slice cultures.

Thus, the present invention provides an isolated soluble non-fibrillar amyloid β oligomeric structure (i.e., an ADDL) that preferably comprises at from about 3 to about 24 amyloid β protein monomers, especially from about 3 to about 20 amyloid β protein monomers, particularly from about 3 to about 16 amyloid β protein monomers, most preferably from about 3 to about 12 amyloid β protein monomers, and which desirably comprises at from about 3 to about 6 amyloid β protein monomers. As previously described, large globules (less predominant species) appear to correspond to oligomeric species ranging in size from about 13 amyloid β monomers to about 24 amyloid β monomers. Accordingly, the invention provides an isolated soluble non-fibrillar amyloid β oligomeric structure wherein the oligomeric structure preferably comprises trimer, tetramer, pentamer, hexamer, heptamer, octamer, 12-mer, 16-mer, 20-mer or 24-mer aggregates of amyloid β proteins. In particular, the invention provides an isolated soluble non-fibrillar amyloid β protein oligomeric structure wherein the oligomeric structure preferably comprises trimer, tetramer, pentamer, or hexamer aggregates of amyloid β protein. The oligomeric structure of the invention optimally exhibits neurotoxic activity.

The higher order structure of the soluble, non-fibrillar amyloid β protein oligomer structure (i.e., the aggregation of monomers to form the oligomeric structure) desirably can be obtained not only from amyloid β1-42, but also from any amyloid β protein capable of stably forming the soluble non-fibrillar amyloid β oligomeric structure. In particular, amyloid β1-43 also can be employed. Amyloid β1-42 with biocytin at position 1 also can be employed. Amyloid β (e.g., β1-42 or β1-43) with a cysteine at the N-terminus also can be employed. Similarly, Aβ truncated at the amino terminus (e.g., particularly missing one or more up to the entirety of the sequence of amino acid residues 1 through 8 of Aβ1-42 or Aβ1-43), or Aβ (e.g., Aβ1-42 or 1-43) having one or two extra amino acid residues at the carboxyl terminus can be employed. By contrast, amyloid β1-40 can transiently form ADDL-like structures which can be toxic, but these structures are not stable and cannot be isolated as aqueous solutions, likely due to the shortened nature of the protein, which limits its ability to form such higher order assemblies in a stable fashion.

Desirably, the isolated soluble non-fibrillar amyloid β oligomeric structure according to the invention comprises globules of dimensions of from about 4.7 nm to about 11.0 nm, particularly from about 4.7 nm to about 6.2 nm as measured by atomic force microscopy. Also, preferably the isolated soluble non-fibrillar amyloid β oligomeric structure comprises globules of dimensions of from about 4.9 nm to about 5.4 nm, or from about 5.7 nm to about 6.2 nm, or from about 6.5 nm to about 11.0 nm, as measured by atomic force microscopy. In particular, preferably the isolated soluble non-fibrillar amyloid β oligomeric structure according to the invention is such that wherein from about 30% to about 85%, even more preferably from about 40% to about 75% of the assembly comprises two predominant sizes of globules, namely, of dimensions of from about 4.9 nm to about 5.4 nm, and from about 5.7 nm to about 6.2 nm, as measured by atomic force microscopy. However, it also is desirable that the oligomeric structure comprises AFM size globules of about 5.3 to about 5.7 nm. It is also desirable that the oligomeric structure may comprise AFM size globules of about 6.5 nm to about 11.0 nm.

By non-denaturing gel electrophoresis, the bands corresponding to ADDLs run at about from 26 kD to about 28 kD, and with a separate broad band representing sizes of from about 36 kD to about 108 kD. Under denaturing conditions (e.g., on a 15% SDS-polyacrylamide gel), the ADDLs comprise a band that runs at from about 22 kD to about 24 kD, and may further comprise a band that runs at about 18 to about 19 kD. Accordingly, the invention preferably provides an isolated soluble non-fibrillar amyloid β oligomeric structure (i.e., ADDL) that has a molecular weight of from about 26 kD to about 28 kD as determined by non-denaturing gel electrophoresis. The invention also preferably provides an isolated soluble non-fibrillar amyloid β oligomeric structure (i.e., ADDL) that runs as a band corresponding to a molecular weight of from about 22 kD to about 24 kD as determined by electrophoresis on a 15% SDS-polyacrylamide gel. The invention further preferably provides an isolated soluble non-fibrillar amyloid β oligomeric structure (i.e., ADDL) that runs as a band corresponding to a molecular weight of from about 18 kD to about 19 kD as determined by electrophoresis on a 15% SDS-polyacrylamide gel.

Also, using a 16.5% tris-tricine SDS-polyacrylamide gel system, additional ADDL bands can be visualized. The increased resolution obtained with this gel system confirms the ability to obtain according to the invention an isolated oligomeric structure having a molecular weight ranging from about 13 kD to about 116 kD, as determined by electrophoresis on a 16.5% tris-tricine SDS-polyacrylamide gel. The ADDL bands appear to correspond to distinct size species. In particular, use of this gel system allows visualization of bands corresponding to trimer with a size of about 13 to about 14 kD, tetramer with a size of about 17 to about 19 kD, pentamer with a size of about 22 kD to about 23 kD, hexamer with a size of about 26 to about 28 kD, heptamer with a size from about 32 kD to 33 kD, and octamer with a size from about 36 kD to about 38 kD, as well as larger soluble oligomers ranging in size from about 12 monomers to about 24 monomers. Thus, the invention desirably provides an isolated oligomeric structure, wherein the oligomeric structure has, as determined by electrophoresis on a 16.5% tris-tricine SDS-polyacrylamide gel, a molecular weight selected from the group consisting of from about 13 kD to about 14 kD, from about 17 kD to about 19 kD, from about 22 kD to about 23 kD, from about 26 kD to about 28 kD, from about 32 kD to about 33 kD, and from about 36 kD to about 38 kD.

The invention further provides a method for preparing the isolated, soluble, non-fibrillar amyloid β oligomeric structure. This method optionally comprises the steps of:
(a) obtaining a solution of monomeric amyloid β protein;
(b) diluting the protein solution into an appropriate media;
(c) incubating the media resulting from step (b) at about 4° C.;
(d) centrifuging the media at about 14,000 g at about 4° C.; and
(e) recovering the supernatant resulting from the centrifugation as containing the amyloid β oligomeric structure.

In step (c) of this method, the solution desirably is incubated for about 2 hours to about 48 hours, especially for about 12 hours to about 48 hours, and most preferably for about 24 hours to about 48 hours. In step (d) of this method, the centrifugation preferably is carried out for about 5 minutes to about 1 hour, especially for about 5 minutes to about 30 minutes, and optimally for about 10 minutes. Generally, however, this is just a precautionary measure to remove any nascent fibrillar or protofibrillar structures and may not be necessary, particularly where long-term stability of the ADDL preparation is not an issue.

The Aβ protein is diluted in step (b) desirably to a final concentration ranging from about 5 nM to about 500 μM, particularly from about 5 μM to about 300 μM, especially at about 100 μM. The “appropriate media” into which the Aβ protein solution is diluted in step (b) preferably is any media that will support, if not facilitate, ADDL formation. In particular, F12 media (which is commercially available as well as easily formulated in the laboratory) is preferred for use in this method of the invention. Similarly, “substitute F12 media” also desirably can be employed. Substitute F12 media differs from F12 media that is commercially available or which is formulated in the laboratory. According to the invention, substitute F12 media preferably comprises the following components: N,N-dimethylglycine, D-glucose, calcium chloride, copper sulfate pentahydrate, iron (II) sulfate heptahydrate, potassium chloride, magnesium chloride, sodium chloride, sodium bicarbonate, disodium hydrogen phosphate, and zinc sulfate heptahydrate.

In particular, synthetic F12 media according to the invention optionally comprises: N, N-dimethylglycine (from about 600 to about 850 mg/L), D-glucose (from about 1.0 to about 3.0 g/L), calcium chloride (from about 20 to about 40 mg/L), copper sulfate pentahydrate (from about 15 to about 40 mg/L), iron (II) sulfate heptahydrate (from about 0.4 to about 1.2 mg/L), potassium chloride (from about 160 to about 280 mg/L), magnesium chloride (from about 40 to about 75 mg/L), sodium chloride (from about 6.0 to about 9.0 g/L), sodium bicarbonate (from about 0.75 to about 1.4 g/L), disodium hydrogen phosphate (from about 120 to about 160 mg/L), and zinc sulfate heptahydrate (from about 0.7 to about 1.1 mg/L). Optimally, synthetic F12 media according to the invention comprises: N,N-dimethylglycine (about 766 mg/L), D-glucose (about 1.802 g/L), calcium chloride (about 33 mg/L), copper sulfate pentahydrate (about 25 mg/L), iron (II) sulfate heptahydrate (about 0.8 mg/L), potassium chloride (about 223 mg/L), magnesium chloride (about 57 mg/L), sodium chloride (about 7.6 g/L), sodium bicarbonate (about 1.18 g/L), disodium hydrogen phosphate (about 142 mg/L), and zinc sulfate heptahydrate (about 0.9 mg/L). Further, the pH of the substitute F12 media preferably is adjusted, for instance, using 0.1 M sodium hydroxide, desirably to a pH of about 7.0 to about 8.5, and preferably a pH of about 8.0.

The foregoing method further desirably can be carried out by forming the slowly-sedimenting oligomeric structure in the presence of an appropriate agent, such as clusterin. This is done, for instance, by adding clusterin in step (c), and, as set out in the Examples which follow.

Moreover, the invention also provides as described in the Examples, a method for preparing a soluble non-fibrillar amyloid β oligomeric structure according to the invention, wherein the method comprises:
(a) obtaining a solution of monomeric amyloid β protein, the amyloid β protein being capable of forming the oligomeric structure;
(b) dissolving the amyloid β monomer in hexafluoroisoproanol;
(c) removing hexafluoroisoproanol by speed vacuum evaporation to obtain solid peptide;
(d) dissolving the solid peptide in DMSO to form a DMSO stock solution;
(e) diluting the stock solution into an appropriate media;
(f) vortexing; and
(g) incubating at about 4° C. for about 24 hours.

If the ADDLs are prepared by the incorporation of 10% biotinylated amyloid β1-42 (or other appropriate biotinylated amyloid β protein), they can be utilized in a receptor binding assay using neural cells and carried out, for instance, on a fluorescence activated cell sorting (FACS) instrument, with labeling by a fluorescent avidin conjugate. Alternately, instead of incorporating biotin in the amyloid β protein, another reagent capable of binding the ADDL to form a fluorescently labeled molecule, and which may already be part of a fluorescent-labeled conjugate, can be employed. For instance, the soluble non-fibrillar amyloid β oligomeric structure can be formed such that the amyloid protein includes another binding moiety, with “binding moiety” as used herein encompassing a molecule (such as avidin, streptavidin, polylysine, and the like) that can be employed for binding to a reagent to form a fluorescently-labeled compound or conjugate. The “fluorescent reagent” to which the oligomeric structure binds need not itself fluoresce directly, but instead may merely be capable of fluorescence through binding to another agent. For example, the fluorescent reagent that binds the oligomeric structure can comprise a P amyloid specific antibody (e.g., 6E10), with fluorescence generated by use of a fluorescent secondary antibody.

Along with other experiments, FACSscan analysis of the rat CNS B103 cells was done without and with ADDL incubation. Results of these and further studies confirm that binding to the cell surface is saturable, and brief treatment with trypsin selectively removes a subset of cell surface proteins and eliminates binding of ADDLs. Proteins that are cleavable by brief treatment with trypsin from the surface of B 103 cells also prevent ADDL binding to B103 cells or cultured primary rat hippocampal neurons. These results all support that the ADDLs act through a particular cell surface receptor, and that early events mediated by the ADDLs (i.e., events prior to cell killing) can be advantageously controlled (e.g., for treatment or research) by compounds that block formation and activity (e.g., including receptor binding) of the ADDLs.

Thus, the invention provides a method for identifying compounds that modulate (i.e., either facilitate or block) activity (e.g., activity such as receptor binding) of the ADDL. This method preferably comprises:
(a) contacting separate cultures of neuronal cells with the oligomeric structure of the invention either in the presence or absence of contacting with the test compound;
(b) adding a reagent that binds to the oligomeric structure, the reagent being fluorescent;
(c) analyzing the separate cell cultures by fluorescence-activated cell sorting; and
(d) comparing the fluorescence of the cultures, with compounds that block activity (i.e., binding to a cell surface protein) of the oligomeric structure being identified as resulting in a reduced fluorescence of the culture, and compounds that facilitate binding to a cell surface protein (i.e., a receptor) being identified as resulting in an increased fluorescence of the culture, as compared to the corresponding culture contacted with the oligomeric structure in the absence of the test compound.

Alternately, instead of adding a fluorescent reagent that in and of itself is able to bind the protein complex, the method desirably is carried out wherein the oligomeric structure is formed from amyloid β1-42 protein (or another amyloid β) prepared such that it comprises a binding moiety capable of binding the fluorescent reagent.

Similarly, the method can be employed for identifying compounds that modulate (i.e., either facilitate or block) formation or activity (e.g., binding to a cell surface protein, such as a receptor) of the oligomeric structure comprising:
(a) preparing separate samples of amyloid β that either have or have not been mixed with the test compound;
(b) forming the oligomeric structure in the separate samples;
(c) contacting separate cultures of neuronal cells with the separate samples;
(d) adding a reagent that binds to the oligomeric structure, the reagent being fluorescent;
(e) analyzing the separate cell cultures by fluorescence-activated cell sorting; and
(f) comparing the fluorescence of the cultures, with compounds that block formation or binding to a cell surface protein of the oligomeric structure being identified as resulting in a reduced fluorescence of the culture, and compounds that facilitate formation or binding to a cell surface protein of the oligomeric structure being identified as resulting in an increased fluorescence of the culture, as compared to the corresponding culture contacted with the oligomeric structure in the absence of the test compound.

Further, instead of adding a fluorescent reagent that in and of itself is able to bind the protein complex, the method can be carried out wherein the oligomeric structure is formed from amyloid β protein prepared such that it comprises a binding moiety capable of binding the fluorescent reagent.

The fluorescence of the cultures further optionally is compared with the fluorescence of cultures that have been treated in the same fashion except that instead of adding or not adding test compound prior to formation of the oligomeric structure, the test compound either is or is not added after formation of the oligomeric structure. In this situation, compounds that block formation of the oligomeric structure are identified as resulting in a reduced fluorescence of the culture, and compounds that facilitate formation of the oligomeric structure are identified as resulting in an increased fluorescence of the culture, as compared to the corresponding culture contacted with the oligomeric structure in the absence of the test compound, only when the compound is added prior to oligomeric structure.

By contrast, compounds that block binding to a cell surface protein (e.g., a receptor) of the oligomeric structure are identified as resulting in a reduced fluorescence of the culture, and compounds that facilitate binding to a cell surface protein of the oligomeric structure are identified as resulting in an increased fluorescence of the culture, as compared to the corresponding culture contacted with the oligomeric structure in the absence of the test compound, when the compound is added either prior to or after oligomeric structure.

In a similar fashion, a cell-based assay, particularly a cell-based enzyme-linked immunosorbent assay (ELISA) can be employed in accordance with the invention to assess ADDL binding activity. In particular, the method can be employed to detect binding of the oligomeric structure to a cell surface protein. This method preferably comprises:
(a) forming an oligomeric structure from amyloid β protein;
(b) contacting a culture of neuronal cells with the oligomeric structure;
(c) adding an antibody (e.g., 6E10) that binds said oligomeric structure, said antibody including a conjugating moiety (e.g., biotin, or other appropriate agent);
(d) washing away unbound antibody;
(e) linking an enzyme (e.g., horseradish peroxidase) to said antibody bound to said oligomeric structure by means of said conjugating moiety;
(f) adding a colorless substrate (e.g., ABTS) that is cleaved by said enzyme to yield a color change; and
(g) determining said color change (e.g., spectrophotometrically) or the rate of the color change as a measure of binding to a cell surface protein (e.g., a receptor) of said oligomeric structure.

As earlier described, the antibody can be any antibody capable of detecting ADDLs (e.g., an antibody specific for ADDLs or an antibody directed to an exposed site on amyloid β), and the antibody conjugating moiety can be any agent capable of linking a means of detection (e.g., an enzyme). The enzyme can be any moiety (e.g., perhaps even other than a protein) that provides a means of detecting (e.g., color change due to cleavage of a substrate), and further, can be bound (e.g., covalent or noncovalent) to the antibody bound to the oligomeric structure by means of another moeity (e.g., a secondary antibody). Also, preferably according to the invention the cells are adhered to a solid substrate (e.g., tissue culture plastic) prior to the conduct of the assay. It goes without saying that desirably step (b) should be carried out as described herein such that ADDLs are able to bind to cells. Similarly, preferably step (c) should be carried out for a sufficient length of time (e.g., from about 10 minutes to about 2 hours, desirably for about 30 minutes) and under appropriate conditions (e.g., at about room temperature, preferably with gentle agitation) to allow antibody to bind to ADDLs. Further, appropriate blocking steps can be carried out such as are known to those skilled in the art using appropriate blocking reagents to reduce any nonspecific binding of the antibody. The artisan is familiar with ELISAs and can employ modifications to the assay such as are known in the art.

The assay desirably also can be carried out so as to identify compounds that modulate (i.e., either facilitate or block) formation or binding to a cell surface protein of the oligomeric structure. In this method, as in the prior-described assays for test compounds, the test compound is either added to the ADDL preparation, prior to the contacting of the cells with the ADDLs. This assay thus can be employed to detect compounds that modulate formation of the oligomeric structure (e.g., as previously described). Moreover, the test compound can be added to the ADDL preparation prior to contacting the cells (but after ADDL formation), or to the cells prior to contact with ADDLs. This method (e.g., as previously described) can be employed to detect compounds that modulate ADDL binding to the cell surface. Also, a test compound can be added to the mixture of cells plus ADDLs. This method (e.g., as previously described) can be employed to detect compounds that impact on ADDL-mediated events occurring downstream of ADDL binding to a cell surface protein (e.g., to an ADDL receptor). The specificity of the compounds for acting on an ADDL-mediated downstream effect can be confirmed, for instance, by simply adding the test compound in the absence of any coincubation with ADDLs. Of course, further appropriate controls (e.g., as set forth in the following Examples and as known to those skilled in the art) should be included with all assays.

Similarly, using the methods described herein (e.g., in the Examples), the present invention provides a method for identifying compounds that block formation of the oligomeric structure of the invention, wherein the method desirably comprises:
(a) preparing separate samples of amyloid β protein that either have or have not been mixed with the test compound;
(b) forming the oligomeric structure in the separate samples;
(c) assessing whether any protein assemblies have formed in the separate samples using a method selected from the group consisting of electrophoresis, immunorecognition, and atomic force microscopy; and
(d) comparing the formation of the protein assemblies in the separate samples, which compounds that block formation of the oligomeric structure being identified as resulting in decreased formation of the oligomeric structure in the sample as compared with a sample in which the oligomeric structure is formed in the absence of the test compound.

This information on compounds that modulate (i.e., facilitate or block) formation, activity, or formation and activity, including, but not limited to, binding to a cell surface protein, of the oligomeric structure can be employed in the research and treatment of ADDL-mediated diseases, conditions, or disorders. The methods of the invention can be employed to investigate the activity and neurotoxicity of the ADDLs themselves. For instance, when 20 nL of the ADDL preparation was injected into the hippocampal region of an adult mouse 60-70 minutes prior to the conduct of a long-term potentiation (LTP) experiment (see e.g., Namgung et al. (1995) Brain Research, vol. 689, pp. 85-92), the stimulation phase of the experiment occurred in a manner identical with saline control injections, but the consolidation phase showed a significant, continuing decline in synaptic activity as measured by cell body spike amplitude, over the subsequent 2 hours, compared with control animals, in which synaptic activity remained at a level comparable to that exhibited during the stimulation phase. Analysis of brain slices after the experiment indicated that no cell death had occurred. These results, as well as other described in the following Examples, confirm that ADDL treatment compromised the LTP response. This indicates that ADDLs contribute to the compromised learning and memory observed in Alzheimer's disease by interference with neuronal signaling processes, rather than by the induction of nerve cell death.

Additional information on the effects of ADDLs (either in the presence or absence of test compounds that potentially modulate ADDL formation and/or activity) can be obtained using the further assays according to the invention. For instance, the invention provides a method for assaying the effects of ADDLs that preferably comprises:
(a) administering the oligomeric structure to the hippocampus of an animal;
(b) applying an electrical stimulus; and
(c) measuring the cell body spike amplitude over time to determine the long-term potentiation response.

The method optionally is carried out wherein the long-term potentiation response of the animal is compared to the long-term potentiation response of another animal treated in the same fashion except having saline administered instead of oligomeric structure prior to application of the electrical stimulus. This method further can be employed to identify compounds that modulate (i.e., increase or decrease) the effects of the ADDLs, for instance, by comparing the LTP response in animals administered ADDLs either alone, or, in conjunction with test compounds.

Along these lines, the invention provides a method for identifying compounds that modulate the effects of the ADDL oligomeric structure. The method preferably comprises:
(a) administering either saline or a test compound to the hippocampus of an animal;
(b) applying an electrical stimulus;
(c) measuring the cell body spike amplitude over time to determine the long-term potentiation response; and
(d) comparing the long-term potentiation response of animals having saline administered to the long-term potentiation response of animals having test compound administered.

The method further optionally comprises administering oligomeric structure to the hippocampus either before, along with, or after administering the saline or test compound.

Similarly, the present invention provides a method for identifying compounds that modulate (i.e., either increase or decrease) the neurotoxicity of the ADDL protein assembly, which method comprises:
(a) contacting separate cultures of neuronal cells with the oligomeric structure either in the presence or absence of contacting with the test compound;
(b) measuring the proportion of viable cells in each culture; and
(c) comparing the proportion of viable cells in each culture.

Compounds that block the neurotoxicity of the oligomeric structure are identified, for example, as resulting in an increased proportion of viable cells in the culture as compared to the corresponding culture contacted with the oligomeric structure in the absence of the test compound. Compounds that increase the neurotoxicity of the oligomeric structure are identified, for example, as resulting in a reduced portion of viable cells in the culture as compared to the corresponding culture contacted with the oligomeric structure in the presence of the test compound.

The methods of the invention also can be employed in detecting in test materials the ADDLs (e.g., as part of research, diagnosis, and/or therapy). For instance, ADDLs bring about a rapid morphological change in serum-starved B103 cells, and they also activate Fyn kinase activity in these cells within 30 minutes of ADDL treatment (data not shown). ADDLs also induce rapid complex formation between Fyn and focal adhesion kinase (FAK) (Zhang et al. (1996) Neurosci. Lett., vol. 211, pp. 1-4), and translocating of several phosphorylated proteins and Fyn-Fak complex to a Triton-insoluble fraction (Berg et al. (1997) J. Neurosci. Res., vol. 50, pp. 979-989). This suggests that Fyn and other activated signaling pathways are involved in the neurodegenerative process induced by ADDLs. This has been confirmed by experiments in brain slice cultures from genetically altered mice that lack a functional fyn gene, where addition of ADDLs resulted in no increased neurotoxicity compared to vehicle controls.

Therefore, compounds that block one or more of Fyn's function, or Fyn relocalization, namely by impacting on ADDLs, may be important neuroprotective drugs for Alzheimer's disease. Similarly, when ADDLs are added to cultures of primary astrocytes, the astrocytes become activated and the mRNA for several proteins, including IL-1, inducible nitric oxide synthase, Apo E, Apo J and α1-antichymotrypsin become elevated. These phenomena desirably are employed in accordance with the invention in a method for detecting in a test material the ADDL protein assembly. Such methods optionally comprise:
(a) contacting the test material with an antibody (e.g., the 6E10 antibody or another antibody); and
(b) detecting binding to the oligomeric structure of the antibody.

Similarly, the method desirably can be employed wherein:
(a) the test material is contacted with serum-starved neuroblastoma cells (e.g., B103 neuroblastoma cells); and
(b) morphological changes in the cells are measured by comparing the morphology of the cells against neuroblastoma cells that have not been contacted with the test material.

The method also preferably can be employed wherein:
(a) the test material is contacted with brain slice cultures; and
(b) brain cell death is measured as compared against brain slice cultures that have not been contacted with the test material.

The method further desirably can be conducted wherein:

(a) the test material is contacted with neuroblastoma cells (e.g., B103 neuroblastoma cells); and

(b) increases in fyn kinase activity are measured by comparing fyn kinase activity in the cells against fyn kinase activity in neuroblastoma cells that have not been contacted with said test material.

In particular, Fyn kinase activity can be compared making use of a commercially available kit (e.g., Kit #QIA-28 from Oncogene Research Products, Cambridge, Mass.) or using an assay analogous to that described in Borowski et al. (1994) J. Biochem. (Tokyo), vol. 115, pp. 825-829.

In yet another preferred embodiment of the method of detecting ADDLs in test material, the method desirably comprises:
(a) contacting the test material with cultures of primary astrocytes; and
(b) determining activation of the astrocytes as compared to cultures of primary astrocytes that have not been contacted with the test material.

In a variation of this method, the method optionally comprises:
(a) contacting the test material with cultures of primary astrocytes; and
(b) measuring in the astrocytes increases in the mRNA for proteins selected from the group consisting of interleukin-1, inducible nitric oxide synthase, Apo E, Apo J, and α1-antichymotrypsin by comparing the mRNA levels in the astrocytes against the corresponding mRNA levels in cultures of primary astrocytes that have not been contacted with the test material.

There are, of course, other methods of assay, and further variations of those described above that would be apparent to one skilled in the art, particularly in view of the disclosure herein.

Thus, clearly, the ADDLs according to the present invention have utility in vitro. Such ADDLs can be used inter alia as a research tool in the study of ADDL binding and interaction within cells and in a method of assaying ADDL activity. Similarly, ADDLs, and studies of ADDL formation, activity and modulation can be employed in vivo.

In particular, the compounds identified using the methods of the present invention can be used to treat any one of a number of diseases, disorders, or conditions that result in deficits in cognition or learning (i.e., due to a failure of memory), and/or deficits in memory itself. Such treatment or prevention can be effected by administering compounds that prevent formation and/or activity of the ADDLs, or that modulate (i.e., increase or decrease the activity of, desirably as a consequence of impacting ADDLs) the cell agents with which the ADDLs interact (e.g., so-called “downstream” events). Such compounds having ability to impact ADDLs are referred to herein as “ADDL-modulating compounds”. ADDL-modulating compounds not only can act in a negative fashion, but also, in some cases preferably are employed to increase the formation and/or activity of the ADDLs.

Desirably, when employed in vivo, the method can be employed for protecting an animal against decreases in cognition, learning or memory due to the effects of the ADDL protein assembly. This method comprises administering a compound that blocks the formation or activity of the ADDLs. Similarly, to the extent that deficits in cognition, learning and/or memory accrue due to ADDL formation and/or activity, such deficits can be reversed or restored once the activity (and/or formation) of ADDLs is blocked. The invention thus preferably provides a method for reversing (or restoring) in an animal decreases in cognition, learning or memory due to the effects of an oligomeric structure according to the invention. This method preferably comprises blocking the formation or activity of the ADDLs. The invention thus also desirably provides a method for reversing in a nerve cell decreases in long-term potentiation due to the effects of a soluble non-fibrillar amyloid β oligomeric structure according to the invention (as well as protecting a nerve cell against decrease in long-term potentiation due to the effects of a soluble non-fibrillar amyloid β oligomeric structure), the method comprising contacting the cell with a compound that blocks the formation or activity of the oligomeric structure.

In particular, this method desirably can be applied in the treatment or prevention of a disease, disorder, or condition that manifests as a deficit in cognition, learning and/or memory and which is due to ADDL formation or activity, especially a disease, disorder, or condition selected from the group consisting of Alzheimer's disease, adult Down's syndrome (i.e., over the age of 40 years), and senile dementia.

Also, this method desirably can be applied in the treatment or prevention of early deleterious effects on cellular activity, cognition, learning, and memory that may be apparent prior to the development of the disease, disorder, or condition itself, and which deleterious effects may contribute to the development of, or ultimately constitute the disease, disorder, or condition itself. In particular, the method preferably can be applied in the treatment or prevention of the early malfunction of nerve cells or other brain cells that can result as a consequence of ADDL formation or activity. Similarly, the method preferably can be applied in the treatment or prevention of focal memory deficits (FMD) such as have been described in the literature (see e.g., Linn et al. (1995) Arch. Neurol., vol. 52, pp. 485-490), in the event such FMD are due to ADDL formation or activity. The method further desirably can be employed in the treatment or prevention of ADDL-induced aberrant neuronal signaling, impairment of higher order writing skills (see e.g., Snowdon et al. (1996) JAMA, vol. 275, pp. 528-532) or other higher order cognitive function, decreases in (or absence of) long-term potentiation, that follows as a consequence of ADDL formation or activity.

According to this invention, “ADDL-induced aberrant neuronal signaling” can be measured by a variety of means. For instance, for normal neuronal signaling (as well as observation of a long-term potentiation response), it appears that among other things, Fyn kinase must be activated, Fyn kinase must phosphorylate the NMDA channel (Miyakawa et al. (1997) Science, vol. 278, pp. 698-701; Grant (1996) J. Physiol. Paris, vol. 90, pp. 337-338), and Fyn must be present in the appropriate cellular location (which can be impeded by Fyn-FAK complex formation, for instance, as occurs in certain cytoskeletal reorganizations induced by ADDL). Based on this, ADDL-induced aberrant neuronal signaling (which is a signaling malfunction that is induced by aberrant activation of cellular pathways by ADDLs) and knowledge thereof can be employed in the methods of the invention, such as would be obvious to one skilled in the art. For instance, ADDL-induced aberrant cell signaling can be assessed (e.g., as a consequence of contacting nerve cells with ADDLs, which may further be conducted in the presence or absence of compounds being tested for ADDL-modulating activity) using any of these measures, or such as would be apparent to one skilled in the art, e.g., Fyn kinase activation (or alteration thereof), Fyn-FAK complex formation (or alteration thereof), cytoskeletal reorganization (or alteration thereof), Fyn kinase subcellular localization (or alteration thereof), Fyn kinase phosphorylation of the NMDA channel (or alteration thereof).

Furthermore, instead of using compounds that are identified using the methods of the invention, compounds known to have particular in vitro and in vivo effects can be employed to impact ADDLs in the above-described methods of treatment. Namely, amyloid formation can be (but need not necessarily be) modeled as a two-phase process. In the first phase is initiated the production of amyloid precursor protein (e.g., the amyloid precursor protein of 695 amino acids (Kang et al. (1987) Nature, vol. 325, pp. 733-736) or the 751 amino acid protein (Ponte et al. (1988) Nature, vol. 331, pp. 525-527) each having within their sequence the p amyloid core protein sequence of approximately 4 kDa identified by Glenner et al. (U.S. Pat. No. 4,666,829)). In the second phase occurs amyloid processing and/or deposition into higher molecular weight structures (e.g., fibrils, or any other structure of P amyloid having a molecular weight greater than β amyloid monomer, and including structures that are considerably smaller than plaques and pre-plaques). It is conceivable that some compounds may impact one or both of these phases. For some compounds, a deleterious effect is obtained, but it is not clear whether the locus of inhibition is on protein production, or on amyloid processing and/or deposition.

Thus, relevant to this invention are compounds that act at either the first or second phase, or both phases. In particular, compounds that modulate the second phase have special utility to impact ADDLs and find use in methods of treatment that rely on ADDL modulation. Such compounds that modulate (e.g., block) the deposition of amyloid into higher molecular weight structures include, but are not limited to, compounds that modulate (particularly compounds that impede) the incorporation of β amyloid monomers into higher molecular weight structures, especially fibrils. Accordingly, desirably according to the invention, such compounds that impair incorporation of β amyloid monomers into higher molecular weight structures, particularly compounds that are known to inhibit fibril formation (and thus have been confirmed to inhibit incorporation of β amyloid into higher molecular weight structures), can be employed to exert an inhibitory effect on ADDL formation and/or activity (i.e., by reducing formation of ADDLs), in accordance with the methods of the invention. Of course, it is preferable that prior to such use, the ability of the modulators to impact ADDLs is confirmed, e.g., using the methods of the invention. Such known modulators that desirably can be employed in the present invention are described as follows, however, other similar modulators also can be employed.

In terms of compounds that act at the second phase, PCT International Application WO 96/39834 and Canadian Application 2222690 pertain to novel peptides capable of interacting with a hydrophobic structural determinant on a protein or peptide for amyloid or amyloid-like deposit formation, thereby inhibiting and structurally blocking the abnormal folding of proteins and peptides into amyloid and amyloid-like deposits. In particular, the '834 application pertains to inhibitory peptides comprising a sequence of from about 3 to about 15 amino acid residues and having a hydrophobic cluster of at least three amino acids, wherein at least one of the residues is a P-sheet blocking amino acid residue selected from Pro, Gly, Asn, and H is, and the inhibitory peptide is capable of associating with a structural determinant on the protein or peptide to structurally block and inhibit the abnormal filing into amyloid or amyloid-like deposits.

PCT International Application WO 95/09838 pertains to a series of peptidergic compounds and their administration to patients to prevent abnormal deposition of β amyloid peptide.

PCT International Application WO 98/08868 pertains to peptides that modulate natural β amyloid peptide aggregation. These peptide modulators comprise three to five D-amino acid residues and include at least two D-amino acid residues selected from the group consisting of D-leucine, D-phenylalanine, and D-valine.

Similarly, PCT International Application WO 96/28471 pertains to an amyloid modulator compound that comprises an amyloidogenic protein or peptide fragment thereof (e.g., transthyretin, prion protein, islet amyloid polypeptide, atrial natriuretic factor, kappa light chain, lambda light chain, amyloid A, procalcitonin, cystatin C, (β2-microglobulin, ApoA-1, gelsolin, procalcitonin, calcitonin, fibrinogen, and lysozyme) coupled directly or indirectly to at least one modifying group (e.g., comprises a cyclic, heterocyclic, or polycyclic group, contains a cis-decalin group, contains a cholanyl structure, is a cholyl group, comprises a biotin-containing group, a fluorescein-containing group, etc.) such that the compound modulates the aggregation of natural amyloid proteins or peptides when contacted with these natural amyloidogenic proteins or peptides.

Also, PCT International Application WO 97/21728 pertains to peptides that incorporate the Lys-Leu-Val-Phe-Phe (KVLFF) sequence of amyloid β that is necessary for polymerization to occur. Peptides that incorporate this sequence bind to amyloid β and are capable of blocking fibril formation.

In terms of non-peptide agents, PCT International Application WO 97/16191 pertains to an agent for inhibiting the aggregation of amyloid protein in animals by administering a 9-acridinone compound having the formula:

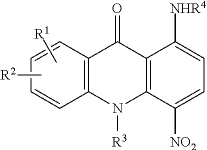

wherein R¹ and R² are hydrogen, halo, nitro, amino, hydroxy, trifluoromethyl, alkyl, alkoxy, and alkylhio; R³ is hydrogen or alkyl; and R⁴ is allylene-NR⁵, R⁶, wherein R⁵ and R⁶ are independently hydrogen, C₁-C₄ alkyl, or taken together with the nitrogen to which they are attached are piperidyl or pyrrolidinyl, and the pharmaceutically acceptable salts thereof. The disclosed compounds previously were identified as antibacterial and antitumor agents (U.S. Pat. No. 4,626,540) and as antitumor agents (Cholody et al. (1990) J. Med. Chem., vol. 33, pp. 49-52; Cholody et al. (1992) J. Med. Chem., vol. 35, pp. 378-382).

PCT International Application WO 97/16194 pertains to an agent for inhibiting the aggregation of amyloid protein in animals by administering a naphthylazo compound having the formula:

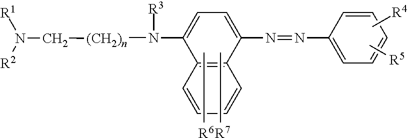

wherein R¹ and R² independently are hydrogen, alkyl, substituted alkyl, or a complete heterocyclic ring, R³ is hydrogen or alkyl, R⁴, R⁵, R⁶, and R⁷ are substituent groups including, but not limited to hydrogen, halo, alkyl, and alkoxy.

Japanese Patent 9095444 pertains to an agent for inhibiting the agglomeration and/or deposition of amyloid protein wherein this agent contains a thionaphthalene derivative of the formula:

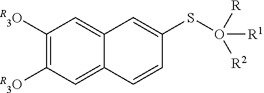

wherein R is a 1-5 carbon alkyl substituted with OH or COOR⁴ (optionally substituted by aryl, heterocyclyl, COR⁵, CONHR⁶, or cyano; R⁴ is H or 1-10 carbon alkyl, 3-10 carbon alkenyl, 3-10 carbon cyclic alkyl (all optionally substituted); R⁵ and R⁶ are optionally substituted aryl or heterocyclyl; R¹ and R² are H, 1-5 carbon alkyl or phenyl; R³ is hydrogen, 1-5 carbon alkyl or COR⁷; R⁷ is OR′, —R″ or —N(R′″)₂; R′, R″, R′″ is 1-4 carbon alkyl.

Japanese Patent 7309760 and PCT International Application WO 95/11248 pertain to inhibitors of coagulation and/or deposition of amyloid β protein which are particular rifamycin derivatives. Japanese Patent 7309759 pertains to inhibitors of coagulation and/or deposition of amyloid β protein which are particular rifamycin SV derivatives. Japanese Patent 7304675 pertains to inhibitors of agglutination and/or precipitation of amyloid β protein which are particular 3-homopiperazinyl-rifamycin derivatives.

Japanese Patent 7247214 pertains to pyridine derivatives and that salts or prodrugs that can be employed as inhibitors of β-amyloid formation or deposition.

U.S. Pat. No. 5,427,931 pertains to a method for inhibiting deposition of amyloid plaques in a mammal that comprises the administration to the mammal of an effective plaque-deposition inhibiting amount of protease nexin-2, or a fragment or analog thereof.

In terms of compounds that may act at either the first or second phase (i.e., locus of action is undefined), PCT International Application WO 96/25161 pertains to a pharmaceutical composition for inhibiting production or secretion of amyloid β protein, which comprises a compound having the formula:

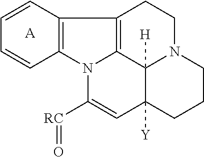

wherein ring A is an optionally substituted benzene ring, R represents OR¹,

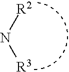

or SR¹, wherein R¹, R² and R³ are the same or different and each is selected from a hydrogen atom, an optionally substituted hydrocarbon group or R² and R³, taken together with the adjacent nitrogen atom, form an optionally substituted nitrogen-containing heterocyclic group, and Y is an optionally substituted alkyl group, or a pharmaceutically acceptable salt thereof, if necessary, with a pharmaceutically acceptable excipient, carrier or diluent. Of course, it is preferred that these and other known modulators (e.g., of the first phase or the second phase) are employed according to the invention. It also is preferred that gossypol and gossypol derivatives be employed. Furthermore, it is contemplated that modulators are employed that have ability to impact ADDL activity (e.g., PCT International Applications WO 93/15112 and 97/26913).

Also, the ADDLs themselves may be applied in treatment. It has been discovered that these novel assemblies described herein have numerous unexpected effects on cells that conceivably can be applied for therapy. For instance, ADDLs activate endothelial cells, which endothelial cells are known, among other things to interact with vascular cells. Along these lines, ADDLs could be employed, for instance, in wound healing. Also, by way of example, Botulinum Toxin Type A (BoTox) is a neuromuscular junction blocking agent produced by the bacterium Clostridium botulinum that acts by blocking the release of the neurotransmitter acetylcholine. Botox has proven beneficial in the treatment of disabling muscle spasms, including dystonia. ADDLs themselves theoretically could be applied to either command neural cell function or, to selectively destroy targeted neural cells (e.g., in cases of cancer, for instance of the central nervous system, particularly brain). ADDLs appear further advantageous in this regard given that they have very early effects on cells, and given that their effect on cells (apart from their cell killing effect) appears to be reversible.

As discussed above, the ADDL-modulating compounds of the present invention, compounds known to impact incorporation of amyloid β into higher molecular weight structures, as well as ADDLs themselves, can be employed to contact cells either in vitro or in vivo. According to the invention, a cell can be any cell, and, preferably, is a eukaryotic cell. A eukaryotic cell is a cell typically that possesses at some stage of its life a nucleus surrounded by a nuclear membrane. Preferably the eukaryotic cell is of a multicellular species (e.g., as opposed to a unicellular yeast cell), and, even more preferably, is a mammalian (optionally human) cell. However, the method also can be effectively carried out using a wide variety of different cell types such as avian cells, and mammalian cells including but not limited to rodent, primate (such as chimpanzee, monkey, ape, gorilla, orangutan, or gibbon), feline, canine, ungulate (such as ruminant or swine), as well as, in particular, human cells. Preferred cell types are cells formed in the brain, including neural cells and glial cells. An especially preferred cell type according to the invention is a neural cell (either normal or aberrant, e.g., transformed or cancerous). When employed in tissue culture, desirably the neural cell is a neuroblastoma cell.

A cell can be present as a single entity, or can be part of a larger collection of cells. Such a “larger collection of cells” can comprise, for instance, a cell culture (either mixed or pure), a tissue (e.g., neural or other tissue), an organ (e.g., brain or other organs), an organ system (e.g., nervous system or other organ system), or an organism (e.g., mammal, or the like). Preferably, the organs/tissues/cells of interest in the context of the invention are of the central nervous system (e.g., are neural cells).

Also, according to the invention “contacting” comprises any means by which these agents physically touch a cell. The method is not dependent on any particular means of introduction and is not to be so construed. Means of introduction are well known to those skilled in the art, and also are exemplified herein. Accordingly, introduction can be effected, for instance, either in vitro (e.g., in an ex vivo type method of therapy or in tissue culture studies) or in vivo. Other methods also are available and are known to those skilled in the art.

Such “contacting” can be done by any means known to those skilled in the art, and described herein, by which the apparent touching or mutual tangency of the ADDLs and ADDL-modulating compounds and the cell can be effected. For instance, contacting can be done by mixing these elements in a small volume of the same solution. Optionally, the elements further can be covalently joined, e.g., by chemical means known to those skilled in the art, or other means, or preferably can be linked by means of noncovalent interactions (e.g., ionic bonds, hydrogen bonds, Van der Waals forces, and/or nonpolar interactions). In comparison, the cell to be affected and the ADDL or ADDL-modulating compound need not necessarily be brought into contact in a small volume, as, for instance, in cases where the ADDL or ADDL-modulating compound is administered to a host, and the complex travels by the bloodstream or other body fluid such as cerebrospinal fluid to the cell with which it binds. The contacting of the cell with a ADDL or ADDL-modulating compound sometimes is done either before, along with, or after another compound of interest is administered. Desirably this contacting is done such that there is at least some amount of time wherein the coadministered agents concurrently exert their effects on a cell or on the ADDL.

One skilled in the art will appreciate that suitable methods of administering an agent (e.g., an ADDL or ADDL-modulating compound) of the present invention to an animal for purposes of therapy and/or diagnosis, research or study are available, and, although more than one route can be used for administration, a particular route can provide a more immediate and more effective reaction than another route. Pharmaceutically acceptable excipients also are well-known to those who are skilled in the art, and are readily available. The choice of excipient will be determined in part by the particular method used to administer the agent. Accordingly, there is a wide variety of suitable formulations for use in the context of the present invention. The following methods and excipients are merely exemplary and are in no way limiting.

Formulations suitable for oral administration can consist of (a) liquid solutions, such as an effective amount of the compound dissolved in diluents, such as water, saline, or orange juice; (b) capsules, sachets or tablets, each containing a predetermined amount of the active ingredient, as solids or granules; (c) suspensions in an appropriate liquid; and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, emulsions, gels, and the like containing, in addition to the active ingredient, such excipients as are known in the art.

An agent of the present invention, alone or in combination with other suitable ingredients, can be made into aerosol formulations to be administered via inhalation. These aerosol formulations can be placed into pressurized acceptable propellants, such as dichlorodifluoromethane, propane, nitrogen, and the like. They also can be formulated as pharmaceuticals for non-pressured preparations such as in a nebulizer or an atomizer.

Formulations suitable for parenteral administration are preferred according to the invention and include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

The dose administered to an animal, particularly a human, in the context of the present invention will vary with the agent of interest, the composition employed, the method of administration, and the particular site and organism being treated. However, preferably a dose corresponding to an effective amount of an agent (e.g., an ADDL or ADDL-modulating compound according to the invention) is employed. An “effective amount” is one that is sufficient to produce the desired effect in a host, which can be monitored using several end-points known to those skilled in the art. Some examples of desired effects include, but are not limited to, an effect on learning, memory, LTP response, neurotoxicity, ADDL formation, ADDL cell surface protein (e.g., receptor) binding, antibody binding, cell morphological changes, Fyn kinase activity, astrocyte activation, and changes in mRNA levels for proteins such as interleukin-1, inducible nitric oxide synthase, ApoE, ApoJ, and α1-antichymotrypsin. These methods described are by no means all-inclusive, and further methods to suit the specific application will be apparent to the ordinary skilled artisan.

Moreover, with particular applications (e.g., either in vitro or in vivo) the actual dose and schedule of administration of ADDLs or ADDL-modulating compounds can vary depending on whether the composition is administered in combination with other pharmaceutical compositions, or depending on interindividual differences in pharmacokinetics, drug disposition, and metabolism. Similarly, amounts can vary in in vitro applications depending on the particular cell type utilized or the means or solution by which the ADDL or ADDL-modulating compound is transferred to culture. One skilled in the art easily can make any necessary adjustments in accordance with the requirements of the particular situation.

With use of certain compounds, it can be desirable or even necessary to introduce the compounds (i.e., agents) as pharmaceutical compositions directly or indirectly into the brain. Direct techniques include, but are not limited to, the placement of a drug delivery catheter into the ventricular system of the host, thereby bypassing the blood-brain barrier. Indirect techniques include, but are not limited to, the formulation of the compositions to convert hydrophilic drugs into lipid-soluble drugs using techniques known in the art (e.g., by blocking the hydroxyl, carboxyl, and primary amine groups present on the drug) which render the drug able to cross the blood-brain barrier. Furthermore, the delivery of hydrophilic drugs can be improved, for instance, by intra-arterial infusion of hypertonic solutions (or other solutions) which transiently open the blood brain barrier.

The following are incorporated by reference to the extent that they are not contradictory to the invention disclosed and claimed herein:
1) European Patent App. No. EP 01172378 “Human beta-amyloid antibody and its use for the treatment of Alzheimer's disease,” which discloses “a human anti-Abeta-amyloid antibody derived from a human IgG-containing body fluid by Abeta affinity chromatography and its use for the diagnosis or treatment of amyloid-associated disease, especially Alzheimer's disease, and primary and secondary amyloidoses are claimed. The use of an IgG-containing fluid for treating amyloid-associated diseases, and pharmaceutical compositions comprising an anti-Abeta-amyloid antibody are also claimed. The treatment of Alzheimer's disease by infusion of human IgG immunoglobulins or anti-Abeta antibodies from human IgG is described. Administration of immunoglobulins (Octagam, Polyglobulin) iv to 4 patients with neurological diseases decreased the amount of beta-amyloid in the CSF from 1835 ng/l before treatment to 1376 ng/14 weeks after treatment.”
2) International Publication No. WO 00/071671 “Novel mutant genes and their use in models of amyloid-associated neurodegenerative disease,” which discloses “the mutant amyloid precursor polypeptides, ABriPP and ADanPP, and their amyloid peptides, ABri and ADan, which are associated with Familial British Dementia (FBD) and Familial Danish Dementia (FDD), respectively, and their use for inhibiting cerebral amyloidosis and for screening potential therapeutic agents for these dementias are claimed. Polynucleotides encoding the polypeptides and peptides, expression vectors and transgenic animals with DNA encoding the amyloid precursor polypeptides are also claimed. Specific antibodies, immunoassays and vaccine compositions capable of inducing a specific immune response against a mutant epitope of ABri or ADan are additionally claimed. Transgenic mice provided by this invention are stated to be of particular value in studies of neurodegenerative conditions such as Alzheimer's disease. Amyloid fibrils were isolated from leptomeningeal and parenchymal deposits of a patient with FBD and a patient with FDD. Nucleic acid and amino acid sequences were determined and antibodies were raised. The amyloid deposited in both conditions originated from the same precursor protein, carrying different genetic defects.”
3) International Publication No. WO 00/072876 “Prevention and treatment of amyloidogenic disease,” which discloses “a composition which comprises an agent capable of inducing an immune response against an amyloid component in a patient, and a pharmaceutical excipient, is claimed. Its use for the treatment and prevention of disorders characterized by amyloid deposition and a method of determining the prognosis of a patient undergoing treatment for an amyloid disorder are also claimed. In order to test the efficacy of Abeta against Alzheimer's disease, Abeta42 peptide was administered to transgenic mice overexpressing APP having a mutation at position 717 that predisposes them to develop Alzheimer's-like neuropathology. The mice were injected with either Abeta42, SAP peptides or PBS. Mice were monitored and sacrificed at 13 months. The mice given aggregated Abeta42 developed a high antibody titer. Seven out of nine mice treated with Abeta42 had no detectable amyloid in their brains. The results are presented in a figure.”
4) International Publication No. WO 00/072880 “Prevention and treatment of amyloidogenic disease,” which discloses “a method of preventing or treating a disease associated with amyloid deposits of Abeta, such as Alzheimer's disease, Down's syndrome or mild cognitive impairement by administering an antibody, and optionally a second antibody, of human isotype IgG1, IgG2, IgG3, or IgG4 that binds to an epitope within residues 1-10 of A, or a polynucleotide sequence encoding the antibody, or a peptide comprising an N-terminal segment of at least residues 1-5 of Abeta, amongst others, is claimed. The method is claimed to induce a clearing Fc receptor mediated phagocytosis response against the amyloid deposit. Screening methods to detect amyloid deposits and for identifying antibodies having activity in clearing an antigen, are also claimed. The prophylactic efficacy of Abeta against Alzheimer's disease was tested by the administration of Abeta1-42 (AN1792) peptide to transgenic mice overexpressing APP having a mutation at position 717 that predisposes them to develop Alzheimer's-like neuropathology (PDAPP mice). It was found that Abeta1-42 injections are highly effective in the prevention of deposition or clearance of human Abeta from brain tissue, and elimination of subsequent neuronal and inflammatory degenerative changes. The effects of AN1792 in PDAPP mice include a 72% reduction in cortical Abeta levels, a significant reduction (84%) of the neuritic plaque burden in the frontal cortex and suppressed development of astrocytosis. It was also shown that immunization with a synthetic Abeta protein generates antibodies that bind in vivo to the Abeta in amyloid plaques.”
   5) International Publication No. WO 00/077178 “Immunological control of beta-amyloid levels in vivo for the treatment of Alzheimer's disease,” which discloses “an antibody which catalyzes hydrolysis of specified amide linkages within beta-amyloid (beta-A) is claimed. An antibody with this activity and capable of crossing the blood brain barrier, is also claimed. Methods for sequestering free beta-A in the bloodstream, and reducing levels of beta-A in the brain of an animal by immunizing with a beta-A antigen or by administering specific antibodies are also claimed. Methods for preventing amyloid plaque formation, reducing circulating beta-A levels and disaggregating amyloid plaques by providing an antigen epitope from endogenous beta-A or one that mimics a hydrolysis transition state, or by administering antibodies are further claimed. Methods for generating such antibodies are additionally claimed. Mice immunized with three peptide antigens from beta-A were shown by ELISA to produce antibodies specific for different epitopes and full length beta-A. An alum-based beta-A peptide vaccine used to immunize cynomolgus monkeys also generated a strong immune response to the peptide. Anti-beta-A transition state antibodies were generated, and it is stated that they may be able to force native beta-A peptide into a transition state conformation, allowing cleavage to potentially less harmful shorter peptides. Anti-beta-A antibodies were linked to antitransferrin receptor antibodies (anti-TfR) as vectors for delivery into the brain. A bispecific antibody was shown to attach to TfR-bearing mouse cell membranes and bind [125I]-beta-A. When [125I]-beta-A was administered to live mice, brain levels were shown to increase between 1 and 6 h and to decrease between 24 and 48 h. The possibilities of using smaller modified bispecific agents for more efficient entry to the brain, and to avoid detrimental complement fixation, are discussed.”
   6) International Publication No. WO-00139796 “Vaccine for the prevention and treatment of Alzheimer's and amyloid-related diseases,” which discloses “novel methods for preventing or treating an amyloid-related disease in a subject comprising administering an antigenic amount of an all-D peptide are claimed. A vaccine for preventing or treating an amyloid-related disease in a subject comprising an antibody raised against an antigenic amount of an all-D peptide, which interacts with at least one region of an amyloid protein and prevents fibrillogenesis is also claimed. A vaccine for preventing or treating an amyloid-related disease in a subject comprising an antigenic amount of an all-D peptide, which interacts with at least one region of an amyloid protein, is additionally claimed. The use of the vaccines for preventing or treating an amyloid-related disease or manufacture of a medicament for preventing or treating an amyloid-related disease is further claimed. Antibodies raised to all-D peptides in rabbits had about 5-fold higher anti-fibrillogenic activity than anti-all-L peptide antibodies and results are shown in two figures. It was shown that the anti-KLVFFA antibody recognized only non-aggregated form of Abeta and did not bind to plaques in brain sections.”
   7) International Publication No. WO00/142306 “Immunogenic chimeric peptides and antibodies to these useful for immunization against amyloid-beta peptides associated with Alzheimer's disease,” which discloses “chimeric peptides with an end-specific B-cell epitope from a naturally-occurring internal peptide cleavage product of a precursor or mature protein as a free N- or C-terminus fused to a different T-helper cell epitope, with or without spacer residues, are claimed. The T-helper cell epitope may be derived from tetanus toxin, pertussis toxin, diphtheria toxin, measles virus F protein, hepatitis B surface antigen, Chlamydia trachomatis major outer membrane protein, Plasmodium falciparum circumsporozoite, Schistosoma mansonii triose phosphate isomerase, or E coli TraT. Immunizing compositions and methods for immunization against the free N-terminus or free C-terminus of an internal self peptide cleavage product are also claimed. The internal self peptide cleavage product may be an amyloid-beta peptide. Antigen-binding portions of an antibody specific for the chimeric peptides and the use of these for passive immunization are additionally claimed. The antibody may be one raised against an amyloid-beta peptide derived from the cleavage of beta-amyloid precursor protein (betaAPP). A schematic representation of the betaAPP and the products of secretase cleavage is given. The partial amino acid sequence of betaAPP from which amyloid-beta peptides are derived is given. No other original biological data are presented.”
   8) International Publication No. WO 00/153457 “Vaccines against neuro-degenerative disorders,” which discloses a pharmaceutical composition comprising an antigenic molecule associated with a neurodegenerative disorder, which is not beta-amyloid, is claimed. The composition is specifically claimed where the antigenic molecule is an oligomeric Abeta complex, ApoE4-Abeta complex, tau protein, alpha-synuclein, a mutant amyloid precursor, presenilin, or a prion protein and where it further comprises an adjuvant such as an immunostimulatory molecule or microparticulate adjuvant. Pharmaceutical compositions for treatment or prevention of neurodegenerative diseases comprising recombinant human cells transformed with the polynucleotides encoding an antigenic molecule, carrier protein or fusion protein are also claimed. Methods for eliciting an immune response against an antigen by administering an antigenic molecule and compositions of antigen presenting cells sensitized in vitro with a second antigenic molecule are further claimed. Various proteins are stated to be sources of antigenic molecules associated with neurodegenerative disorders, including alipoprotein E4, amyloid precursor protein, tau protein and prion proteins. Various methods for recombinant production and purification of the antigens are described, and potential uses in the treatment and prevention of neurodegenerative disorders are discussed. Methods for treatment, including combination with adoptive immunotherapy, sensitization of macrophages and antigen presenting cells with antigens, and for formulation of antigens vaccines and assaying immunogenicity and efficacy are also discussed.”
   9) International Publication No. WO 00/162284 “A vaccine for treatment of Alzheimer's disease,” which discloses “proteins which can be used to vaccinate an individual against amyloidogenic polypeptides are claimed. It is claimed that down regulation of amyloid protein can be achieved by immunising with an amyloidogenic polypeptide containing a B-cell epitope or a T-cell epitope. Modifications which target the modified molecule to an antigen presenting cell are claimed. It is claimed that the polypeptide used as the vaccine can be modified by coupling to palmitoyl or farnesyl groups or the polypeptide can be modified by coupling to a polysaccharide via an amide linkage. The polypeptide vaccine and the T-cell epitope can be separately bound to the polysaccharide. At least 12 administrations per year are claimed for use in reducing the amount of amyloid protein and giving effective treatment of Alzheimer's disease. 35 Constructs containing various portions of the APP protein together with B-cell epitopes and the T-cell epitopes P30 and P2. One such polypeptide construct was expressed in Escherichia coli and purified from inclusion bodies and refolded. Transgenic mice containing human APP were immunised with a synthetic peptide comprising residues 673-714 of Abeta-42 or the protein from one of the 35 constructs. High antibody titres were seen after 4 immunizations with the Abeta-42 protein. The synthesis of an Abeta peptide copolymer vaccine is also described which contains P2 and P30 peptides as well as the Abeta-42 peptide.”
   10) International Publication No. WO 00/162801 “Humanized antibodies that sequester Abeta peptide,” which discloses “a humanized antibody that specifically binds an epitope contained within positions 13 to 28 of Abeta and sequesters Abeta peptide from its bound, circulating form in blood, and alters clearance of soluble and bound forms of Abeta in central nervous systems and plasma is claimed. A nucleic acid, expression vector and transfected cell for the recombinant production of the antibody or fragment of it are also claimed. It is claimed that administration of the humanized antibody can be used to reduce or inhibit the formation of amyloid plaques or the effects of toxic soluble Abeta species in humans, which is useful in the treatment of Alzheimer's disease, Down syndrome, and cerebral amyloid angiopathy. It was shown that in human CSF, only Mab 266 and Mab 4G8 were able to sequester Abeta peptide. Furthermore sequestration of Abeta was not perturbed by anti-apoE antibodies. Sequestration of Abeta peptide in vivo demonstrated that the peptide is withdrawn from the brain parenchyma into the CSF by the presence of Mab 266 in the bloodstream. The affinity of humanized 266 for Abeta1-42 was found to be 4 μM.”
   11) International Publication No. WO 00/190182 “Synthetic immunogenic but non-amyloidogenic peptides homologous to amyloid beta for induction of an immune response to amyloid beta and amyloid deposits,” which discloses “an isolated peptide and a conjugate of the peptide cross-linked to a polymer molecule such as a promiscuous T-helper cell epitope are claimed. An immunizing composition comprising the isolated peptide or conjugate and a pharmaceutically acceptable carrier is also claimed. A molecule that includes the antigen-binding portion of an antibody raised against the peptide such as a monoclonal, chimeric or humanized antibody and a pharmaceutical composition comprising the molecule and a pharmaceutically acceptable carrier are further claimed. A method is also claimed for reducing the formation of amyloid fibrils and deposits comprising administering the molecule. The prototype peptide, K6Abeta1-30-NH2 was shown to not form fibrils for at least 15 days. K6Abeta1-30-NH2 was shown to have no effect on human neuroblastoma cell viability after 2 days and was slightly trophic after 6 days. Mice vaccinated with K6Abeta1-30-NH2 had 81% and 89% reduction in cortical and hippocampal amyloid burden, respectively compared to controls. Sequence listings are disclosed.”
   12) International Publication No. WO 00/200245 “Neurotoxic oligomers and their potential value in treating Alzheimer's disease and other disorders,” which discloses “the use of an immunizing-effective dose of one or more tyrosine crosslinked compounds for the prophylaxis, treatment or amelioration of a disease characterized by pathological aggregation and accumulation of a protein associated with oxidative damage and formation of tyrosine crosslinks is claimed. The disease may be Alzheimer's disease, amyotrophic lateral sclerosis, cataract, Parkinson's disease, Creutzfeldt-Jakob disease, Huntington's chorea, dementia with Lewy body formation, multiple system atrophy, Hallervorden-Spatz disease or diffuse Lewy body disease. The compound may be coupled to a carrier protein which is itself immunogenic. The use of antibodies and antibody fragments in these diseases and a diagnostic method based on the assay of a sample of a biological fluid from a patient for the presence of a molecule containing tyrosine crosslinks are also claimed. The method of inducing dityrosine crosslinking and the structure of the polypeptide being crosslinked were shown to be critical in the recognition of dityrosine by an antibody. Methods of determining the effect of immunization with dityrosine on amyloid-beta deposits in transgenic animals are described and the effects of treatment with antibodies against dityrosine in mice are discussed.”
   13) International Publication No. WO 00/221141 “Methods and compositions for treating diseases associated with amyloidosis,” which discloses “a composition comprising a fusion protein comprising an antibody or antibody fragment, and at least one or more segments comprising portions or fragments of transferrin, which is capable of crossing the blood brain barrier is claimed. A molecular construct and an expression vector for the production of the fusion protein are also claimed. It is claimed that the fusion protein is capable of altering amyloid deposition in a human. A further composition is claimed comprising at least one modified peptide, fragment or protein anchored in a liposome, where the peptide is a palmitoylated beta-amyloid1-16 peptide. Both compositions are claimed to be useful in treating amyloid-associated diseases. Transgenic NOBRA mice that presented beta-amyloid plaques on their pancreas were immunized with six ip inoculations at 2-week intervals with 200 mul of a palmitoylated beta-amyloid1-16 peptide-liposome/alum suspension. An ELISA was used to assay blood collected from the mice for anti-beta-amyloid antibodies; in 1:5000 dilutions of the sera the OD45 was 10-fold higher than in controls. A histological study of thioflavin-stained sections of pancreases from the vaccinated NOBRA mice showed that the vaccination either disintegrated beta-amyloid plaques or reversed their deposition. Quantitative evaluation of the average fluorescence intensity in each stained section indicated that the pancreas sections from the NOBRA vaccinated mice showed <25% of the high intensity fluorescence of the same mice unvaccinated.”
   14) International Publication No. WO 02/060481 “Use low-level antibody treatment of diseases associated with toxins or infectious agents,” which discloses “a method of treating a disease associated with the presence of a toxin or infectious agent by administering an antibody specific for the toxin or infectious agent in a dose of <0.1 mg/day is claimed. The antibody may be monoclonal and administration may be po, by oral drench, sublingually, or by injection. The disease may be cancer, pulmonary infection, Alzheimer's disease, diabetes, Crohn's disease or rheumatoid arthritis. Pharmaceutical compositions are also claimed. Examples are given of the treatment of attention deficit syndrome and of multiple sclerosis with low doses of antirubeola antibody and the treatment of juvenile rheumatoid arthritis with antibodies specific for Klebsiella pneumoniae. The use of anti-amyloid beta antibodies in Alzheimer's disease and in senile dogs is also described.”
   15) International Publication No. WO 96/25435 “Monoclonal antibody specific for betaA4 peptide,” which discloses “a novel monoclonal antibody that binds the betaA4 peptide derived from Amyloid Precursor Protein, is claimed. The invention is claimed to be potentially useful for diagnosis and treatment of Alzheimers disease. Prior art has been shown to be less specific in binding.

Release of the betaA4 peptide is symptomatic of Alzheimers disease, with massive beta-amyloid plaque deposits found in brain regions of Alzheimers disease patients. The monoclonal antibody is specific for the free C-terminus of betaA4 peptide (betaA4′1-42′). The antibody binds to diffuse and fibrillar amyloid, neurofibrillary tangles and vascular amyloid. The administration of the monoclonal antibody is claimed to prevent the aggregation of the betaA4 peptide, thus limiting disease. The betaA4 peptide was expressed heterologously and monoclonal antibodies were raised in Balb/c mice. The best cell line was selected and the antibody was demonstrated to bind at high affinity and high specificity to amyloid plaque cores and other amyloid deposits. The betaA4 1-42 peptide antibody was shown to bind effectively, whereas the betaA4 1-43 peptide antibody did not.”
16) International Publication No. WO 98/05350 “Materials and methods for treatment of plaquing diseases,” which discloses “methods and compositions for alleviating symptoms of diseases associated with amyloid and arterial plaque formation are claimed. The compositions comprise an amyloid protein and/or thimerosal for use in Alzheimer's disease, Parkinson's disease, atherosclerosis, hypertension, herpes and chronic fatigue syndrome.

Thimerosal is a preservative in commercially available influenza virus vaccines. Six patients with a history of Alzheimer's disease were given four daily sublingual doses of 10-4 mg of amyloid beta protein for 3 to 4 months showed increases in score on the mini mental state examinination during treatment. Five patients with atherosclerosis given amyloid beta protein and thimerosal showed reductions in blood pressure. Thimerosal alone assessed in a double blind trial in 16 patients with chronic fatigue syndrome resulting in significant improvements in severity. Antiherpes activity of thimerosal with and without influenza vaccine was confirmed in in vitro studies and in seven patients. The specified composition comprises 10-10 to 10-2 mg amyloid beta protein and/or 0.05 to 500 mug thimerosal and administered at a dose of 0.05 ml sublingually per patient and is specifically claimed for this use.”
17) International Publication No. WO 98/44955 “Recombinant antibodies specific for beta-amyloid ends, DNA encoding them and methods of use thereof,” which discloses “a method for preventing or inhibiting the progression of Alzheimer's disease is claimed. The method comprises the administration of a nucleic acid sequence encoding an antibody end-specific for the C- or N-terminus of the beta-amyloid peptide. The antibody encoding sequence is linked to a promoter suitable for expression in the central nervous system. This technology is designed to prevent the accumulation of beta-amyloid peptides and thus prevent the aggregation processes which lead to amyloid deposits in the brain. Use of the beta-APP promoter is specifically claimed. The production of beta-amyloid peptide end specific monoclonal antibodies using standard hybridoma techniques, using terminal peptide sequences conjugated to bovine serum albumin, is described. The purified antibodies were shown to be effective in vitro in preventing the beta-amyloid peptide aggregation and beta-amyloid peptide induced neurotoxicity in mouse brain cells. The cloning of the immunoglobulin variable domains and the construction of recombinant adeno-associated viral vectors for regional expression of the Fv regions in the brain is also described.”
18) International Publication No. WO 99/27944 “Prevention and treatment of amyloidogenic disease,” which discloses “a method for preventing or treating an amyloidogenic disease and a pharmaceutical composition for use in this method are claimed. The method comprises administering an agent which induces an immune response against amyloid protein, especially aggregated beta-amyloid (Abeta). The composition is claimed to comprise Abeta, an active fragment of it, or nucleic acid encoding the protein. An assay to determine efficacy of Abeta1-42 in treating Alzheimer's disease was performed in PDAPP mice with brain amyloid plaques. Cortical amyloid burden was reduced by 96% after 15 months and 99% after 18 months compared to control. Effects of different adjuvants are further exemplifed.”
19) International Publication No. WO 99/58564 “Mutant peptides of the beta-amyloid precursor protein and the ubiquitin-B protein for use in the prevention of Alzheimers and Down syndrome,” which discloses “novel peptide mutants of the beta-amyloid precursor protein or the ubiquitin-B protein, pharmaceutical compositions comprising them, DNA sequences encoding them, and plasmids or vectors comprising such DNA sequences, are claimed. These peptides contain frameshift mutations of the proteins and are claimed for use to treat or as a vaccine against Alzheimer's disease or Down syndrome. The method of vaccination claimed consists of administering the peptide until the production of specific T-cell immunity to the mutant peptides has developed. Methods of administering the peptides are disclosed including the use of other cytokines and growth factors such as IL-2, IL-12 and GM-CSF to improve the response of the immune system, but no biological data are presented. The peptides were synthesized using continuous flow solid phase peptide synthesis. Fmoc-amino acids were activated for coupling as pentafluorophenyl esters. A 20% piperidine in DMF solution was then used for the selective removal of Fmoc after each coupling. The peptides were purified and analyzed by reverse phase HPLC and the identity of the peptides confirmed using electro-spray mass spectroscopy. Ten peptides comprising 5 to 28 amino acids including the specified compound, Asn-Val-Pro-Gly-His-Glu-Arg-Met-Gly-Arg-Gly-Arg-Thr-Ser-Ser-Lys-Glu-Leu-Ala, are specifically claimed.”

The foregoing descriptions and citations (as well as those which follow) are exemplary only. Other applications of the method and constituents of the present invention will be apparent to one skilled in the art. Thus, the following examples further illustrate the present invention, but should not be construed to limit the scope of the claimed invention in any way.

### Example 1

### Preparation of Amyloid β-Oligomers

According to the invention, ADDLs were prepared by dissolving 1 mg of solid amyloid β1-42 (e.g., synthesized as described in Lambert et al. (1994) J. Neurosci. Res., vol. 39, pp. 377-395) in 44 μL of anhydrous DMSO. This 5 mM solution then was diluted into cold (4° C.) F12 media (Gibco BRL, Life Technologies, Gaithersburg, Md.)) to a total volume of 2.20 mL (50-fold dilution), and vortexed for about 30 seconds. The mixture was allowed to incubate at from about 0° C. to about 8° C. for about 24 hours, followed by centrifugation at 14,000 g for about 10 minutes at about 4° C. The supernatant was diluted by factors of 1:10 to 1:10,000 into the particular defined medium, prior to incubation with brain slice cultures, cell cultures or binding protein preparations. In general, however, ADDLs were formed at a concentration of Aβ protein of 100 μM. Typically, the highest concentration used for experiments is 10 μM and, in some cases, ADDLs (measured as initial Aβ concentration) were diluted (e.g., in cell culture media) to 1 nM. For analysis by atomic force microscopy (AFM), a 20 μL aliquot of the 1:100 dilution was applied to the surface of a freshly cleaved mica disk and analyzed. Other manipulations were as described as follows, or as is apparent.

Alternately, ADDL formation was carried out as described above, with the exception that the F12 media was replaced by a buffer (i.e., “substitute F12 media”) containing the following components: N,N-dimethylglycine (766 mg/L), D-glucose (1.802 g/L), calcium chloride (33 mg/L), copper sulfate pentahydrate (25 mg/L), iron(II) sulfate heptahydrate (0.8 mg/L), potassium chloride (223 mg/L), magnesium chloride (57 mg/L), sodium chloride (7.6 g/L), sodium bicarbonate (1.18 g/L), disodium hydrogen phosphate (142 mg/L), and zinc sulfate heptahydrate (0.9 mg/L). The pH of the buffer was adjusted to 8.0 using 0.1 M sodium hydroxide.

### Example 2

### Crosslinking of Amyloid β Oligomers

Glutaraldehyde has been successfully used in a variety of biochemical systems. Glutaraldehyde tends to crosslink proteins that are directly in contact, as opposed to nonspecific reaction with high concentrations of monomeric protein. In this example, glutaraldehyde-commanded crosslinking of amyloid β was investigated.

Oligomer preparation was carried out as described in Example 1, with use of substitute F12 media. The supernatant that was obtained following centrifugation (and in some cases, fractionation) was treated with 0.22 mL of a 25% aqueous solution of glutaraldehyde (Aldrich, St. Louis, Mo.), followed by 0.67 mL of 0.175 M sodium borohydride in 0.1 M NaOH (according to the method of Levine, Neurobiology of Aging, 1995). The mixture was stirred at 4° C. for 15 minutes and was quenched by addition of 1.67 mL of 20% aqueous sucrose. The mixture was concentrated 5 fold on a SpeedVac and dialyzed to remove components smaller than 1 kD. The material was analyzed by SDS PAGE. Gel filtration chromatography was carried according to the following: Superose 75PC 3.2/3.0 column (Pharmacia, Upsala, Sweden) was equilibrated with filtered and degassed 0.15% ammonium hydrogen carbonate buffer (pH=7.8) at a flow rate of 0.02 mL/min over the course of 18 h at room temperature. The flow rate was changed to 0.04 mL/min and 20 mL of solvent was eluted. 50 microliters of reaction solution was loaded on to the column and the flow rate was resumed at 0.04 mL/min. Compound elution was monitored via UV detection at 220 nm, and 0.5-1.0 mL fractions were collected during the course of the chromatography. Fraction No. 3, corresponding to the third peak of UV absorbance was isolated and demonstrated by AFM to contain globules 4.9+/−0.8 nm (by width analysis). This fraction was highly neurotoxic when contacted with brain slice neurons, as described in the examples which follow.

### Example 3

### Size Characterization of ADDLs

This example sets forth the size characterization of ADDLs formed as in Example 1 using a variety of methods (e.g., native gel electophoresis, SDS-polyacrylamide gel electrophoresis, AFM, field flow fractionation, immunorecognition, and the like).

AFM was carried out essentially as described previously (e.g., Stine et al. (1996) J. Protein Chem., vol. 15, pp. 193-203). Namely, images were obtained using a Digital Instruments (Santa Barbara, Calif.) Nanoscope 111a Multimode Atomic force microscope using a J-scanner with xy range of 150μ. Tapping Mode was employed for all images using etched silicon TESP Nanoprobes (Digital Instruments). AFM data is analyzed using the Nanoscope IIIa software and the IGOR Pro™ waveform analysis software. For AFM analysis, 4μ scans (i.e., assessment of a 4 μm×4 μm square) were conducted. Dimensions reported herein were obtained by section analysis, and where width analysis was employed, it is specified as being a value obtained by width analysis. Section and width analysis are in separate analysis modules in the Nanoscope 111a software. Generally, for ADDL analysis, there is a systematic deviation between the sizes obtained by section analysis and those obtained by width analysis. Namely, for a 4μ scan, section analysis yields heights that are usually about 0.5 nm taller, thus resulting in a deviation of about 0.5 nm in the values obtained for the sizes of the globules.

Analysis by gel electrophoresis was carried out on 15% polyacrylamide gels and visualized by Coomassie blue staining. ADDLs were resolved on 4-20% tris-glycine gels under non-denaturing conditions (Novex). Electrophoresis was performed at 20 mA for approximately 1.5 hours. Proteins were resolved with SDS-PAGE as described in Zhang et al. (1994) J. Biol. Chem., vol. 269, pp. 25247-25250. Protein was then visualized using silver stain (e.g., as described in Sherchenko et al. (1996) Anal. Chem., vol. 68, pp. 850-858). Gel proteins from both native and SDS gels were transferred to nitrocellulose membranes according to Zhang et al. (J. Biol. Chem., vol. 269, pp. 25247-50 (1994)). Immunoblots were performed with biotinylated 6E10 antibody (Senetak, Inc., St. Louis, Mo.) at 1:5000 and visualized using ECL (Amersham). Typically, gels were scanned using a densitometer. This allowed provision of the computer-generated images of the gels (e.g., versus photographs of the gels themselves).

Size characterization of ADDLs by AFM section analysis (e.g., as described in Stine et al. (1996) J. Protein Chem., vol. 15, pp. 193-203) or width analysis (Nanoscope III software) indicated that the predominant species were globules of about 4.7 nm to about 6.2 nm along the z-axis. Comparison with small globular proteins (Aβ1-40 monomer, aprotinin, bFGF, carbonic anhydrase) suggested that ADDLs had mass between 17-42 kD. What appear to be distinct species can be recognized. These appear to correspond to globules of dimensions of from about 4.9 nm to about 5.4 nm, from about 5.4 nm to about 5.7 nm, and from about 5.7 nm to about 6.2 nm. The globules of dimensions of about 4.9-5.4 nm and 5.7-6.2 nm appear to comprise about 50% of globules.

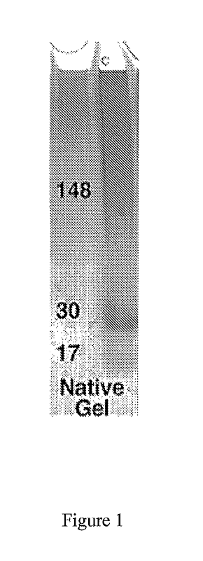

In harmony with the AFM analysis, SDS-PAGE immunoblots of ADDLs identified A□ oligomers of about 17 kD to about 22 kD, with abundant 4 kD monomer present, presumably a breakdown product. Consistent with this interpretation, non-denaturing polyacrylamide gels of ADDLs show scant monomer, with a primary band near 30 kD, a less abundant band at −17 kD, and no evidence of fibrils or aggregates. Computer-generated images of a silver stained native gel and a Coomassie stained SDS-polyacrylamide gel are set out in FIG. 1 and FIG. 2, respectively. The correspondence between the SDS and non-denaturing gels confirms that the small oligomeric size of ADDLs was not due to detergent action. Oligomers seen in ADDL preparations were smaller than clusterin (Mr 80 kD, 40 kD in denatured gels), as expected from use of low clusterin concentrations (1/40 relative to Aβ, which precluded association of Aβ as 1:1 Aβ-clusterin complexes).

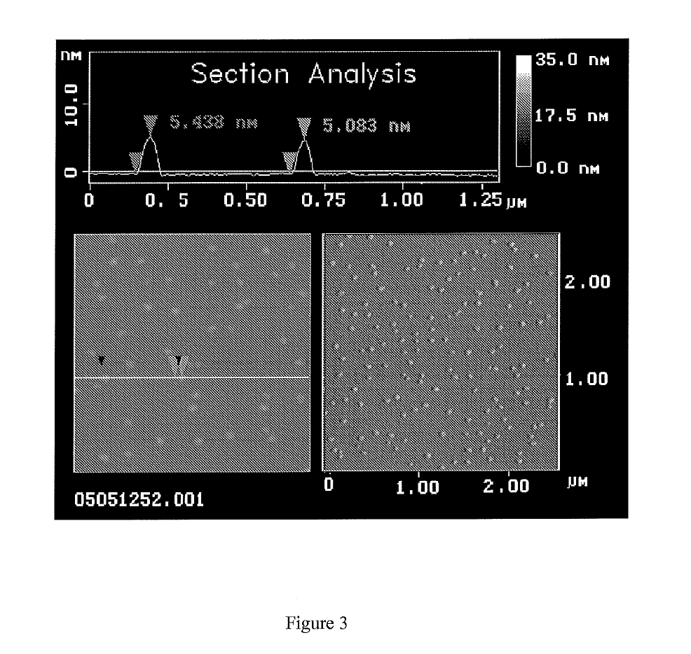

An ADDL preparation according to the invention was fractionated on a Superdex 75 column (Pharmacia, Superose 75PC 3.2/3.0 column). The fraction comprising the ADDLs was the third fraction of UV absorbance eluting from the column and was analyzed by AFM and SDS-polyacryalamide gel electrophoresis. A representative AFM analysis of fraction 3 is depicted in FIG. 3. Fractionation resulted in greater homogeneity for the ADDLs, with the majority of the globules having dimensions of from about 4.9 nm to about 5.4 nm. SDS-polyacrylamide gel electrophoresis of the fraction demonstrated a heavy lower band corresponding to the monomer/dimer form of Aβ. As also observed for the non-fractionated preparation of ADDLs, this appears to be a breakdown product of the ADDLs. Heavier loading of the fraction revealed a larger-size broad band (perhaps a doublet). This further confirms the stability of the non-fibrillar oligomeric Aβ structures to SDS.

### Example 4

### Clusterin Treatment of Amyloid β

Although it has been proposed that fibrillar structures represent the toxic form of Aβ (Lorenzo et al. (1994) Proc. Natl. Acad. Sci. USA, vol. 91, pp. 12243-12247; Howlett et al. (1995) Neurodegen., vol. 4, pp. 23-32), novel neurotoxins that do not behave as sedimentable fibrils will form when Aβ1-42 is incubated with low doses of clusterin, which also is known as “Apo J” (Oda et al. (1995) Exper. Neurol., vol. 136, pp. 22-31; Oda et al. (1994) Biochem. Biophys. Res. Commun., vol. 204, pp. 1131-1136). To test if these slowly sedimenting toxins might still contain small or nascent fibrils, clusterin-treated Aβ preparations were examined by atomic force microscopy.

Clusterin treatment was carried out as described in Oda et al. (Exper. Neurol., vol. 136, pp. 22-31 (1995)) basically by adding clusterin in the incubation described in Example 1. Alternatively, the starting Aβ1-42 could be dissolved in 0.1 N HCl, rather than DMSO, and this starting Aβ1-42 could even have fibrillar structures at the outset. However, incubation with clusterin for 24 hours at room temperature of 37° C. resulted in preparations that were predominantly free of fibrils, consistent with their slow sedimentation. This was confirmed by experiments showing that fibril formation decreases as the amount of clusterin added increases.

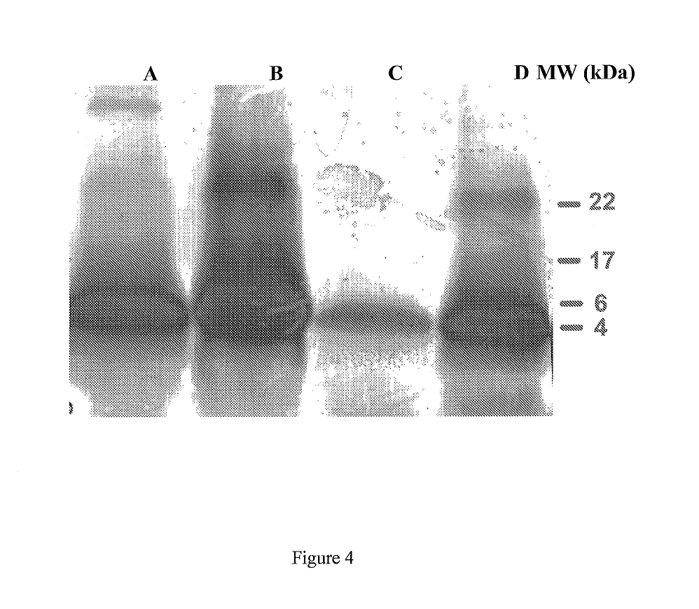

The preparations resulting from clusterin treatment exclusively comprised small globular structures approximately 5-6 nm in size as determined by AFM analysis of ADDLs fractionated on a Superdex 75 gel column. Equivalent results were obtained by conventional electron microscopy. In contrast, Aβ1-42 that had self-associated under standard conditions (Snyder et al. (1994) Biophys. J., vol. 67, pp. 1216-1228) in the absence of clusterin showed primarily large, non-diffusible fibrillar species. Moreover, the resultant ADDL preparations were passed through a Centricon 10 kD cut-off membrane and analyzed on as SDS-polyacrylamide gradient gel. As can be seen in FIG. 4, only the monomer passes through the Centricon 10 filter, whereas ADDLs are retained by the filter. Monomer found after the separation could only be formed from the larger molecular weight species retained by the filter.

These results confirm that toxic ADDL preparations comprise small fibril-free oligomers of Aβ1-42, and that ADDLs can be obtained by appropriate clusterin treatment of amyloid β.

### Example 5

### Physiological Formation of ADDLs

The toxic moieties in Example 4 could comprise rare structures that contain oligomeric Aβ and clusterin. Whereas Oda et al. (Exper. Neurol., vol. 136, pp. 22-31 (1995)) reported that clusterin was found to increase the toxicity of Aβ1-42 solutions, others have found that clusterin at stoichiometric levels protects against Aβ1-40 toxicity (Boggs et al. (1997) J. Neurochem., vol. 67, pp. 1324-1327). Accordingly, ADDL formation in the absence of clusterin further was characterized in this Example.

When monomeric Aβ1-42 solutions were maintained at low temperature in an appropriate media, formation of sedimentable Aβ fibrils was almost completely blocked. A3, however, did self-associate in these low-temperature solutions, forming ADDLs essentially indistinguishable from those chaperoned by clusterin. Finally, ADDLs also formed when monomeric Aβ solutions were incubated at 37 degrees in brain slice culture medium but at very low concentration (50 nM), indicating a potential to form physiologically. All ADDL preparations were relatively stable and showed no conversion to fibrils during the 24 hour tissue culture experiments.

These results confirm that ADDLs form and are stable under physiological conditions and suggest that they similarly can form and are stable in vivo.

### Example 6

### ADDLS are Diffusible, Extremely Potent CNS Neurotoxins

Whether ADDLs were induced by clusterin, low temperature, or low Aβconcentration, the stable oligomers that formed were potent neurotoxins. Toxicity was examined in organotypic mouse brain slice cultures, which provided a physiologically relevant model for mature CNS. Brain tissue was supported at the atmosphere-medium interface by a filter in order to maintain high viability in controls.

For these experiments, brain slices were obtained from mouse strains B6 129 F2 and JR 2385 (Jackson Laboratories, Bar Harbor, Me.) and cultured as previously described (Stoppini et al. (1991) J. Neurosci. Meth., vol. 37, pp. 173-182), with modifications.

Namely, an adult mouse was sacrificed by carbon dioxide inhalation, followed by rapid decapitation. The head was immersed in cold, sterile dissection buffer (94 mL Gey's balanced salt solution, pH 7.2, supplemented with 2 mL 0.5M MgCl₂, 2 ml 25% glucose, and 2 mL 1.0 M Hepes), after which the brain was removed and placed on a sterile Sylgard-coated plate. The cerebellum was removed and a mid-line cut was made to separate the cerebral hemispheres. Each hemisphere was sliced separately. The hemisphere was placed with the mid-line cut down and a 30 degree slice from the dorsal side was made to orient the hemisphere. The hemisphere was glued cut side down on the plastic stage of a Campden tissue chopper (previously wiped with ethanol) and immersed in ice cold sterile buffer. Slices of 200 μm thickness were made from a lateral to medial direction, collecting those in which the hippocampus was visible.

Each slice was transferred with the top end of a sterile pipette to a small petri dish containing Dulbecco's Modified Eagle Medium (DMEM) containing 10% fetal calf serum, 2% S/P/F (streptomycin, penicillin, and fungizone; Life Technologies (Gibco, BRL), Gaithersburg, Md.), observed with a microscope to verify the presence of the hippocampus, and placed on a Millicell-CM insert (Millipore) in a deep well tissue culture dish (Falcon, 6-well dish). Each well contained 1.0 mL of growth medium, and usually two slices were on each insert. Slices were placed in a incubator (6% CO₂, 100% humidity) overnight. Growth medium was removed and wells were washed with 1.0 mL warm Hank-s BSS (Gibco, BRL, Life Technologies). Defined medium (DMEM, N2 supplements, SPF, e.g., as described in Bottenstein et al. (1979) Proc. Natl. Acad. Sci., vol. 76, pp. 514-517) containing the amyloid p oligomers, with or without inhibitor compounds, was added to each well and the incubation was continued for 24 hours.

Cell death was measured using the LIVE/DEAD® assay kit (Molecular Probes, Eugene, Oreg.). This a dual-label fluorescence assay in which live cells are detected by the presence of an esterase that cleaves calcein-AM to calcein, resulting in a green fluorescence. Dead cells take up ethidium homodimer, which intercalates with DNA and has a red fluorescence. The assay was carried out according to the manufacturer's directions at 2 μM ethidium homodimer and 4 μM calcein. Images were obtained within 30 minutes using a Nikon Diaphot microscope equipped with epifluorescence. The MetaMorph image analysis system (Universal Imaging Corporation, Philadelphia, Pa.) was used to quantify the number and/or area of cells showing green or red fluorescence.

For these experiments, ADDLs were present for 24 hours at a maximal 5 μM dose of total Aβ (i.e., total Aβ was never more than 5 μM in any ADDL experiment). Cell death, as shown by “false yellow staining”, was almost completely confined to the stratum pyramidale (CA 3-4) and dentate gyrus (DG) suggesting strongly that principal neurons of the hippocampus (pyramidal and granule cells, respectively) are the targets of ADDL-induced toxicity. Furthermore, glia viability is unaffected by a 24 hour ADDL treatment of primary rat brain glia, as determined by trypan blue exclusion and MTT assay (Finch et al., unpublished). Dentate gyrus (DG) and CA3 regions were particularly sensitive and showed ADDL-evoked cell death in every culture obtained from animals aged P20 (weanlings) to P84 (young adult). Up to 40% of the cells in this region die following chronic exposure to ADDLs. The pattern of neuronal death was not identical to that observed for NMDA, which killed neurons in DG and CA1 but spared CA3.

Some cultures from hippocampal DG and CA3 regions of animals more than 20 days of age were treated with conventional preparations of fibrillar Aβ. Consistent with the non-diffusible nature of the fibrils, no cell death (yellow staining) was evident even at 20 μM. The staining pattern for live cells in this culture verified that the CA3/dentate gyrus region of the hippocampus was being examined. The extent of cell death observed after conventional Aβ treatment (i.e., fibrillar Aβ preparations) was indistinguishable from negative controls in which cultures were given medium, or medium with clusterin supplement. In typical controls, cell death was less than 5%. In fact, high viability in controls could be found even in cultures maintained several days beyond a typical experiment, which confirms that cell survival was not compromised by standard culture conditions.

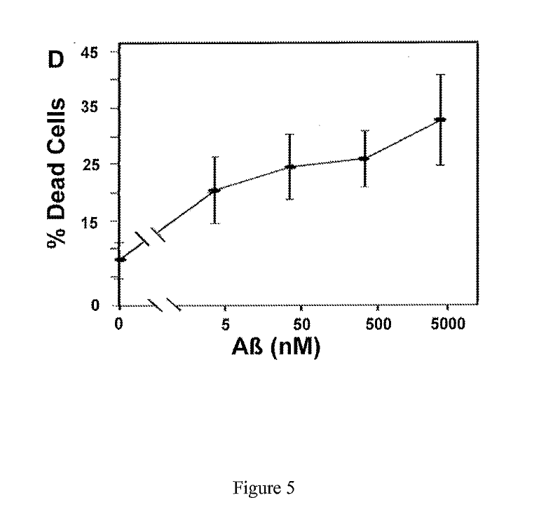

A dose-response experiment was carried out to determine the potency of ADDLs in evoking cell death. Image analysis was used to quantify dead cell and live cell staining in fields containing the DG/CA3 areas. FIG. 5 illustrates the % dead cells verses ADDL concentration measured as initial amyloid β1-42 concentration (nM). Because of the difficulties of quantifying brain slices, the results are not detailed enough to determine the EC50 with precision. However, as can be seen in FIG. 5, even after 1000-fold dilution (˜5 nM Aβ), ADDL-evoked cell death was more than 20%. Toxicity was observed even with 0.3 nM ADDLs. This contrasts with results obtained with conventionally aged Aβ, which is toxic to neurons in culture at about 20 to about 50 μM. These data show that ADDLs are effective at doses approximately 1,000-10,000-fold smaller than those used in fibrillar Aβ experiments.

These data from hippocampal slices thus confirm the ultratoxic nature of ADDLs. Furthermore, because ADDLs had to pass through the culture-support filter to cause cell death, the results validate that ADDLs are diffusible, consistent with their small oligomeric size. Also, the methods set forth herein can be employed as an assay for ADDL-mediated changes in cell viability. In particular, the assay can be carried out by co-incubating or co-administering along with the ADDLs agents that potentially may increase or decrease ADDL formation and/or activity. Results obtained with such co-incubation or co-administration can be compared to results obtained with inclusion of ADDLs alone.

### Example 7

### MTT Oxidative Stress Toxicity Assay—PC12 Cells

This example sets forth an assay that can be employed to detect an early toxicity change in response to amyloid β oligomers.

For these experiments, PC12 cells were passaged at 4×10⁴ cells/well on a 96-well culture plate and grown for 24 hours in DMEM+10% fetal calf serum+1% S/P/F (streptomycin, penicillin, and fungizone). Plates were treated with 200 μg/mL poly-1-lysine for 2 hours prior to cell plating to enhance cell adhesion. One set of six wells was left untreated and fed with fresh media, while another set of wells was treated with the vehicle control (PBS containing 10% 0.01 N HCl, aged o/n at RT). Positive controls were treated with triton (1%) and Na Azide (0.1%) in normal growth media. Amyloid β oligomers prepared as described in Example 1, or obtained upon co-incubation with clusterin, with and without inhibitor compounds present, were added to the cells for 24 hours. After the 24 hour incubation, MTT (0.5 mg/mL) was added to the cells for 2.5 hours (11 μL of 5 mg/ml stock solubilized in PBS into 100 μL of media). Healthy cells reduce the MTT into a formazan blue colored product. After the incubation with MTT, the media was aspirated and 100 μL of 100% DMSO was added to lyse the cells and dissolve the blue crystals. The plate was incubated for 15 min at RT and read on a plate reader (ELISA) at 550 nm.

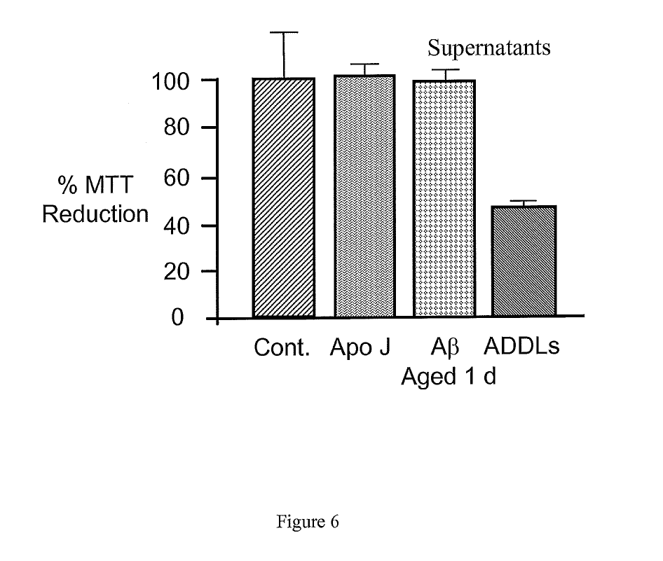

The results of one such experiment are depicted in FIG. 6. As can be seen from this figure, control cells not exposed to ADDLs (“Cont.”), cells exposed to clusterin alone (“Apo J”), and cells exposed to monomeric Aβ (“Aβ”) show no cell toxicity. By contrast, cells exposed to amyloid β co-aggregated with clusterin and aged one day (“Aβ:Apo J”) show a decrease in MTT reduction, evidencing an early toxicity change. The lattermost amyloid preparations were confirmed by AFM to lack amyloid fibrils.

Results of this experiment thus confirm that that ADDL preparations obtained from co-aggregation of Aβ mediated by clusterin have enhanced toxicity. Moreover, the results confirm that the PC 12 oxidative stress response can be employed as an assay to detect early cell changes due to ADDLs. The assay can be carried out by co-incubating or co-administering along with the ADDLs agents that potentially may increase or decrease ADDL formation and/or activity. Results obtained with such co-incubation or co-administration can be compared to results obtained with inclusion of ADDLs alone.

### Example 8

### MTT Oxidative Stress Toxicity Assay—HN2 Cells

This example sets forth a further assay of ADDL-mediated cell changes. Namely, the MTT oxidative stress toxicity assay presented in the preceding example can be carried out with HN2 cells instead of PC 12 cells. Other appropriate cells similarly can be employed.

For this assay, HN2 cells were passaged at 4×10⁴ cells/well on a 96-well culture plate and grown for 24 hours in DMEM+10% fetal calf serum+1% S/P/F (streptomycin, penicillin, and fungizone). Plates were treated with 200 μg/mL poly 1-lysine for 2 hours prior to cell plating to enhance cell adhesion. The cells were differentiated for 24-48 hours with 5 M retinoic acid and growth was further inhibited with 1% serum. One set of wells was left untreated and given fresh media. Another set of wells was treated with the vehicle control (0.2% DMSO). Positive controls were treated with triton (1%) and sodium azide (0.1%). Amyloid P oligomers prepared as described in example 1, with and without inhibitor compounds present, were added to the cells for 24 hours. After the 24 hour incubation, MTT (0.5 mg/mL) was added to the cells for 2.5 hours (11 μL of 5 mg/mL stock into 100 μL of media). After the incubation with MTT, the media was aspirated and 100 μL of 100% DMSO is added to lyse the cells and dissolve the blue crystals. The plate was incubated for minutes at RT and read on a plate reader (ELISA) at 550 nm.

This assay similarly can be carried out by co-incubating or co-administering along with the ADDLs agents that potentially may increase or decrease ADDL formation and/or activity. Results obtained with such co-incubation or co-administration can be compared to results obtained with inclusion of ADDLs alone.

### Example 9

### Cell Morphology by Phase Microscopy

This example sets forth yet another assay of ADDL-mediated cell changes—assay of cell morphology by phase microscopy.

For this assay, cultures were grown to low density (50-60% confluence). To initiate the experiment, the cells were serum-starved in F12 media for 1 hour. Cells were then incubated for 3 hours with amyloid β oligomers prepared as described in example 1, with and without inhibitor compounds added to the cells, for 24 hours. After 3 hours, cells were examined for morphological differences or fixed for immunofluorescence labeling. Samples were examined using the MetaMorph Image Analysis system and an MRI video camera (Universal Imaging, Inc.).

Results of such assays are presented in the examples which follow. In particular, the assay can be carried out by co-incubating or co-administering along with the ADDLs agents that potentially may increase or decrease ADDL formation and/or activity. Results obtained with such co-incubation or co-administration can be compared to results obtained with inclusion of ADDLs alone.

### Example 10

### FACScan Assay for Binding of ADDLs to Cell Surfaces

Because cell surface receptors recently have been identified on glial cells for conventionally prepared Aβ (Yan et al. (1996) Nature, vol. 382, pp. 685-691; El Khouty et al. (1996) Nature, vol. 382, pp. 716-719), and because neuronal death at low ADDL doses suggested possible involvement of signaling mechanisms, experiments were undertaken to determine if specific cell surface binding sites on neurons exist for ADDLs.

For flow cytometry, cells were dissociated with 0.1% trypsin and plated at least overnight onto tissue culture plastic at low density. Cells were removed with cold phosphate buffered saline (PBS)/0.5 mM EDTA, washed three times and resuspended in ice-cold PBS to a final concentration of 500,000 cells/mL. Cells were incubated in cold PBS with amyloid β oligomers prepared as described in Example 1, except that 10% of the amyloid β is an amyloid β1-42 analog containing biocytin at position 1 replacing aspartate. Oligomers with and without inhibitor compounds present were added to the cells for 24 hours. The cells were washed twice in cold PBS to remove free, unbound amyloid β oligomers, resuspended in a 1:1,000 dilution of avidin conjugated to fluorescein, and incubated for one hour at 4° C. with gentle agitation. Alternately, amyloid β-specific antibodies and fluorescent secondary antibody were employed instead of avidin, eliminating the need to incorporate 10% of the biotinylated amyloid β analog. Namely, biotinylated 6E10 monoclonal antibody (1 μL Senetec, Inc., St. Louis, Mo.) was added to the cell suspension and incubated for 30 minutes. Bound antibody was detected after pelleting cells and resuspending in 500 μL PBS, using FITC-conjugated streptavidin (1:500, Jackson Laboratories) for 30 minutes.

Cells were analyzed by a Becton-Dickenson Fluorescence Activated Cell Scanner (FACScan). 10,000 or 20,000 events typically were collected for both forward scatter (size) and fluorescence intensity, and the data were analyzed by Consort 30 software (Becton-Dickinson). Binding was quantified by multiplying mean fluorescence by total number of events, and subtracting value for background cell fluorescence in the presence of 6E10 and FITC.

For these experiments, FACScan analysis was done to compare ADDL immunoreactivity in suspensions of log-phase yeast cells (a largely carbohydrate surface) and of the B103 CNS neuronal cell line (Schubert et al. (1974) Nature, vol. 249, pp. 224-227).

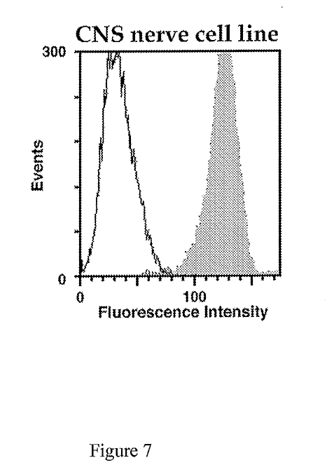

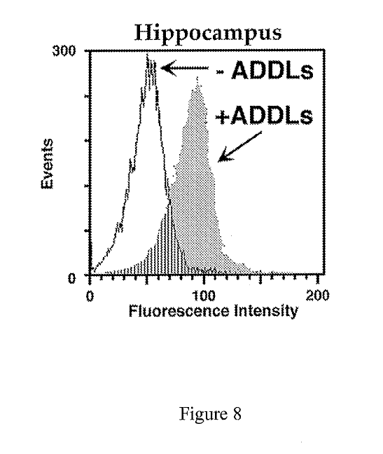

For B103 cells, addition of ADDLs caused a major increase in cell associated fluorescence, as shown in FIG. 7. Trypsin treatment of the B103 cells for 1 minute eliminated ADDL binding. In contrast, control yeast cells (data not shown) demonstrated no ADDL binding, verifying the selectivity of ADDLs for proteins present on the cell surface. Suspensions of hippocampal cells (trypsinized tissue followed by a two-hour metabolic recovery) also bound ADDLs, but with a reduced number of binding events compared with the B103 cells, as evidenced by the reduced fluorescence intensity of the labelled peak. This appears in FIG. 8 as a leftward shifting of the labelled peak.

These results thus suggest that the ADDLs exert their effects by binding to a specific cell surface receptor. In particular, the trypsin sensitivity of B103 cells showed that their ADDL binding sites were cell surface proteins and that binding was selective for a subset of particular domains within these proteins.

Moreover, the present assay can also be employed as an assay for ADDL-mediated cell binding. In particular, the assay can be carried out by co-incubating or co-administering along with the ADDLs agents that potentially may increase or decrease ADDL formation and/or activity. Results obtained with such co-incubation or co-administration can be compared to results obtained with inclusion of ADDLs alone.

### Example 11

### Inhibition of ADDL Formation by Gossypol

This example sets forth the manner in which ADDL formation can be inhibited using, for instance, gossypol.

For these experiments, ADDLs were prepared as described in Example 1. Gossypol (Aldrich) was added to a concentration of 100 μM during the incubation of the Aβ protein to form ADDLs. The resulting preparation was assessed for neurotoxicity using the LIVE/DEAD® assay kit as previously described. The amount of cell death that occurred after 24 hours of exposure to the gossypol/ADDL preparation was less than 5%. This is comparable to the level of toxicity obtained for a corresponding DMSO control preparation (i.e., 6%), or a gossypol control preparation that did not contain any ADDLs (i.e., 4%).

These results thus confirm that compounds such as gossypol can be employed to inhibit ADDL formation.

### Example 12

### Inhibition of ADDL Binding by Tryptic Peptides

Because B103 cell trypsinization was found to block subsequent ADDL binding, experiments were done as set forth in this example to test if tryptic fragments released from the cell surface retard ADDL binding activity.

Tryptic peptides were prepared using confluent B103 cells from four 100 mm dishes. Medium was collected after a 3 minute trypsinization (0.025%, Life Technologies), trypsin-chymotrypsin inhibitor (Sigma, 0.5 mg/mL in Hank's Buffered Saline) was added, and cells were removed via centrifugation at 500×g for 5 minutes. Supernatant (˜12 mL) was concentrated to approximately 1.0 mL using a Centricon 3 filter (Amicon), and was frozen after the protein concentration was determined. For blocking experiments, sterile concentrated tryptic peptides (0.25 mg/mL) were added to the organotypic brain slice or to the suspended B103 cells in the FACs assay at the same time as the ADDLs were added.

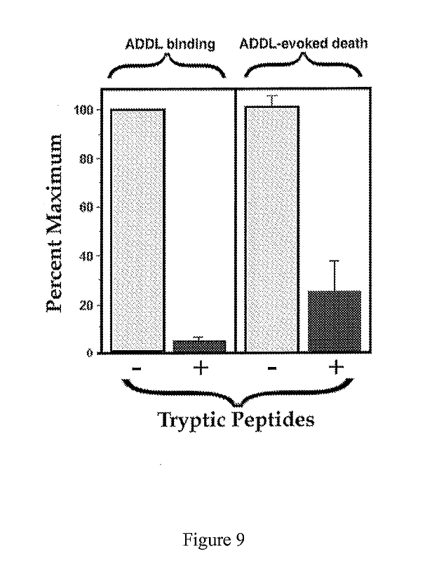

In FACScan assays, tryptic peptides released into the culture media (0.25 mg/mL) inhibited ADDL binding by >90% as shown in FIG. 9. By comparison, control cells exposed to BSA, even at 100 mg/mL, had no loss of binding. Tryptic peptides, if added after ADDLs were already attached to cells, did not significantly lower fluorescence intensities. This indicates that the peptides did not compromise the ability of the assay to quantify bound ADDLs. Besides blocking ADDL binding, the tryptic peptides also were antagonists of ADDL-evoked cell death. Namely, as shown in FIG. 9, addition of tryptic peptides resulted in a 75% reduction in cell death, p<0.002.

These data confirm that particular cell surface proteins mediate ADDL binding, and that solubilized tryptic peptides from the cell surface provide neuroprotective, ADDL-neutralizing activity. Moreover, the present assay can also be employed as an assay for agents that mediate ADDL cell binding or ADDL effects on cell activity. In particular, the assay can be carried out by co-incubating or co-administering along with the ADDLs agents that potentially may increase or decrease ADDL formation and/or activity. Results obtained with such co-incubation or co-administration can be compared to results obtained with inclusion of ADDLs alone. Moreover, addition of the agents before or after binding of the ADDLs to the cell surface can be compared to identify agents that impact such binding, or that act after binding has occurred.

### Example 13

### Dose Response Curve for ADDL Cell Binding

This example sets forth dose response experiments done to determine whether ADDL binding to the cell surface is saturable. Such saturability would be expected if the ADDLs in fact interact with a particular cell surface receptor.

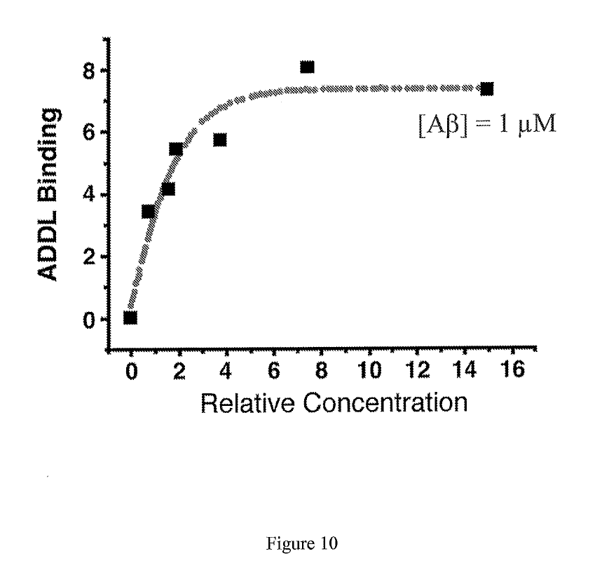

For these studies, B103 cells were incubated with increasing amounts of ADDLs and ADDL binding was quantitated by FACscan analysis. Results are presented in FIG. 10.

These results confirm that a distinct plateau is achieved for ADDL binding. Saturability of ADDL binding occurs at a relative Aβ1-42 concentration (i.e., ADDL concentration relative to Aβ) of about 250 nm.

These results thus confirm that ADDL binding is saturable. Such saturability of ADDL binding, especially when considered with the results of the trypsin studies, validates that the ADDLs are acting through a particular cell surface receptor.

### Example 14

### Cell-Based ELISA for ADDL Binding Activity

This example sets forth a cell-based assay, particularly a cell-based enzyme-linked immunosorbent assay (ELISA) that can be employed to assess ADDL binding activity.

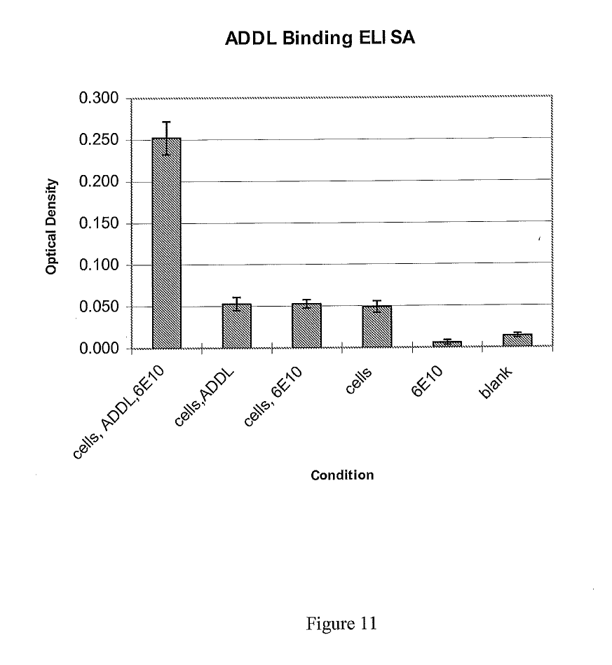

For these studies, 48 hours prior to conduct of the experiment, 2.5×10⁴ B103 cells present as a suspension in 100 μL DMEM were placed in each assay well of a 96-well microtiter plate and kept in an incubator at 37° C. 24 hours prior to the conduct of the experiment, ADDLs were prepared according to the method described in Example 1. To begin the assay, each microtiter plate well containing cells was treated with 50 μL of fixative (3.7% formalin in DMEM) for 10 minutes at room temperature. This fixative/DMEM solution was removed and a second treatment with 50 μL formalin (no DMEM) was carried out for 15 minutes at room temperature. The fixative was removed and each well was washed twice with 100 μL phosphate buffered saline (PBS). 200 μL of a blocking agent (1% BSA in PBS) was added to each well and incubated at room temperature for 1 hour. After 2 washes with 100 μL PBS, 50 μL of ADDLs (previously diluted 1:10 in PBS), were added to the appropriate wells, or PBS alone as a control, and the resulting wells were incubated at 37° C. for 1 hour. 3 washes with 100 μL PBS were carried out, and 50 μL biotinylated 6E10 (Senetek) diluted 1:1000 in 1% BSA/PBS was added to the appropriate wells. In other wells, PBS was added as a control. After incubation for 1 hour at room temperature on a rotator, the wells were washed 3 times with 50 μL PBS, and 50 μL of the ABC reagent (Elite ABC kit, Vector Labs) was added and incubated for 30 minutes at room temperature on the rotator. After washing 4 times with 50 μL PBS, 50 μL of ABTS substrate solution was added to each well and the plate was incubated in the dark at room temperature. The plate was analyzed for increasing absorption at 405 nm. Only when ADDLs, cells, and 6E10 were present was there a significant signal, as illustrated in FIG. 11.

These results further confirm that a cell-based ELISA assay can be employed as an assay for ADDL-mediated cell binding. In particular, the assay can be carried out by co-incubating or co-administering along with the ADDLs agents that potentially may increase or decrease ADDL formation and/or activity. Results obtained with such co-incubation or co-administration can be compared to results obtained with inclusion of ADDLs alone.

### Example 15

### Fyn Kinase Knockout Protects Against ADDL Neurotoxicity

To investigate further the potential involvement of signal transduction in ADDL toxicity, the experiments in this example compared the impact of ADDLs on brain slices from isogenic fyn −/− and fyn +/+ animals. Fyn belongs to the Src-family of protein tyrosine kinases, which are central to multiple cellular signals and responses (Clark, E. A. & Brugge, J. S. (1995) Science, vol. 268, pp. 233-239). Fyn is of particular interest because it is up-regulated in AD-afflicted neurons (Shirazi et al. (1993) Neuroreport, vol. 4, pp. 435-437). It also appears to be activated by conventional Aβ preparations (Zhang et al. (1996) Neurosci. Lett., vol. 211, pp. 187-190) which subsequently have been shown to contain ADDLs by AFM. Fyn knockout mice, moreover, have reduced apoptosis in the developing hippocampus (Grant et al. (1992) Science, vol. 258, pp. 1903-1910).

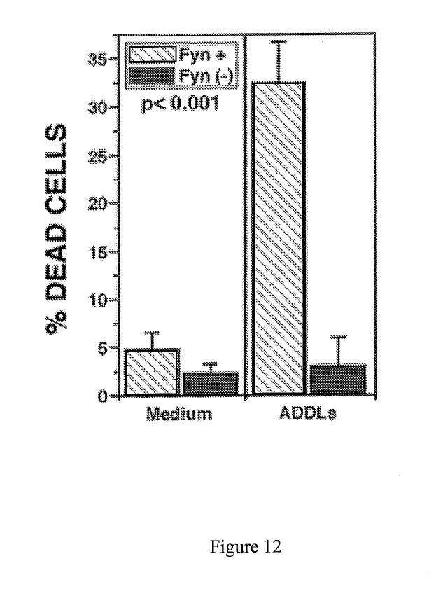

For these studies, Fyn knockout mice (Grant et al. (1992) Science, vol. 258, pp. 1903-1910) were treated as described in the preceding examples, by comparing images of brain slices of mice either treated or not treated with ADDLs for 24 hours to determine dead cells in the DG and CA3 area. The quantitative comparison (presented in FIG. 12) was obtained with error bars representing means+/−SEM for 4-7 slices.

In contrast to cultures from wild-type animals, cultures from fyn −/− animals showed negligible ADDL-evoked cell death, as shown in FIG. 12. For ADDLs, the level of cell death in fyn +/+ slices was more than five times that in fyn −/− cultures. In fyn −/− cultures, cell death in the presence of ADDLs was at background level. The neuroprotective response was selective; hippocampal cell death evoked by NMDA receptor agonists (Bruce et al. (1995) Exper. Neurol., vol. 132, pp. 209-219; Vornov et al. (1991) Neurochem., vol. 56, pp. 996-1006) was unaffected (not shown). Analysis (ANOVA) using the Tukey multiple comparison gave a value of P<0.001 for the ADDL fyn +/+ data compared to all other conditions.

These results confirm that loss of Fyn kinase protected DG and CA3 hippocampal regions from cell death induced by ADDLs. The results validate that ADDL toxicity is mediated by a mechanism blocked by knockout of Fyn protein tyrosine kinase. These results further suggest that neuroprotective benefits can be obtained by treatments that abrogate the activity of Fyn protein tyrosine kinase or the expression of the gene encoding Fyn protein kinase.

### Example 16

### Astrocyte Activation Experiments

To investigate further the potential involvement of signal transduction in ADDL toxicity, the experiments in this example compared the impact on ADDLs on activation of astrocytes.

For these experiments, cortical astrocyte cultures were prepared from neonatal (1-2 day old) Sprague-Dawley rat pups by the method of Levison and McCarthy (Levison et al. (1991) in Culturing Nerve Cells (Banker et al., Eds.), pp. 309-36, MIT Press, Cambridge, Mass.), as previously described (Hu et al. (1996) J. Biol. Chem., vol. 271, pp. 2543-2547). Briefly, cerebral cortex was dissected out, trypsinized, and cells were cultured in α-MEM (Gibco, BRL) containing 10% fetal bovine serum (Hyclone Laboratories Inc., Logan Utah) and antibiotics (100 U/mL penicillin, 100 mg/mL streptomycin). After 11 days in culture, cells were trypsinized and replated into 100-mm plates at a density of ˜6×10⁵ cells/plate and grown until confluent (Hu et al. (1996) J. Biol. Chem., vol. 271, pp. 2543-2547).

Astrocytes were treated with ADDLs prepared according to Example 1, or with Aβ17-42 (synthesized according to Lambert et al. J. Neurosci. Res., vol. 39, pp. 377-384 (1994); also commercially available). Treatment was done by trypsinizing confluent cultures of astrocytes and plating onto 60 mm tissue culture dishes at a density of 1×10⁶ cells/dish (e.g., for RNA analysis and ELISAs), into 4-well chamber slides at 5×10⁴ cells/well (e.g., for immunohistochemistry), or into 96-well plates at a density of 5×10⁴ cells/well (e.g., for NO assays). After 24 hours of incubation, the cells were washed twice with PBS to remove serum, and the cultures incubated in α-MEM containing N2 supplements for an additional 24 hours before addition of Aβ peptides or control buffer (i.e., buffer containing diluent).

Examination of astrocyte morphology was done by examining cells under a Nikon TMS inverted microscope equipped with a Javelin SmartCam camera, Sony video monitor and color video printer. Typically, four arbitrarily selected microscopic fields (20× magnification) were photographed for each experimental condition. Morphological activation was quantified from the photographs with NIH Image by counting the number of activated cells (defined as a cell with one or more processes at least one cell body in length) in the four fields.

The mRNA levels in the cultures was determined with use of Northern blots and slot blots. This was done by exposing cells to ADDLs or control buffer for 24 hours. After this time, the cells were washed twice with diethylpyrocarbonate (DEPC)-treated PBS, and total RNA was isolated by RNeasy purification mini-columns (Qiagen, Inc., Chatsworth, Calif.), as recommended by the manufacturer. Typical yields of RNA were 8 to 30 mg of total RNA per dish. For Northern blot analysis, 5 mg total RNA per sample was separated on an agarose-formaldehyde gel, transferred by capillary action to Hybond-N membrane (Amersham, Arlington Heights Ill.), and UV crosslinked. For slot blot analysis, 200 ng of total RNA per sample was blotted onto Duralon-UV membrane (Stratagene, La Jolla Calif.) under vacuum, and UV crosslinked. Confirmation of equivalent RNA loadings was done by ethidium bromide staining or by hybridization and normalization with a GAPDH probe.

Probes were generated by restriction enzyme digests of plasmids, and subsequent gel purification of the appropriate fragment. Namely, cDNA fragments were prepared by RT-PCR using total RNA from rat cortical astrocytes. RNA was reverse transcribed with a Superscript II system (GIBCO/BRL), and PCR was performed on a PTC-100 thermal controller (MJ Research Inc, Watertown, Mass.) using 35 cycles at the following settings: 52° C. for 40 seconds; 72° C. for 40 seconds; 96° C. for 40 seconds. Primer pairs used to amplify a 447 bp fragment of rat IL-13 were: Forward: 5′ GCACCTTCTTTCCCTTCATC 3′ [SEQ ID NO:1]. Reverse: 5′ TGCTGATGTACCAGTTGGGG 3′ [SEQ ID NO:2]. Primer pairs used to amplify a 435 bp fragment of rat GFAβ were: Forward: 5′ CAGTCCTTGACCTGCGACC 3′ [SEQ ID NO:3]. Reverse: 5′ GCCTCACATCACATCCTTG 3′ [SEQ ID NO:4]. PCR products were cloned into the pCR2.1 vector with the Invitrogen TA cloning kit, and constructs were verified by DNA sequencing. Probes were prepared by EcoRI digestion of the vector, followed by gel purification of the appropriate fragments. The plasmids were the rat iNOS cDNA plasmid pAstNOS-4, corresponding to the rat iNOS cDNA bases 3007-3943 (Galea et al. (1994) J. Neurosci. Res., vol. 37, pp. 406-414), and the rat GAPDH cDNA plasmid pTRI-GAPDH (Ambion, Inc., Austin Tex.).

The probes (25 ng) were labeled with ³²P-dCTP by using a Prime-a-Gene Random-Prime labeling kit (Promega, Madison Wis.) and separated from unincorporated nucleotides by use of push-columns (Stratagene). Hybridization was done under stringent conditions with QuikHyb solution (Stratagene), using the protocol recommended for stringent hybridization. Briefly, prehybridization was conducted at 68° C. for about 30 to 60 minutes, and hybridization was at 68° C. for about 60 minutes. Blots were then washed under stringent conditions and exposed to either autoradiography or phosphoimaging plate. Autoradiograms were scanned with a BioRad GS-670 laser scanner, and band density was quantified with Molecular Analyst v2.1 (BioRad, Hercules Calif.) image analysis software. Phosphoimages were captured on a Storm 840 system (Molecular Dynamics, Sunnyvale Calif.), and band density was quantified with Image Quant v1.1 (Molecular Dynamics) image analysis software.

For measurement of NO by nitrite assay, cells were incubated with Aβ peptides or control buffer for 48 hours, and then nitrite levels in the conditioned media were measured by the Griess reaction as previously described (Hu et al. (1996) J. Biol. Chem., vol. 271, pp. 2543-2547). When the NOS inhibitor N-nitro-L-arginine methylester (L-name) or the inactive D-name isomer were used, these agents were added to the cultures at the same time as the Aβ.

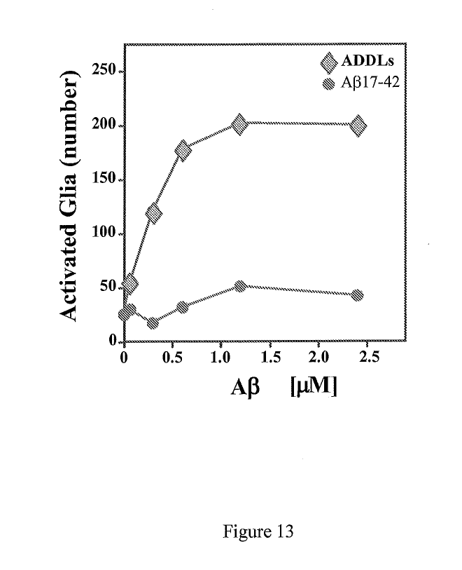

Results of these experiments are presented in FIG. 13. As can be seen in this figure, glia activation increases when astrocytes are incubated with ADDLs, but not when astrocytes are incubated with Aβ17-42.

These results confirm that ADDLs activate glial cells. It is possible that glial proteins may contribute to neural deficits, for instance, as occur in Alzheimer's Disease, and that some effects of ADDLs may actually be mediated indirectly by activation of glial cells. In particular, glial proteins may facilitate formation of ADDLs, or ADDL-mediated effects that occur downstream of receptor binding. Also, it is known that clusterin is upregulated in the brain of the Alzheimer's diseased subject, and clusterin is made at elevated levels only in glial cells that are activated. Based on this, activation of glial cells by a non-ADDL, non-amyloid stimulus could produce clusterin which in turn might lead to ADDLs, which in turn would damage neurons and cause further activation of glial cells.

Regardless of the mechanism, these results further suggest that neuroprotective benefits can be obtained by treatments that modulate (i.e., increase or decrease) ADDL-mediated glial cell activation. Further, the results suggest that blocking these effects on glial cells, apart from blocking the neuronal effects, may be beneficial.

### Example 17

### LTP Assay—ADDLs Disrupt LTP

Long-term potentiation (LTP) is a classic paradigm for synaptic plasticity and a model for memory and learning, faculties that are selectively lost in early stage AD. This example sets forth experiments done to examine the effects of ADDLs on LTP, particularly medial perforant path-granule cell LTP.

Injections of intact animals: Mice were anesthesized with urethane and placed in a sterotaxic apparatus. Body temperature was maintained using a heated water jacket pad. The brain surface was exposed through holes in the skull. Bregma and lambda positions for injection into the middle molecular layer of hippocampus are 2 mm posterior to bregma, 1 mm lateral to the midline, and 1.2-1.5 mm ventral to the brain surface. Amyloid β oligomer injections were by nitrogen puff through ˜10 nm diameter glass pipettes. Volumes of 20-50 nL of amyloid β oligomer solution (180 nM of amyloid β in phosphate buffered saline, PBS) were given over the course of an hour. Control mice received an equivalent volume of PBS alone. The animal was allowed to rest for varying time periods before the LTP stimulus is given (typically 60 minutes).

LTP in injected animals: Experiments follow the paradigm established by Routtenberg and colleagues for LTP in mice (Namgung et al. Brain Research, vol. 689, pp. 85-92 (1995)). Perforant path stimulation from the entorhinal cortex was used, with recording from the middle molecular layer and the cell body of the dentate gyrus. A population excitatory postsynaptic potential (pop-EPSP) and a population spike potential (pop-spike) were observed upon electrical stimulation. LTP could be induced in these responses by a stimulus of 3 trains of 400 Hz, 8×0.4 ms pulses/train (Namgung et al. (1995) Brain Res., vol. 689, pp. 85-92). Recordings were taken for 2-3 hours after the stimulus (i.e., applied at time 0) to determine if LTP is retained. The animal was then sacrificed immediately, or was allowed to recover for either 1, 3, or 7 days and then sacrificed as above. The brain was cryoprotected with 30% sucrose, and then sectioned (30 M) with a microtome. Some sections were placed on slides subbed with gelatin and others were analyzed using a free-floating protocol. Immunohistochemistry was used to monitor changes in GAP-43, in PKC subtypes, and in protein phosphorylation of tau (PHF-1), paxillin, and focal adhesion kinase. Wave forms were analyzed by machine as described previously (Colley et al. (1990) J. Neurosci., vol. 10, pp. 3353-3360). A 2-way ANOVA compares changes in spike amplitude between treated and untreated groups.

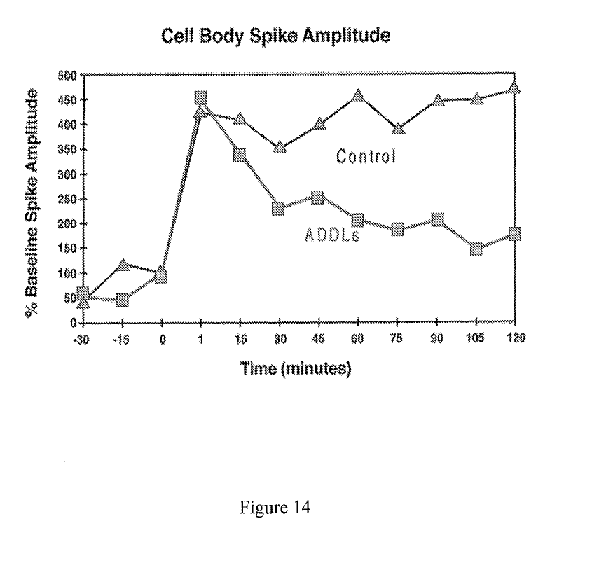

FIG. 14 illustrates the spike amplitude effect of ADDLs in whole animals. As can be clearly seen in this figure, ADDLs block the persistence phase of LTP induced by high frequency electrical stimuli applied to entorhinal cortex and measured as cell body spike amplitude in middle molecular layer of the dentate gyrus.

After the LTP experiment was performed, animals were allowed to recover for various times and then sacrificed using sodium pentobarbitol anesthetic and perfusion with 4% paraformaldehye. For viability studies, times of 3 hours, 24 hours, 3 days, and 7 days were used. The brain was cryoprotected with 30% sucrose and then sectioned (30 μM) with a microtome. Sections were placed on slides subbed with gelatin and stained initially with cresyl violet. Cell loss was measured by counting cell bodies in the dentate gyrus, CA3, CA1, and entorhinal cortex, and correlated with dose and time of exposure of ADDLs. The results of these experiments confirmed that no cell death occurred as of 24 hours following the LTP experiments.

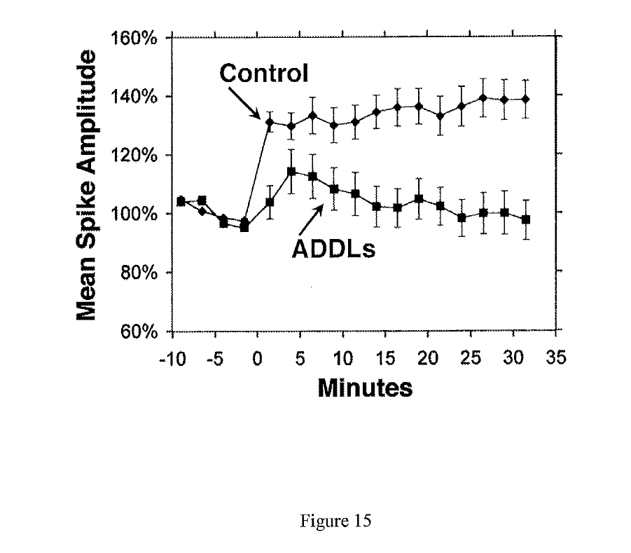

Similarly, the LTP response was examined in hippocampal slices from young adult rats. As can be seen in FIG. 15, incubation of rat hippocampal slices with ADDLs prevents LTP well before any overt signs of cell degeneration. Hippocampal slices (n=6) exposed to 500 nM ADDLs for 45 minutes prior showed no potentiation in the population spike 30 minutes after the tetanic stimulation (mean amplitude 99%+/−7.6), despite a continuing capacity for action potentials. In contrast, LTP was readily induced in slices incubated with vehicle (n=6), with an amplitude of 138%+/−8.1 for the last 10 minutes; this value is comparable to that previously demonstrated in this age group (Trommer et al. (1995) Exper. Neurol., vol. 131, pp. 83-92). Although LTP was absent in ADDL-treated slices, their cells were competent to generate action potentials and showed no signs of degeneration.

These results validate that in both whole animals and tissue slices, the addition of ADDLs results in significant disruption of LTP in less than an hour, prior to any cell degeneration or killing. These experiments thus support that ADDLs exert very early effects, and interference with ADDL formation and/or activity thus can be employed to obtain a therapeutic effect prior to advancement of a disease, disorder, or condition (e.g., Alzheimer's disease) to a stage where cell death results. In other words, these results confirm that decreases in memory occur before neurons die. Interference prior to such cell death thus can be employed to reverse the progression, and potentially restore decreases in memory.

### Example 18

### Early Effects of ADDLs In Vivo

This example sets forth early effects of ADDLs in vivo and the manner in knowledge of such early effects can be manipulated.

The primary symptoms of Alzheimer's disease involve learning and memory deficits. However, the link between behavioral deficits and aggregated amyloid deposits has been difficult to establish. In transgenic mice, overexpressing mutant APP under the control of the platelet-derived growth factor promoter results in the deposition of large amounts of amyloid (Games et al. (1995) Nature, vol. 373, PP. 523-527). By contrast, no behavioral deficits have been reported using this system. Other researchers (i.e., Nalbantoglu, J. et al. (1997) Nature, vol. 387, pp. 500-505; Holcomb, L. et al. (1998) Nat. Med., vol. 4, pp. 97-100) working with transgenic mice report observing significant behavioral and cognitive deficits that occur well before any significant deposits of aggregated amyloid are observed. These behavioral and cognitive defects include failure to long-term potentiate (Nalbantoglu, J. et al., supra). These models collectively suggest that non-deposited forms of amyloid are responsible for the early cognitive and behavioral deficits that occur as a result of induced neuronal malfunction. It is consistent with these models that the novel ADDLs described herein are this non-deposited form of amyloid causing the early cognitive and behavioral defects. In view of this, ADDL modulating compounds according to the invention can be employed in the treatment and/or prevention of these early cognitive and behavioural deficits resulting from ADDL-induced neuronal malfunction, or ADDLs themselves can be applied, for instance, in animal models, to study such induced neuronal malfunction.

Similarly, in elderly humans, cognitive decline and focal memory deficits can occur well before a diagnosis of probable stage I Alzheimer's disease is made (Linn et al. (1995) Arch. Neurol., vol. 52, pp. 485-490). These focal memory deficits may result from induced abberant signaling in neurons, rather than cell death. Other functions, such as higher order writing skills (Snowdon et al. (1996) JAMA, vol. 275, pp. 528-532) also may be affected by abberant neuronal function that occurs long before cell death. It is consistent with what is known regarding these defects, and the information regarding ADDLs provided herein, that ADDLs induce these defects in a manner similar to compromised LTP function such as is induced by ADDLs. Along these lines, ADDL modulating compounds according to the invention can be employed in the treatment and/or prevention of these early cognitive decline and focal memory deficits, and impairment of higher order writing skills, resulting from ADDL formation or activity, or ADDLs themselves can be applied, for instance, in animal models, to study such induced defects. In particular, such studies can be conducted such as is known to those skilled in the art, for instance by comparing treated or placebo-treated age-matched subjects.

### Example 19

### Further Method for Preparing Amyloid β Oligomers (ADDLs)

This Example describes an alternative method for making ADDLs that can be employed instead of, for instance, the methods described in Examples 1 and 4.

Amyloid β monomer stock stock solution is made by dissolving the monomer in hexafluoroisoproanol (HFIP), which is subsequently removed by speed vacuum evaporation. The solid peptide is redissolved in dry DMSO at 5 mM to form a DMSO stock solution, and the ADDLs are prepared by diluting 1 μl of the DMSO stock solution into 49 μl of F12 media (serum-free, phenol-red free). The mixture is vortexed and then incubated at 4° C. for 24 hours.

### Example 20

### Further Gel Studies of Amyloid/Oligomers

This Example describes further gel studies done on amyloid β oligomers.

For gel analysis following preparation of the amyloid β oligomers (i.e., oligomers prepared as described in the prior example), 1 μl of the oligomer solution is added to 4 μl of F12 and 5 μl of tris-tricine loading buffer, and then loaded on a pre-made 16.5% tris-tricine gel (Biorad). Electrophoresis is carried out for 2.25 hours at 100 V. Following electrophoresis, the gel is stained using the Silver Xpress kit (Novex). Alternately, instead of staining the gel, the amyloid β species are transferred from the gel to Hybond-ECL (Amersham) in SDS-containing transfer buffer for 1 hour at 100 V at 4° C. The blot is blocked in TBS-T1 containing 5% milk for 1 hour at room temperature. Following washing in TBS-T1, the blot is incubated with primary antibody (26D6, 1:2000) for 1.5 hours at room temperature. The 26D6 antibody recognizes the amino terminal region of amyloid 3. Following further washing, the blot is incubated with secondary antibody (anti-mouse HRP, 1:3500) for 1.5 hours at room temperature. Following more washing, the blot is incubated in West Pico Supersignal reagents (500 μl of each, supplied by Pierce) and 3 mls of ddH₂O for minutes. Finally, the blot is exposed to film and developed.

Results of such further gel studies are depicted in FIG. 16, which shows a computer-generated image of a densitometer-scanned 16.5% tris-tricine SDS-polyacrylamide gel (Biorad). The figure confirms a range of oligomeric, soluble ADDLs (labeled “ADDLs”), dimer (labeled “Dimer”), and monomer (labeled “Monomer”). This gel system thus enables visualization of distinct ADDLs comprising from at least 3 monomers (trimer) up to about 24 monomers.

What is not depicted in FIG. 16, but which becomes apparent upon comparing gels/Westerns obtained before and after aggregation is the fact that the tetramer band increases upon aggregation, whereas the pentamer through the 24-mer oligomer species appear only after aggregation. The differences between the silver stained and the immunodetected amounts of the oligomers (especially dimer and tetramer) suggest that the oligomers may represent different conformations obtained upon aggregation.

### Example 21

### Further AFM Studies of Amyloid β Oligomers

This Example describes further AFM studies done on amyloid β oligomers.

AFM was done as described in Example 3 except that fractionation on a Superdex 75 column was not performed, and the field was specifically selected such that larger size globules in the field were measured. The analysis is the same from a technical standpoint as that done in Example 3, but in this instance the field that was specifically selected for and examined allows visualization of oligomers that have larger sizes than those that were measured by the section analysis. AFM was carried out using a NanoScope® III MultiMode AFM (MMAFM) workstation using TappingMode® (Digital Instruments, Santa Barbara, Calif.).

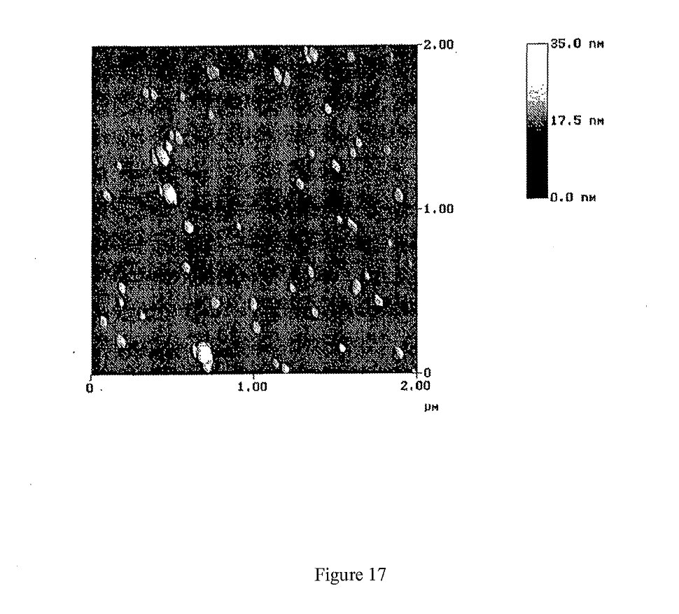

The results of these studies are shown in FIG. 17, which is a computer-generated image of an AFM analysis of ADDLs showing various sized structures of different amyloid β oligomers. The adhered structures range in size from 1 to 10.5 nm in z height. Based on this characterization, the structures comprise from 3 to 24 monomeric subunits, consistent with the bands shown on Tris-tricine SDS-PAGE. In separate experiments (not shown) species as high as about 11 nm have been observed.

### Example 22

### Preparation, Characterization and Use of Anti-ADDL Antibodies

### Materials & Methods

Materials: Aβ₁₋₄₂ was obtained from American Peptide. Cell culture products were obtained from CellGro and Life Technologies. Unless otherwise indicated, chemicals and reagents were from Sigma-Aldrich. The following kits were used: the Boehringer Mannheim Cell Proliferation (MTT) kit, the Novex Silver Xpress kit, and the Pierce West Femto kit for chemiluminescence. SDS-PAGE gels and buffers were from BioRad. Antibodies 6E10, 6E100Bi, and 4G8 were obtained from Senetek. 26D6 was a gift of Sibia Corporation. Conjugated secondary antibodies were obtained from Jackson Labs and Amersham.

Aβ derived diffusible ligand (ADDL) preparation: Aβ₁₋₄₂ was dissolved in hexafluoro-2-propanol (HFIP) and aliquoted to microcentifuge tubes. HFIP was removed by lyophylization and the tubes were stored at −20° C. An aliquot of Aβ₁₋₄₂ was dissolved in anhydrous DMSO to make a 5 mM solution. The DMSO solution was then added to cold F12 medium (Life Technologies) to make a 100 M solution. This solution was incubated at 4° C. for at least 24 hours and then centrifuged at 14,000×g for 10 min. The supernatant is ADDLs, used usually at a 1:10 or 1:20 dilution in medium.

MTT assay: PC12 cells were plated at 30,000 cells/well in 96-well plates and grown overnight. This medium was removed and ADDLs (5 or 10 M) or vehicle were added in new medium (F12K, 1% horse serum, antibiotic/antimycotic). After 4 hrs at 37° C., MTT (10 μl) was added to each well and allowed to incubate for 4 hours at 37° C. The solubilization buffer (100 μl) was added and the plate was placed at 37° C. overnight. The assay was quantified by reading at 550 or 550/690 nm on a plate reader; data were plotted as averages with standard error of the mean (SEM).

Silver stain: The procedure outlined by the manufacturer (Novex) was followed.

Antibody preparation: The polyclonal antibodies were produced and purified by Bethyl Laboratories, Inc., Texas. The initial 24-hour material was sent overnight on ice to the antibody company. It was diluted with complete Freund's adjuvant at 1:1 and injected the day it was received. Antigen labeled +48 hours was thus the material injected. Booster injections continued over several weeks and used incomplete adjuvant. Hyperimmune serum produced in two rabbits was quantified by ELISA against the original antigen solution in a 96-well format. After attainment of an appropriate antibody titer, the animals were bled and antibodies were then collected and purified using an affinity column. The affinity column was prepared by linking an Aβ40 solution (50 μg/ml gel) to agarose via a cyanogen bromide method. Binding of the appropriate antibodies to the column was monitored by ELISA. The polyclonal antibodies were then removed from the column, fractionated using ammonium sulfate precipitation and ion-exchange chromatography, and sent to us as an IgG preparation of >95% purity. We received antibodies from two rabbits (M93 and M94) which were each bled a total of three times.

Immunoblotting: Previously published procedures were followed (Zhang, C. et al. (1994) J. Biol. Chem., vol. 269, pp. 25247-25250). Briefly, equal amounts of protein or ADDLs were added to sample buffer and loaded on a 16.5% Tris-Tricine gel. For mixed samples, ADDLs were added to protein just before sample buffer and then placed immediately on the gel. The proteins were separated by electrophoresis at 100 v until the sample buffer reached the bottom of the gel. Proteins were then transferred to nitrocellulose at 100 v for 1 hr in the cold. The membrane was blocked for 1 hr at RT with 5% non-fat dry milk in Tris-buffered saline with 0.1% triton. The sample was incubated with primary antibody for 1.5 hr at RT and washed 3×15 min. Primary antibody was usually used at a dilution of 1:2000, equivalent to a protein concentration between 0.3 and 0.6 μg/ml, depending on the antibody used. The membrane was incubated with secondary antibody for 1 hr at RT (usually a dilution of 1:20,000) and washed the same way. Proteins were visualized with chemiluminescence. Quantification utilized Kodak 1D Image Analysis software for the IS440CF Image Station.

Preparation of rat hippocampal cultures: The procedure of Brewer (Brewer, G. J. (1997) J. Neurosci., vol. 71, pp. 143-155) for preparation of embryonic mouse cultures was followed. The hippocampus was removed from the animal and placed in Hibernate™/B27 medium until all hippocampii were dissected and cleaned. The tissue was then dissociated with papain. Cells were separated by trituration, recombined, and plated on glass coverslips coated with poly-L-lysine (200 μg/ml) and laminin (15 μg/ml). Plating medium was Neurobasal™-E/B27, supplemented with 0.5 mM glutamine, 5 ng/ml β-FGF, and antibiotic/antimycotic (Life Technologies). This procedure usually gave us clean, primarily neuronal, cultures and cells that developed long processes. If cultures were not used by three days, the medium was replaced with fresh medium.

ADDL immunofluorescence: Cells were cultured on coated glass coverslips as described previously (Stevens, G. R. et al. (1996) J. Neurosci. Res., vol. 46, pp. 445-455). ADDLs were added to cells in serum-free medium for varied times. Free ADDLs were removed by washing with warm medium. Cells were fixed at room temperature in 1.88% formaldehyde for 10 minutes, followed by a post-fix for 15 min. in 3.7% formaldehyde. Bound ADDLs were identified by incubation with M94 polyclonal antibody and visualized using anti-rabbit IgG conjugated to Oregon Green-514 (Jackson Labs). A Nikon Diaphot inverted microscope equipped for epifluorescence was used for analysis.

### Results

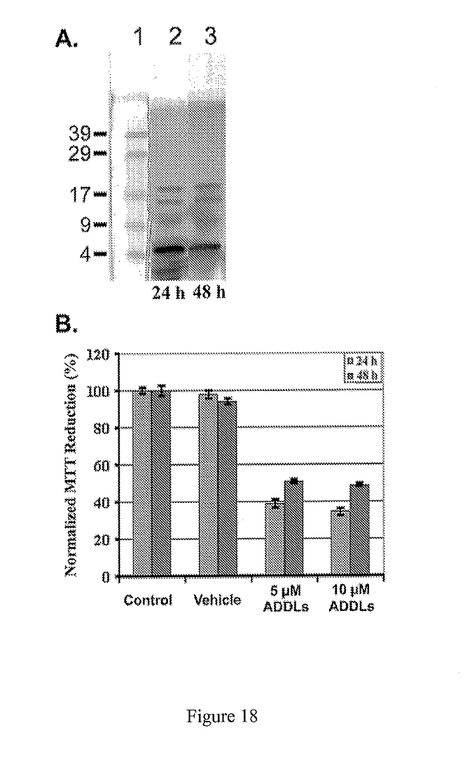

In order to immunize with defined ADDL antigens, we first verified that our preparations consistently provided expected structure and neurotoxicity. ADDL solutions should contain only monomer and toxic oligomers (Lambert, M. P. et al. (1998) Proc. Natl. Acad. Sci. USA, vol. 95, pp. 6448-6453). To eliminate seeds that promote fibril formation, Aβ1-42 from the supplier was first monomerized by dissolving in hexafluoro-isopropanol (HFIP) and then dried for storage (Stine, W. B. et al. (2000) Soc. Neurosci. Abstr., vol. 26, p. 800). This monomerized Aβ₁₋₄₂ was used weekly for 8 weeks, reliably giving ADDLs that were at the same concentration (0.24±0.01 mg Aβ/ml; see Methods). Atomic force microscopy verified that ADDL solutions were fibril-free (not shown), confirming previous observations (Lambert, M. P. et al. (1998) Proc. Natl. Acad. Sci. USA, vol. 95, pp. 6448-6453). Constituents of each preparation were analyzed further by SDS-PAGE and silver staining and found to consist exclusively of small oligomers and monomers (the predominant constituent, 45±5%). FIG. 18A illustrates the composition of a preparation used for immunization. The time points show the status of the initial preparation and the same preparation one day later. There was no change in composition with time. Each preparation also was tested for toxicity to PC12 cells as assayed by impact on MTT reduction. Whether measured immediately after preparation, or one day later, the ADDL solutions showed consistent potency in blocking MTT reduction (FIG. 1B). Impact was essentially maximal by 5 μM. These results established that immunogens were consistent throughout the course of the study with respect to protein concentration, oligomer profile, and toxic activity.

ADDL solutions prepared as above (0.23 mg/ml total protein, see Methods) were mixed with 1 ml complete Freund's adjuvant and injected immediately into two rabbits (0.12 mg protein/animal). Booster injections (5) used incomplete adjuvant and continued over 10 weeks. The rabbits were bled three times to obtain antsera (M93 and M94) which were purified by affinity chromatography and fractionated giving an IgG preparation>95% pure.

The ability of the new antibodies to identify various Aβ species was assessed by immunoblots. Results were compared with those of standard monoclonal antibodies 4G8, 26D6, and 6E10. 26D6 (Kounnas, M. Z., personal communication) and 6E10 (Kim, K. S. et al. (1990) Neurosci. Res. Commun., vol. 7, pp. 113-122) recognize similar epitopes of Aβ, aa1-12 and 1-16, respectively; 4G8 recognizes aa17-24 of Aβ (Enya, M. et at. (1999) Am. J. Pathol., vol. 154, pp. 271-279). Comparisons showed similar efficacies but marked differences in specificity. The three monoclonals recognize monomers as well as oligomeric species. 4G8 also is particularly effective at binding small amounts of dimer. In contrast, the new polyclonal antibodies showed strong preference for oligomeric species. Applied to the same preparation of ADDLs, and in a dose equal to the monoclonals, M94 and M93 recognized only trimer and tetramer (FIGS. 19 and 20). Dose response data showed that M93 can bind monomer but only at high concentrations of antibody (FIG. 20). At a dilution at which 6E10 will bind monomer at least as well as oligomers, the M93 antibodies bind only oligomers. Dimer is not recognized by either antibody. These data indicate that the polyclonal antibodies readily recognize higher organized forms of Aβ, but not monomer.

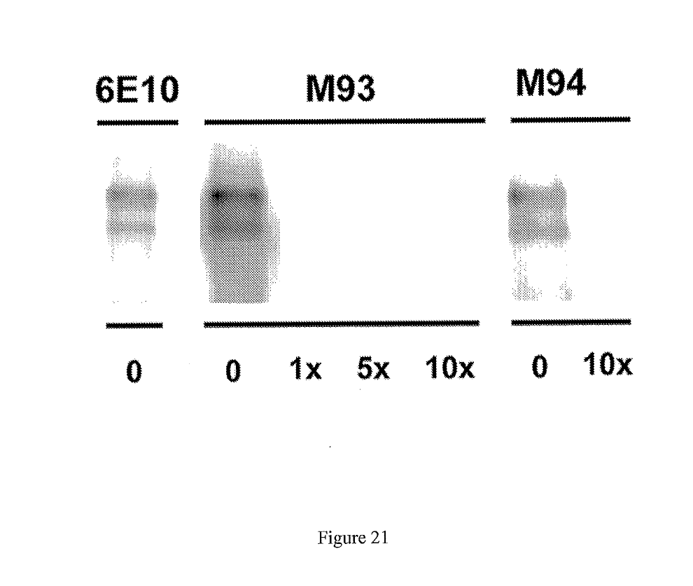

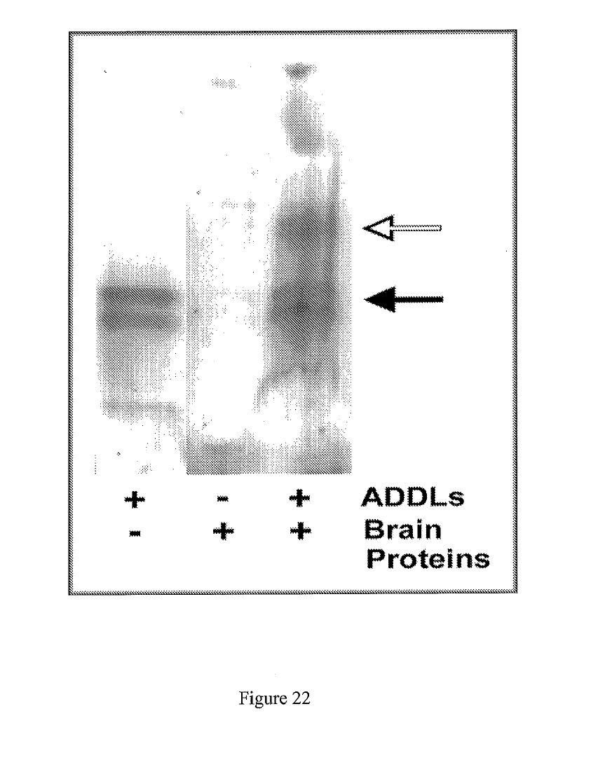

Possible non-specific association of antibodies with ADDLs was tested by pre-absorbing antibodies with ADDLs for 2 hours at 4° C. Pre-absorption eliminated all binding in the immunoblot (FIG. 21). To determine if the antibodies might bind non-specifically to neural proteins other than ADDLs, immunoblots were carried out using homogenates from rat brain. The results show little reaction with any proteins in the homogenate (FIG. 22, middle lane). Similar results were obtained with rat postmitochondrial membrane homogenates and B103 CNS neuroblastoma cell homogenates (not shown). To test if the antibodies can detect ADDLs in the presence of other brain proteins, ADDLs were added to the homogenate before the gel separation and then immunoblotted (FIG. 22, right lane). Trimer and tetramer (filled arrow) were detected, and in addition, the antibodies recognized higher molecular weight species. The most prominent of these bands are indicated by the open arrow, with trace amounts showing up at higher molecular weights. The higher molecular weight species may be larger oligomers, as previously found in human brain (Guerette, P. A. et al. (2000) Soc. Neurosci. Abstr., vol. 25, p. 2129), or perhaps a complex between ADDLs and a second protein such as ApoE (LaDu, M. J. et al. (1995) J. Biol. Chem., vol. 270, pp. 9039-9042).

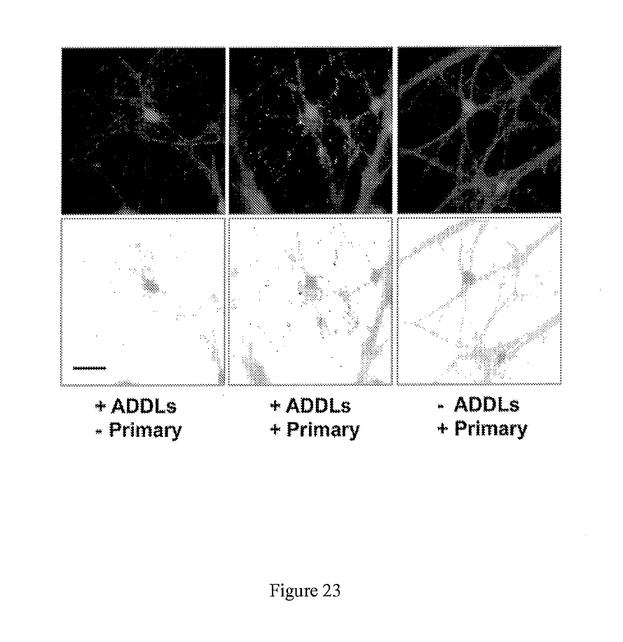

Since the antibodies recognized ADDLs in the presence of other brain proteins, we next tested if they might be useful for microscopy to detect ADDLs bound to cells in culture. Cultures were prepared from E18 rat hippocampus and incubated with ADDLs for 90 min. at 37° C. (see Methods). Cells were fixed, incubated with M94, and visualized with a secondary IgG conjugated to Oregon green-514. No signal was seen without ADDLs, consistent with the specificity found in immunoblots. In the presence of ADDLs, M94 detected small puncta localized almost exclusively to neurites (FIG. 23). This punctate binding is similar to that found when ADDLs are visualized with commercially available antibodies (Viola, K. L. et al. (2000) Soc. Neurosci. Abstr., vol. 26, p. 1285).

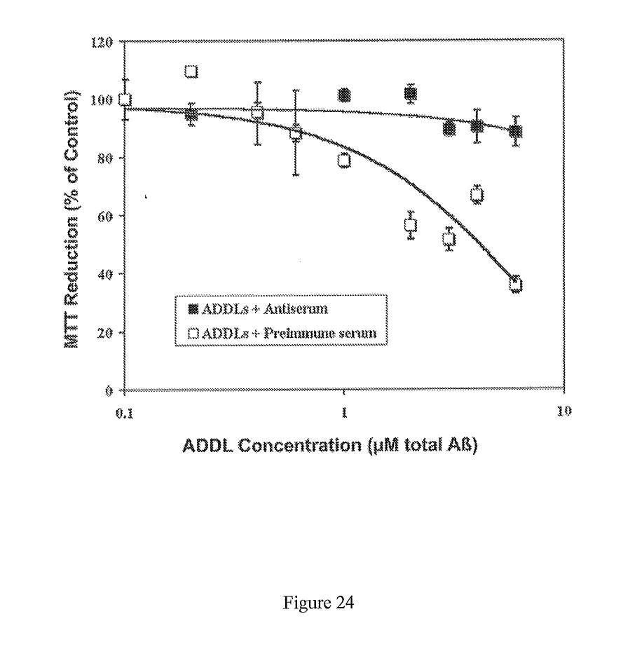

The final experiment was designed to test if the antibodies might target ADDLs in solution and prevent their neurotoxicity. Toxicity was assessed by the impact of ADDLs on MTT reduction in PC12 cells (Shearman, M. S. et al. (1994) Proc. Natl. Acad. Sci. USA, vol. 91, pp. 1470-1474; Liu, Y. et al. (1998) Proc. Natl. Acad. Sci. USA, vol. 95, pp. 13266-13271; Liu, Y. & Schubert, D. (1997) J. Neurochem., vol. 69, pp. 2285-2293; Oda, T. et al. (1995) Exp. Neurol., vol. 136, pp. 22-31; Lambert, M. P. et al. (2000) Soc. Neurosci. Abstr., vol. 26, p. 1285. Control assays of ADDL activity in the presence of pre-immune serum showed a dose-dependent blockade of MTT reduction (FIG. 24, open squares). To test for possible protection, antibodies and ADDLs were incubated together for 2 hours before being assayed. In this case, ADDLs were no longer active (FIG. 24, filled squares). Data shown are for a 4-hour impact of ADDLs. Equivalent results were obtained in tests of a 24-hour impact (not shown). In addition, protection occurred whether ADDLs were made with the chaperone clusterin or under chaperone-free conditions (not shown). These results demonstrate a potent ability of ADDL antibodies to neutralize neurotoxicity.

### Example 23

### ADDLs as a Serum-Based Biomarker for Alzheimer's Disease and Mild Cognitive Impairment (MCI)

As discussed above, Alzheimer's disease and mild cognitive impairment can be caused by ADDLs. It is well known in the art that cognitive function can be quantitatively measured by numerous methods. As described above, ADDLs can be quantitatively measured in serum, and post mortem, in the brain. Thus, it is possible to establish a statistical correlation between cognitive function just prior to death, with ADDL concentration in the brain post-mortem. Furthermore, establishing a statistical correlation between brain ADDLs and serum ADDLs allows for a clinical diagnosis of Alzheimer's disease and MCI while the subject or patient is in the early stages of the disease.

Therefore, ADDLs can be utilized as a biomarker for these diseases, in a manner very similar to using serum cholesterol measurements as a biomarker for coronary heart disease. Currently, there are no such serum-based markers for AD or MCI.

The utility of establishing ADDLs as a biomarker of AD and MCI includes, but is not limited to:
a. such a biomarker can be used to enable monoclonal antibody-based serum diagnostic assays;
b. such a biomarker can be used to assist in the qualification of patients for clinical trials, improving signal-to-noise compared to current clinical protocols that lack this screening biomarker, thereby making such tests shorter and/or smaller in size resulting in considerable cost savings;
c. such a biomarker can be used to provide early diagnosis and rate of disease progression over time;
d. such a biomarker can be used to determine the effectiveness of therapeutic and/or prophylactic pharmaceutical interventions;
e. such a biomarker can be used to determine the effectiveness of DHEA-regulated ingredients (i.e., nutraceuticals and the like) in reducing the levels of ADDLs in serum, brain, and cerebro-spinal fluid (CSF).

### Example 24

### ADDL Binding Proteins

### Characteristics of ADDLs and ADDL Receptors in Human Brain

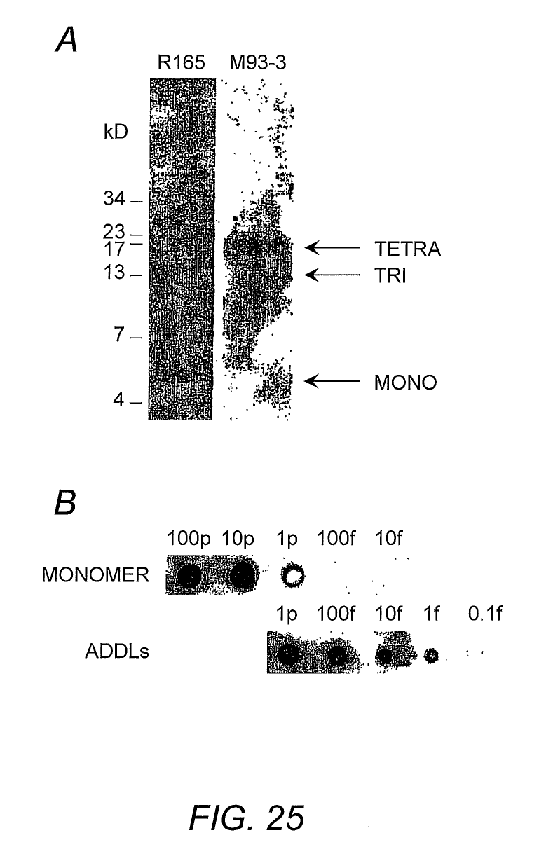

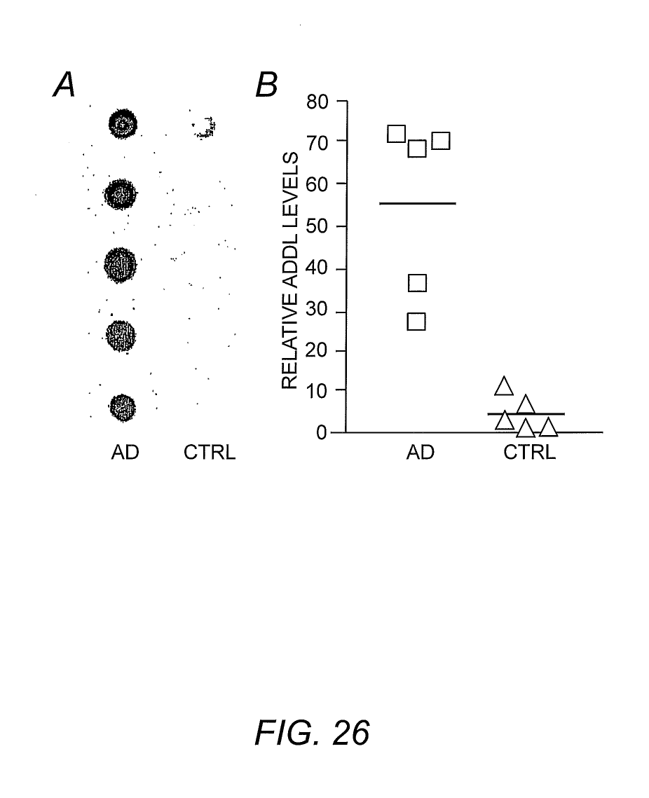

As shown in FIGS. 25 and 26, highly sensitive assays for ADDLs enable the detection of ADDLs in AD brain. ADDLs are elevated by as much as 70 fold in AD brain, compared with non-AD brain.

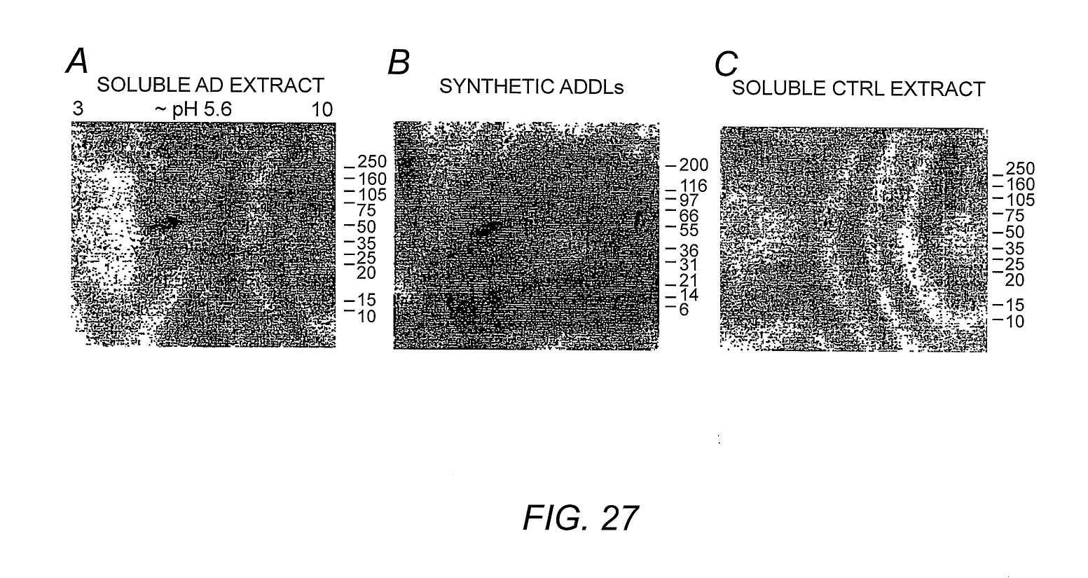

Furthermore, as shown in FIG. 27, ADDLs in human brain are identical to larger oligomers present in ADDL samples prepared from synthetic Aβ1-42.

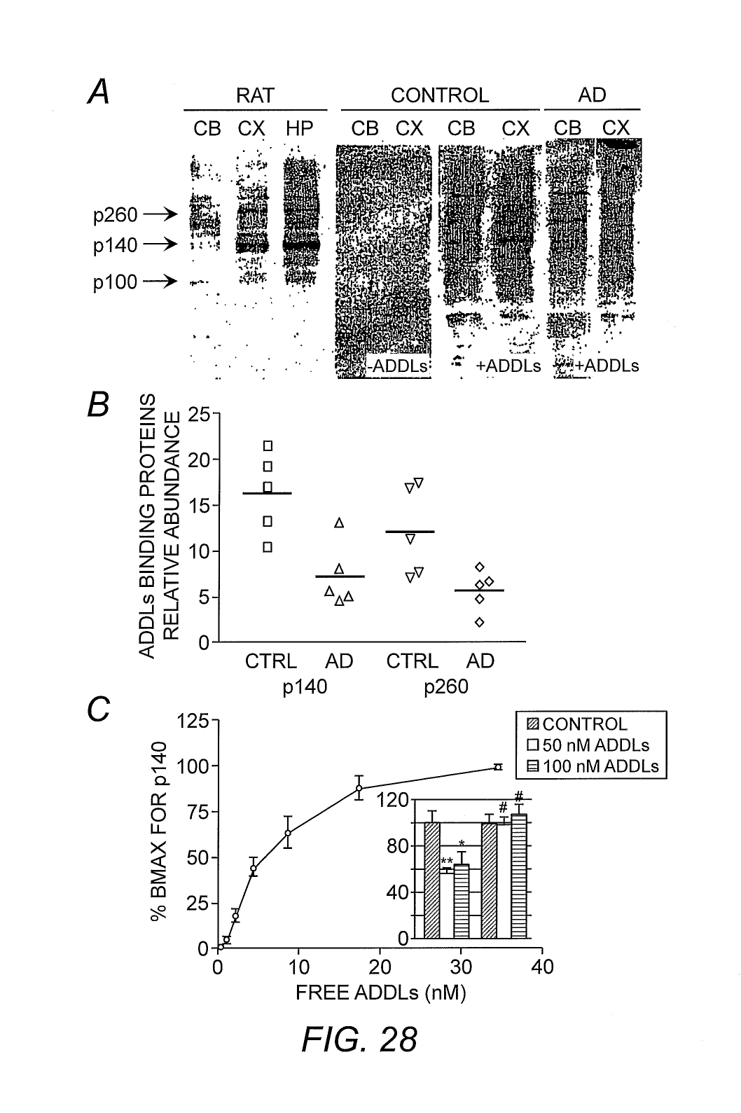

ADDLs bind to 3 protein bands isolated from nerve cell membranes from cortex and hippocampus, but not from cerebellum. The receptor proteins are found in rat brain and human brain, and the bands are depleted from the cortex of AD patients. (see FIG. 28)

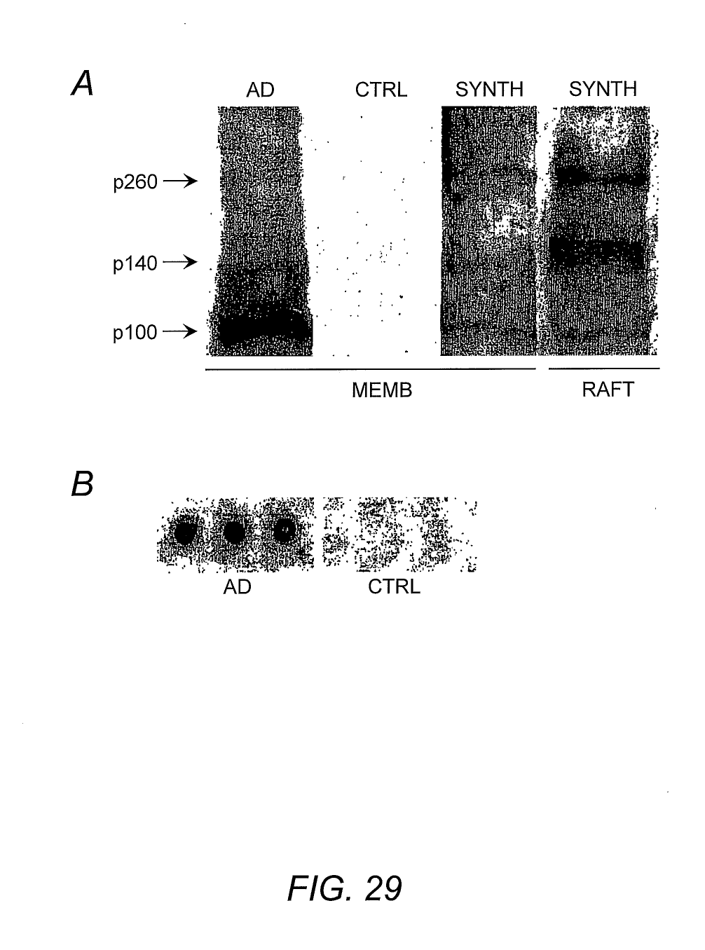

ADDLs isolated from human brain or prepared from synthetic Aβ1-42 exhibit specific binding to proteins with molecular weights (MWs) of approximately 100 kDa, approximately 140 kDa, and approximately 260 kDa. (see FIG. 29)

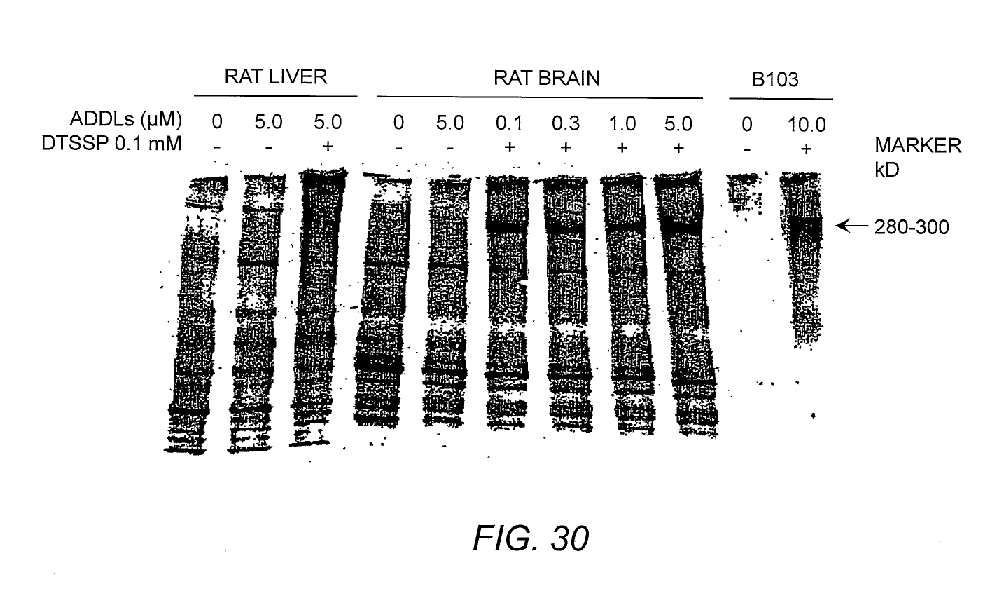

As shown in FIG. 30, ADDLs bind to p260 from B103 cells or brain tissue and crosslinking generates ADDL-p260 complex with a MW ranging from 280-300 kDa. In order to identify specific ADDL binding proteins on nerve cell surfaces, a crosslinking reagent was added after incubation of ADDLs with membrane proteins isolated from brain tissue or B103 neurons. The proteins were then separated by gel electrophoresis, blotted and probed with an ADDL-specific antibody (M94-3). One ADDL-dependent band with a molecular weight (MW) of about 250-300 kDa was found in B103 and rat brain membranes, but not in liver membranes.

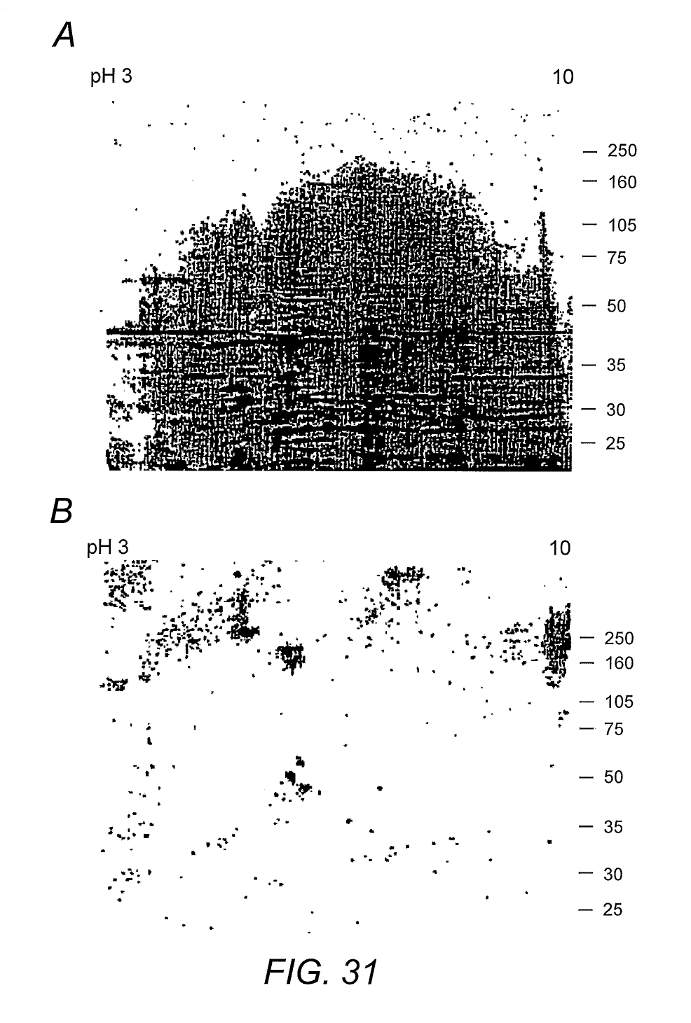

The putative ADDL receptor p260 is a non-abundant protein with a pI of about 5.6. (see FIG. 31)

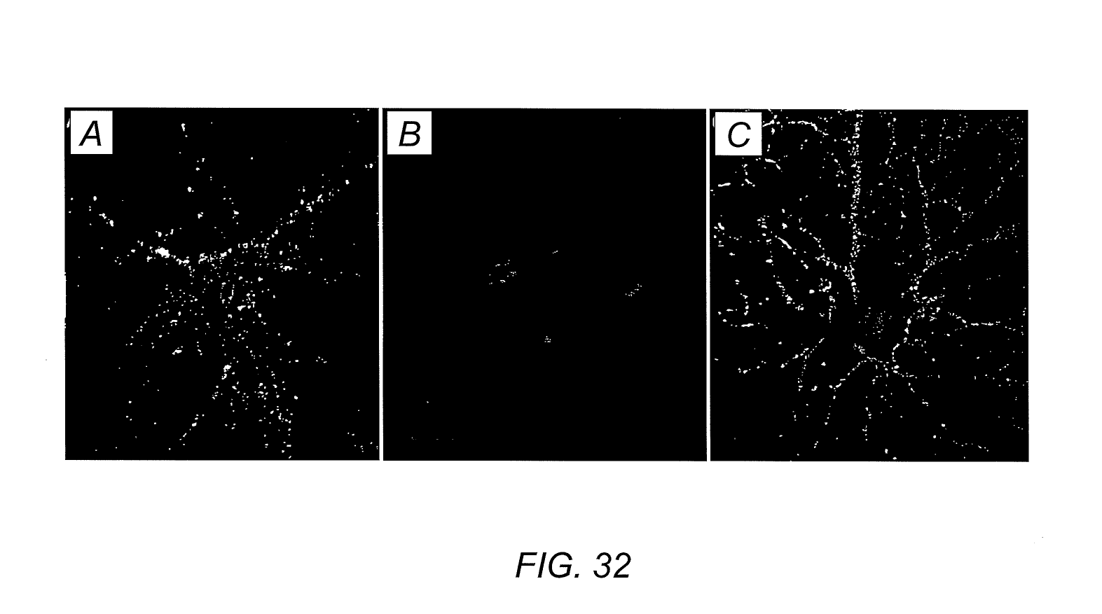

As shown in FIG. 32, ADDLs bind to receptors to form distinct “puncta”, predominantly on processes, but also on the cell body of nerve cells. Treatment of hippocampal brain slices with ADDLs results in rapid blockage of LTP (Lambert M. P. et al. (1998) Proc. Natl. Acad. Sci. USA, vol. 95, no. 11, pp. 6448-6453; Wang, H. W. et al. (2002) Brain Res., vol. 924, no. 2, pp. 133-140; Klein, W. L. et al. (2001) Trends Neurosci., vol. 24, no. 4, pp. 219-224), suggesting that ADDLs bind to a receptor or receptor-like protein to trigger such a facile cellular response. Immunofluorescence detection with an ADDL-specific antibody localizes ADDL receptors on cultured nerve cells. Dissociated cultures of rat hippocampal cells were plated on laminin-coated coverslips (1.33×10³ cells/mm²). Cells were grown for 2-3 d. Cells were treated with 5 μM ADDLs in serum-free media for 1 h, rinsed, and fixed with 3.7% formaldehyde for 15 min. The cells were then rinsed with PBS, blocked with 10% NGS:PBS, and immunolabeled with 6E10-B (1:200) in NGS:PBS for 2 h at 37° C. followed by DTAF-streptavidin (1:333) in PBS for 1 h at 37° C. Cells were examined using MetaMorph imaging software. Binding is punctate and reminiscent of focal contact or receptor labeling. Punctate ADDL-binding occurs at numerous sites along the processes and cell bodies. (see FIG. 32)

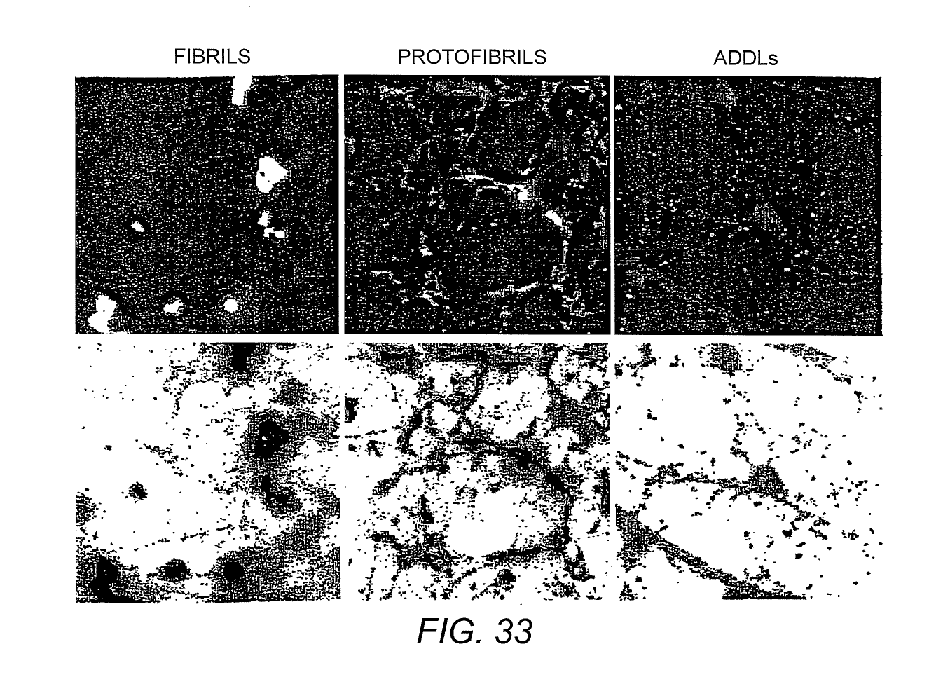

Furthermore, as shown in FIG. 33, punctate binding of ADDLs to nerve cells is distinct from the binding of amyloid protofibrils or fibrils. The assay is carried out as above, using preparations of Abeta (Aβ) that were fibrillar or protofibrillar.

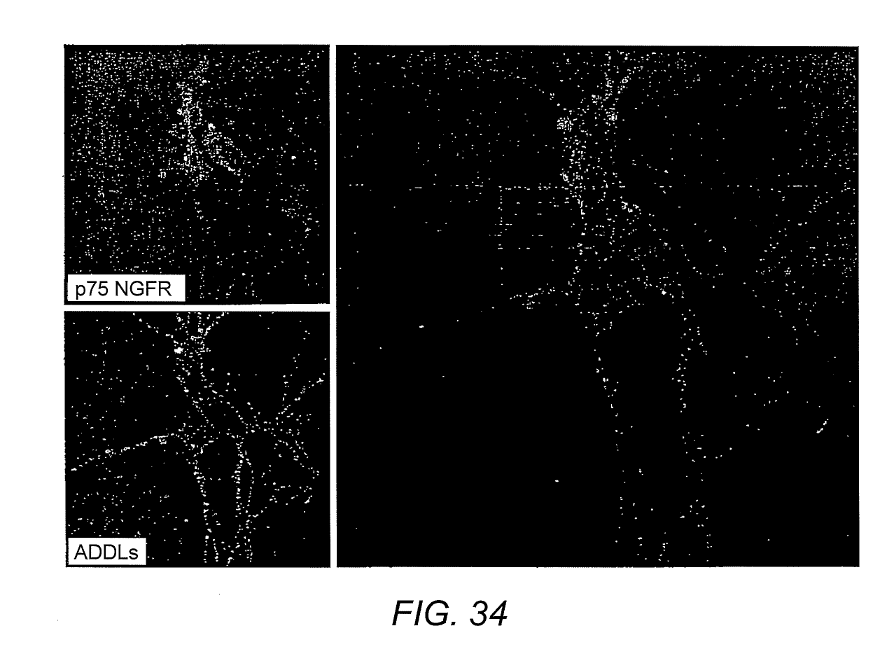

ADDLs bind to cell surface receptors that are distinct from the p75 nerve growth factor (NGF) receptor. (see FIG. 34) Previously, Abeta peptide of undefined structure had been shown to bind to NGF-r (p75) in neuroblastoma cells. (Kuner, P. et al. (1998) J. Neurosci. Res., vol. 54, no. 6, pp. 798-804). Immunofluorescence was used to determine whether ADDLs bind to the p75NGF-r or to distinct ADDL receptors on hippocampal nerve cells. Rat hippocampal cells were grown for 12 days. Cells were treated with 1 M ADDLs for 1.5 h at 37 C. Coverslips were rinsed once and then fixed with formaldehyde for 15 min. The coverslips were washed, permeabilized with 0.1% Triton X-100 in 10% NGS/PBS for 1.5 hrs, and labeled with monoclonal anti-NGF-r (1:100) and polyclonal anti-ADDL (M94-3)(1:500) at 4° C. overnight. Cells were then rinsed and incubated at room temp for 3 hours with AlexaFluor 594 anti-mouse and AlexaFluor 488 anti-rabbit (1:1000, each). Cells were rinsed and mounted with ProLong Anti-Fade medium prior to visualization using MetaMorph imaging software. As shown in FIG. 34, ADDL receptor complexes are clearly seen as puncta on hippocampal cell processes. NGF-r (p75 nerve growth factor receptor) was observed primarily around the cell body, demonstrating virtually no co-localization. This result contrasts with the findings of Kuner et al. (1998), indicating that ADDLs are distinct structures compared with the Abeta peptide used in experiments that demonstrated binding of Aβ to p75NGFR in a human neuroblastoma cell line.

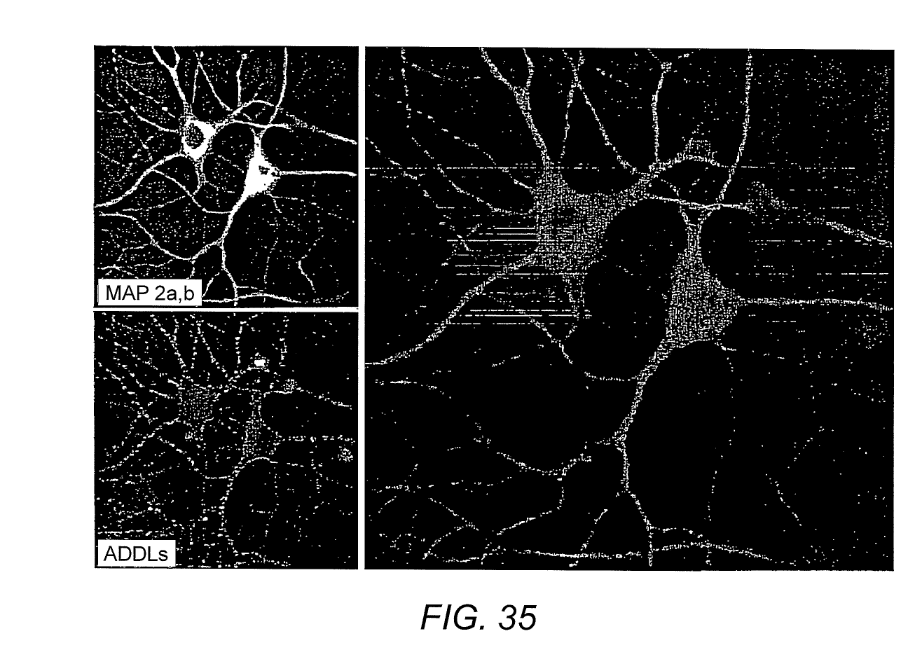

ADDL Receptors co-localize with MAP-2a,b staining, indicating dendritic localization. (see FIG. 35) Immunofluorescence can detect MAP2a,b localization and ADDL receptors on 12 day old rat hippocampal cells. Rat hippocampal cells are grown for 12 d. Cells are treated with 1 μM ADDLs for 1.5 h at 37° C. Coverslips are rinsed once and then fixed with formaldehyde for 15 min. The coverslips are washed, permeabilized with 0.1% Triton X-100 in 10% NGS/PBS for 1.5 h, and labeled with monoclonal anti-MAP2a,b (1:250) and polyclonal anti-ADDL (M94-3)(1:500) at 4° C. overnight. Cells are then rinsed and incubated at room temp for 3 h with AlexaFluor 594 anti-mouse and AlexaFluor 488 anti-rabbit (1:1000, each). Cells are rinsed and mounted with ProLong Anti-Fade medium prior to visualization using MetaMorph imaging software. As shown in FIG. 35, ADDLS are detected only on processes that stained with the anti-MAP2a,b antibody, suggesting that ADDLs bind primarily to dendrites, but not prevalently on axons.

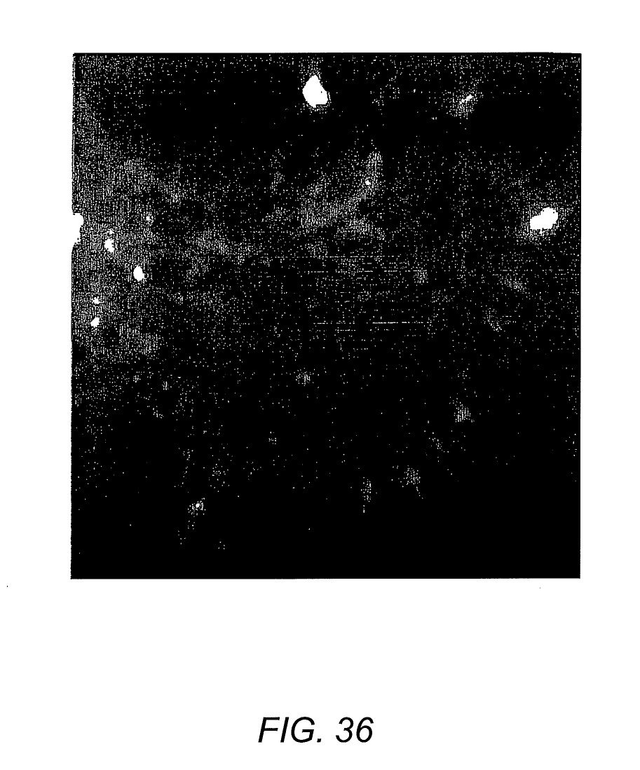

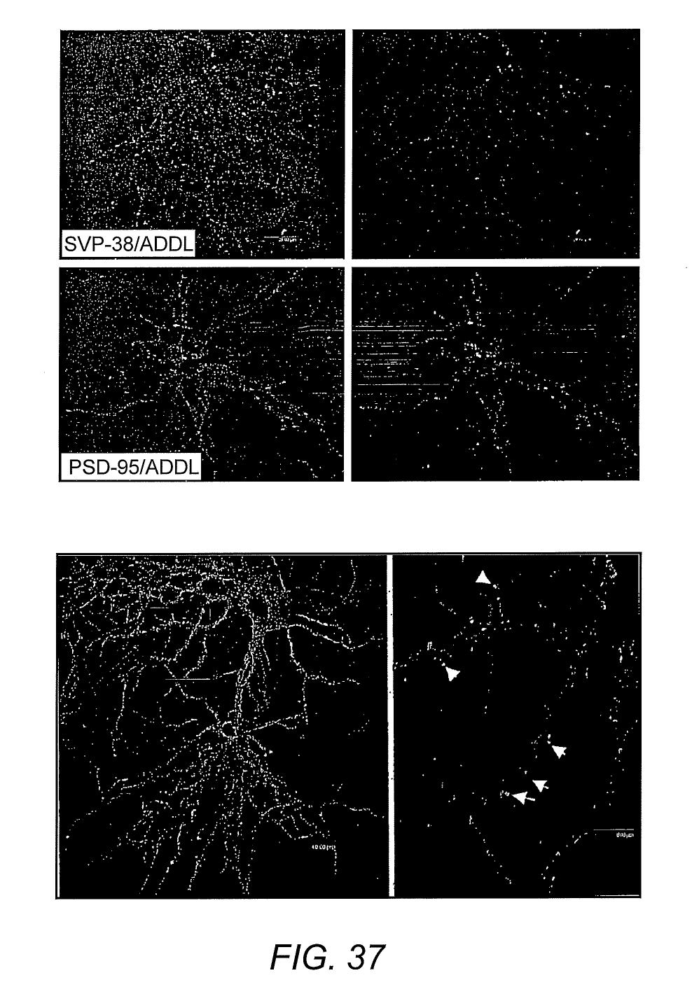

ADDLs bind to growth cones and lamellipodia tips. (see FIG. 36) Furthermore, as shown in FIG. 37, ADDL receptors localize on dendritic spines with the post-synaptic marker PSD-95 and CAM kinase II, with lower prevalence of localization at pre-synaptic terminals. Immunofluorescence of double labeled hippocampal neurons revealed that predominant localization of ADDL receptor complexes (green) occurs at post-synaptic sites (lower panels) identified by PSD-95 density (red). Significantly less co-localization occurs at pre-synaptic terminals identified by SVP-38 density (red-upper panel). Similar double labeling shows ADDL receptor complexes localize to dendritic spines with the post synaptic marker CAM kinase II.

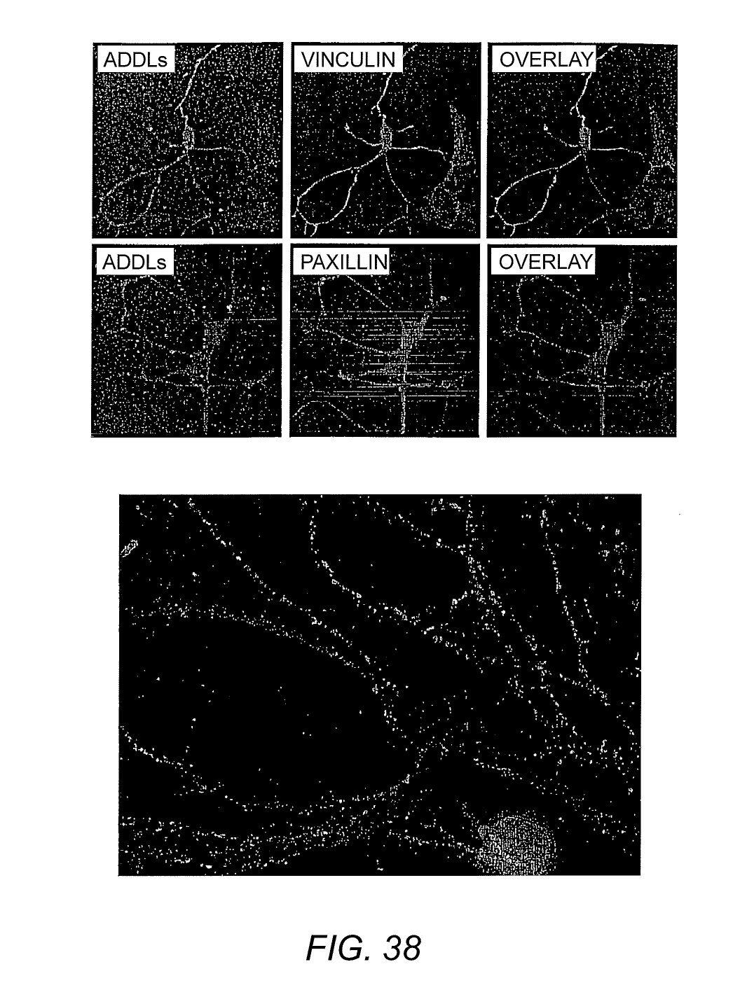

ADDL receptor puncta co-localize with paxillin and vinculin as components of neuronal focal adhesion contacts. (see FIG. 38) Previous studies demonstrated that treatment of rat neuroblastoma cells with ADDLs caused rapid phosphorylation of paxillin, with no change in vinculin phosphorylation (Berg, M. M. et al. (1997) J. Neurosci. Res., vol. 50, no. 6, pp. 979-989). To characterize further the signaling processes and molecules involved in ADDL receptor signaling, immunofluorescence can be used to determine whether ADDL receptor complexes co-localize with and paxillin and/or vinculin in hippocampal nerve cells.

Rat hippocampal cells are grown for 12 d. Cells are treated with 1M ADDLs for 1.5 h at 37 C. Coverslips are rinsed once and then fixed with formaldehyde for 15 min. The coverslips are washed, permeabilized with 0.1% Triton X-100 in 10% NGS/PBS for 1.5 h, and labeled with monoclonal anti-paxillin or anti-vinculin (1:100) and polyclonal anti-ADDL (M94-3) (1:500) at 4° C. overnight. Cells are then rinsed and incubated at room temp for 3 h with AlexaFluor 594 anti-mouse and AlexaFluor 488 anti-rabbit (1:1000, each). Cells are rinsed and mounted with ProLong Anti-Fade medium prior to visualization using MetaMorph imaging software.

ADDL receptor binding results in formation of distinct puncta on hippocampal cell processes (as routinely observed) and occasionally on cell bodies. (see FIG. 38) Paxillin is found on processes and cell bodies. ADDL receptor complexes appears to co-localize with paxillin only in a few instances. Vinculin is found predominantly at junctions between cell processes and at putative focal contact points, and ADDL receptor complexes can be detected at the majority of these focal contact sites. These observations suggest that ADDL receptors have characteristics very similar to adhesion receptors, the liganded complexes of which localize to focal contacts.

To confirm the minimal localization with paxillin, another assay is carried out in hippocampal nerve cultures prepared from E18 rat embryos. Neurons are treated at 26 d in culture with 1 μM ADDLs or equivalent volume of vehicle as a control for 1 h at 37° C. in hippocampal media. Cells are rinsed, fixed with 3.7% formaldehyde, washed with PBS 3× and then blocked with 10% NGS:PBS for 60 min. Coverslips are incubated for overnight at 8° C. with either PBS:NGS or anti-paxillin (1:100) and M90-2 anti ADDL polyclonal rabbit antibody (1:250) in PBS:NGS. Cells are rinsed with PBS 3× and then incubated with AlexaFluor 488 (green) anti-rabbit (1:1000) and biotinylated anti-mouse (1:250) in PBS:NGS for 1 h at 37° C. Cells are rinsed with PBS 3× and then incubated with AlexaFluor 594 (red) streptavidin (1:1000) in PBS:NGS for 1 h at 37° C. Cells are rinsed with PBS and then mounted with ProLong. Cells are imaged with a Nikon microscope and MetaMorph Imaging software. (see FIG. 38, bottom panel)

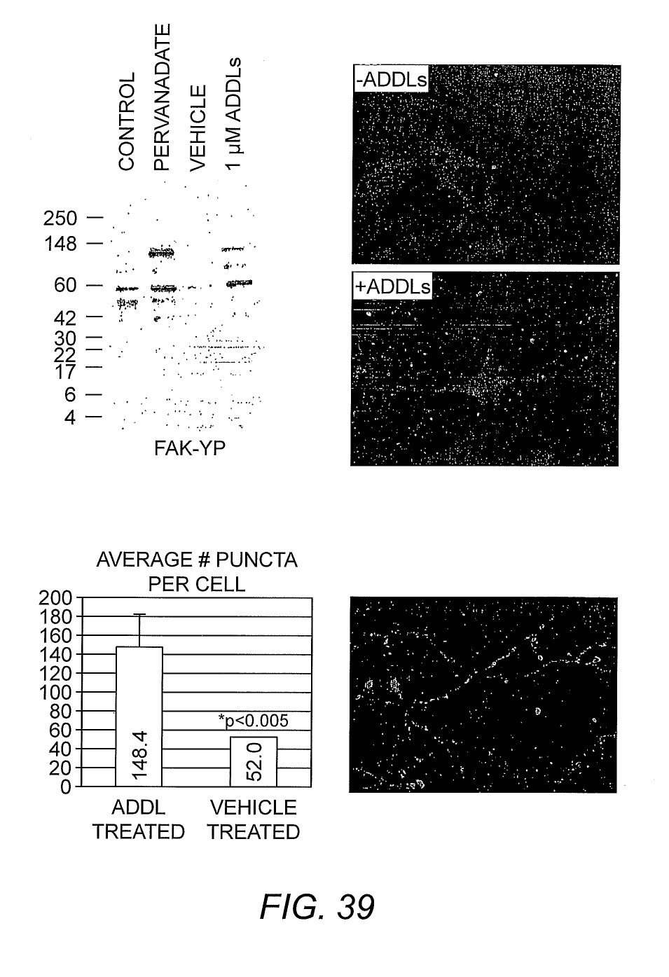

ADDL receptor binding activates phosphorylation of focal adhesion kinase (FAK) on a tyrosine, and ADDL receptor complexes localize with the phosphorylated FAK (FAK-YP). (see FIG. 39) Previous studies had demonstrated that mixed aggregates of Aβ1-42 could induce the phosphorylation of FAK (Zhang et al., 1994), and several assays are carried out to determine whether ADDL treatment and subsequent receptor complex formation could increase FAK phosphorylation.

Hippocampal cells are plated in 60 mm dishes at a concentration of 2 million cells/dish and allowed to grow for 5 d. Cells are treated with ADDLs (1 μM) or vehicle for 1 h or pervanadate (final concentrations: sodium orthovanadate 0.1 mM and H₂O₂ 3 mM in the culture medium) or PBS for 20 min at 37° C. Cells are rinsed with warm PBS briefly and lysed with 0.15 mL boiling lysis buffer (1% SDS, 1.0 mM sodium orthovanadate, 10 mM Tris pH 7.4). Cells are scraped and collected into a large microfuge tube and frozen overnight. Samples are thawed the following day and boiled for 5 min. Samples are spun at high speed for 1 min and the supernatants transferred to a new tubes. Protein concentration is determined using the Coomassie Plus kit. 4-20% Tris-HCl gels are loaded with Multimark standards and 10 μg protein for each of the hippocampal cell lysates in 5× Laemmli buffer with β-mercaptoethanol (BME), repeating so that the gels could be cut in half after transfer. Gels are run at 120V for ˜1.5 h, transferred to Immobilon-P PVDF for 1.5 h at 100 v at 8° C., blocked with 1% BSA-TBST overnight, incubated with FAK-YP antibody (clone 14) for 2 h at room temperature in 1% BSA-TBST, then incubated with HRP-anti-mouse secondary for 1 h at RT in 1% BSA-TBST. Bands are visualized using the SuperSignal West Femto kit and the Kodak Image Station, capturing images at 15 min intervals.

As shown in FIG. 39, FAK-YP is detected in all samples, and as expected, was prominent in the samples treated with pervanadate, a general inhibitor of phosphatases. ADDL treatment for 1 h also leads to a significant increase in FAK-YP. The FAK-YP antibody detects bands at ˜60 kDa and ˜85 kDa and ˜140 kDa.

To measure the increase and localization of FAK-YP triggered by ADDL receptor binding, hippocampal cells are treated with ADDLs and analyzed by immunofluorescence (FIG. 39, top left). The hippocampal cultures are prepared from E18 rat embryos and treated at 26 d in culture with 1 μM ADDLs or equivalent volume of vehicle as a control for 1 h at 37° C. in hippocampal media. Cells are rinsed, fixed with 3.7% formaldehyde, washed with PBS 3× and then blocked with 10% NGS:PBS for 60 min. Coverslips are incubated overnight at 8° C. with either PBS:NGS or FAK-YP (1:100) and M90-2 anti ADDL polyclonal rabbit antibody (1:250) in PBS:NGS. Cells are rinsed with PBS 3× and then incubated with AlexaFluor 488 (green) anti-rabbit (1:1000) and biotinylated anti-mouse (1:250) in PBS:NGS for 1 h at 37° C. Cells are rinsed with PBS 3× and then incubated with AlexaFluor 594 (red) streptavidin (1:1000) in PBS:NGS for 1 h at 37° C. Cells are rinsed with PBS and then mounted with ProLong. Cells are imaged with a Nikon microscope and MetaMorph Imaging software.

ADDL receptor binding causes a three-fold increase in the number of FAK-YP puncta detected by immunofluorescence after treatment for 1 h with 11M ADDLs. The cell average increases from 52 puncta to 148 puncta, and was accompanied by a 25% increase in puncta size and a 22% increase in spherical volume. (see FIG. 39)

### Example 25

### Therapeutic Antibodies

The use of antibodies to sequester amyloid beta peptide monomer or to clear fibrillar amyloid plaques has been proposed by a number of investigators. These methods do not target ADDLs, the most potent neurotoxic amyloid structures identified to date and the structures now recognized to be the likely cause of AD and memory deficits. In order for an antibody to be an effective therapeutic for AD and related memory deficit disorders, it must bind specifically to oligomers with no significant binding affinity for Aβ monomer and no significant binding affinity for amyloid fibrils, and it must be a human or humanized antibody with some ability to penetrate into the brain. The binding of the optimal antibody also must result in blockage of ADDL toxicity. If a potential therapeutic antibody has poor specificity, i.e. binding monomer in addition to oligomers, large fractions of administered antibody will be engaged by monomer, which is not neurotoxic, diminishing the levels of antibody available to bind and block the actions of the potent neurotoxic oligomers (ADDLs). If a potential therapeutic antibody cross-reacts with fibrils, in addition to binding monomer, then the antibody can bind to amyloid fibrils within deposited plaques, resulting in persistent inflammatory responses in the brain caused by antibody-plaque complexes that are not easily cleared from the brain.

Previously disclosed antibodies (M93-3 & M93-4) are polyclonal rabbit antibodies that exhibited preferential binding to ADDLs, but still exhibited fibril cross-reactivity and slight monomer binding. These antibodies were useful for demonstrating the effect of potent blockage of ADDL toxicity, however, these antibodies would not be useful for human therapeutics. New monoclonal antibodies are now disclosed, which have the ability to bind only oligomer structures, with no binding to monomer and no binding to fibrils.

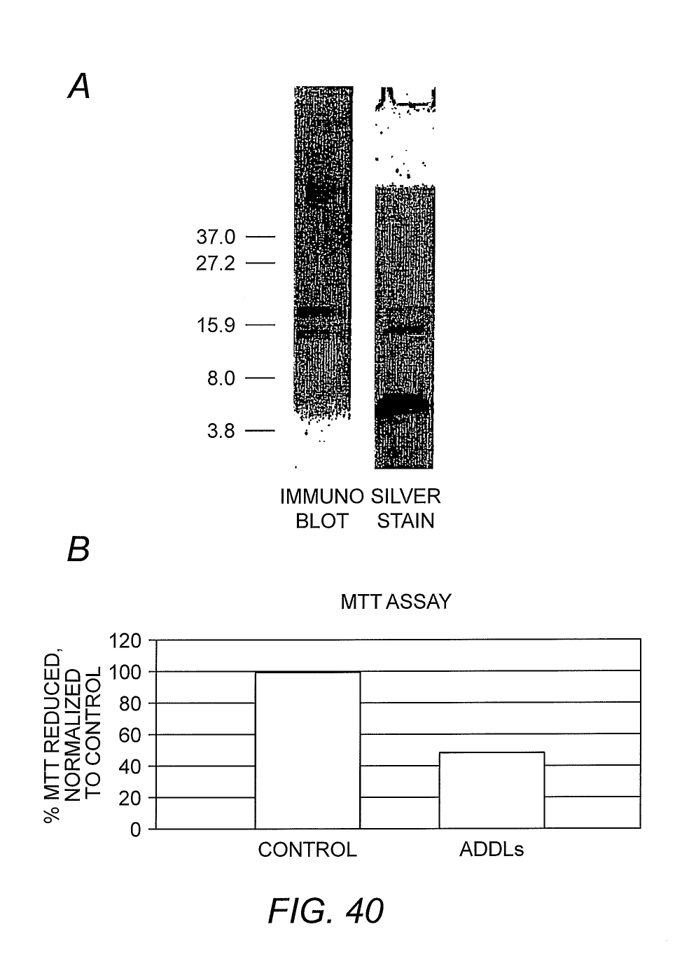

Injection of fibrillar Aβ causes plaque removal and prevents loss of memory in Tg mice that model AD (Bacskai, B. J. et al. (2002) J. Neurosci., vol. 22, no. 18, pp. 7873-7878; Jantzen, P. T. et al. (2002) J. Neurosci., vol. 22, no. 6, pp. 2246-2254; Bard, F. et al. (2000) Nat. Med., vol. 6, no. 8, pp. 916-919; Games, D. et al. (2000) Ann. NY Acad. Sci., vol. 920, pp. 274-284; Masliah, E. et al. (1996) J. Neurosci., vol. 16, no. 18, pp. 5795-5811). However, when this antigen was used in human trials, the trials had to be stopped due to brain inflammation. Since ADDL injection produces oligomer-selective polyclonal antibodies in rabbit, it appeared feasible that monoclonal antibodies might be generated in mice that would target epitopes found only on the small oligomeric ADDL forms. We predicted that antibodies against small molecular weight oligomers probably would not target plaques and thus may not cause a general inflammation reaction. Consequently, we injected ADDLs (see FIG. 40 for quality control of structure and toxicity of typical antigen) into three Balb/c mice every three weeks for six months. The injections averaged 92 μg total Aβ/animal/injection.

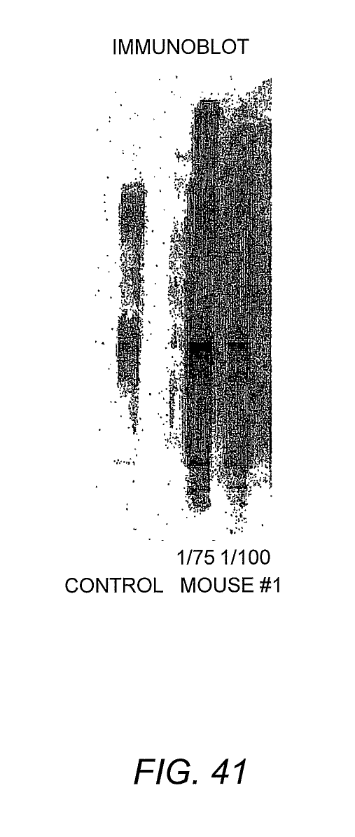

The results depicted in FIG. 41 are a typical response shown by the immunized mice after 6 months of injections. Antibodies appear to bind to monomer, trimer, tetramer, and some higher molecular weight material near the 12-24mer range. This result is in harmony with previous unpublished work in which we have found that TG mice modeling AD injected with 147 ug total AB (9 times) produce an immune response (24 out of 24 animals). In additio, our recent results with 6 out of 6 rabbits and 2 out of 2 chickens show that they produce polyclonal antibodies to injected ADDLs (˜150 ug total AB/animal for 6 injections). Characteristics of our first polyclonal antibody have recently been published (Lambert, M. P. et al. (2001) J. Neurochem., vol. 79, no. 3, pp. 595-605).

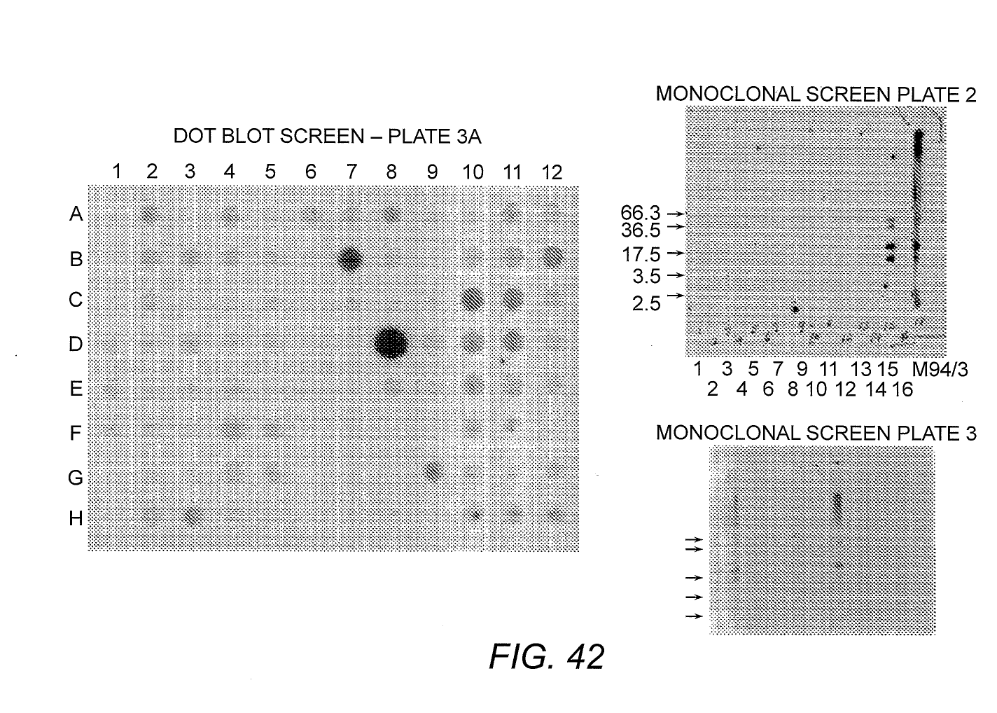

After fusion of the mouse spleen with SP2 myeloma cells, the resulting hybridomas are plated into 20 96-well plates and then tested for their ability to bind to 5 pmol ADDLs in a dot blot assay. Results from a typical assay are shown in FIG. 42, left. Screening is performed twice to allow for different rates of growth of the hybridomas. Dot blot assays on hybridoma fusion products with two separate mice spleen show that ˜11% of the hybridoma supernates in each case bind with strong intensity to ADDLs at 5 pmol.

From the highly positive dot blots, over 200 wells are screened in the immunoblot assay utilizing approximately 20 pmol ADDLs/lane (FIG. 42, right). One of the rabbit polyclonal antibodies is used as a positive control in every immunoblot. From these data, 41 hybridomas are selected to expand in a 24-well plate format.

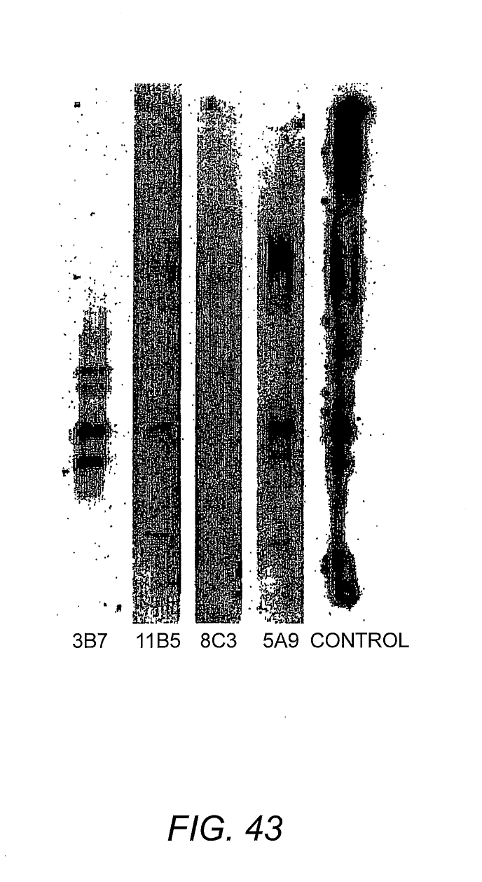

From the immunoblot assays, several interesting ADDL binding profiles were found (FIG. 43). In particular, one hybridoma, 3B7, appears to possess a strong reactivity only for the smaller molecular weight material. Others (8C3) may target only the 12-24-mer. 5A9 and 11B5 bind to lower and higher molecular weight species. Western blots also show several hybridoma supernates that bind with a smear to everything in the lane. Whether this is due to a non-specific binding or can be explained by dilution of the antibody to reduce its affinity has not been determined. Subcloning of the hybridomas is in progress.

An important attribute of certain monoclonal antibodies is their ability to identify their antigen in immunohistochemical protocols. Since we have shown in previous work that the oligomer-selective rabbit polyclonal antibodies do recognize ADDL binding sites on cultured cells (Lambert, Viola), it was important to determine if the hybridoma supernates also recognize ADDL binding sites on cells and compare them to the ones visualized by M94/3. Accordingly, ADDLs were incubated with 21-day hippocampal cultures and supernate from 3B7 was used to localize the binding. Results show that ADDLs bind to cultured hippocampal cells in small puncta, primarily on neurites. The images are very similar to those produced with the rabbit polyclonal antibody, although the puncta may be slightly smaller in the 3B7 image. The binding is very clean, as seen by the lack of signal in the vehicle image. Supernate from a non-reactive hybridoma (in the immunoblot, 14D3) also showed no reaction in the immunohistochemical assay.

### Example 26

### Prevention, Treatment and Diagnosis of ADDL-induced Disease

This aspect of the present invention pertains to the fields of medicine, medical diagnostics, molecular biology, cellular biology and biochemistry. Specifically, this aspect of the invention pertains to the diagnosis, prevention and treatment of degenerative diseases, especially neurodegenerative diseases such as Alzheimer's disease, mild cognitive impairment, Down's syndrome-related dementia, and other impaired memory disorders. More specifically, this aspect of the invention pertains to vaccines, antibodies, inhibitors and diagnostic reagents and methods specifically related to amyloid beta (β)-derived diffusible ligands (ADDLs) and the treatment, prevention and/or detection of disease states caused by ADDLs, including Alzheimer's disease, mild cognitive impairment, Down's syndrome related cognitive deficits, and inflammation.

The most common form of dementia and cognitive impairment in older individuals is Alzheimer's disease, for which a definitive diagnosis can be confirmed only at autopsy by measurement of hallmark senile plaques and neurofibrillary tangles. Over the past decade, many researchers have invoked the “amyloid cascade hypothesis”, to explain AD. This hypothesis argues that plaques and their constituent amyloid fibrils cause the neurodegeneration that leads to AD (Hardy, J. A. & Higgins, G. A. (1992) Science, vol. 256, pp. 184-185), but it fails to explain many contradictory aspects of AD symptoms and pathology, such as the poor spatial correlation between plaques and degenerated nerve cells.

Transgenic animal models overexpressing Aβ₁₋₄₂ have provided confirmation of the involvement of Aβ₁₋₄₂, but some of these transgenic mice exhibited profound cognitive deficits without depositing any plaques or amyloid fibrils.

The discovery of novel, soluble oligomeric Aβ₁₋₄₂ neurotoxins known as amyloid β-derived diffusible ligands (ADDLs) (Krafft, G. A. et al. (1997) U.S. patent application Ser. No. 08/796,089; Krafft, G. A. et al. (2001) U.S. Pat. No. 6,218,506; Lambert, M. P. et al. (1998) Proc. Natl. Acad. Sci. USA, vol. 95, pp. 6448-6453) provided a clear explanation for cognitive deficits linked to elevated Aβ₁₋₄₂, without the need or involvement of amyloid fibrils or plaques. Within the past year, the original author of the “amyloid cascade hypothesis” has reported that ADDLs, not fibrils, are the likely causative molecular pathogens in AD.

U.S. patent application Ser. No. 08/796,089 included data implicating ADDLs as potent neurotoxins capable of interfering with essential learning and memory processes, and it claimed methods for treatment and prevention of AD and cognitive disorders comprising interference with ADDL formation or activity. In this application, data are presented in support of methods for treatment, prevention and diagnosis of AD and related ADDL-induced disorders. These methods capitalize on recently discovered molecules capable of specific binding to ADDLs, and with no detectable binding to amyloid b monomer, and no detectable binding to fibrillar or protofibrillar aggregates of amyloid b. The highly specific nature of these molecules, including monoclonal antibody molecules, qualifies them to be highly effective therapeutic and preventative agents by virtue of their ADDL-blocking ability, and highly effective diagnostic reagents by virtue of their specific ADDL-detection in brain tissue (post-mortem), and in serum or cerebrospinal fluid (pre-mortem).

Because ADDLs can be detected in the serum, they can be claimed as a biomarker that correlates with cognitive health. The specific ADDL-binding molecules can thus be used for quantitative detection of ADDLs in serum as a function of time, providing a method for monitoring the effectiveness of any therapeutic molecule or dietary supplement in reducing the serum ADDL concentration, and documenting the correlative improvement of cognitive function associated with reduction of ADDL concentrations. This method can be applied to animal models of AD for characterization of potential AD therapeutics, and it can be applied to human clinical trials of potential AD and cognitive impairment therapeutics. This method can be incorporated into a laboratory diagnostic product to measure for the presence of ADDLs in blood, providing a basis for physicians to prescribe therapeutic agents that lower the level of ADDLs, or that lower the production of amyloid b, which comprises ADDLs. This method also can be incorporated into a consumer-friendly diagnostic product to measure for the presence of ADDLs in blood, providing a basis for the consumer to consume nutritional supplements containing naturally occurring substances that are known to be capable of blocking ADDL formation.

Also described and claimed are nutritional supplements and other components that are, which are useful in lowering the serum concentrations of ADDLs, as measured by diagnostic methods involving the ADDL-specific binding molecules.

These specific ADDL-binding molecules are also useful as imaging agents for in vivo detection of ADDLs that are bound to the surface of nerve cells in the brain. These imaging agents include reagents useful for positron emission tomography (PET), for magnetic resonance imaging or for any other imaging method that relies upon the specific localization of ADDLs and the detection of that localization made possible by attaching a reporting molecule such as a radiolabel or magnetic contrast agent to the ADDL-specific binding molecule.

These specific ADDL-binding molecules are also useful for discovering the specific receptor proteins on nerve cells that mediate the neurotoxic actions of ADDLs. In this application, the properties and characteristics of such ADDL-specific neuronal receptor proteins are also disclosed, and methods for discovering therapeutic and preventative agents that interfere with ADDL binding to these receptor proteins are also disclosed.

These specific ADDL-binding molecules are also useful in the discovery of small molecule drugs that interfere with ADDL formation or ADDL activity. Molecules that prevent ADDL formation are effective for prevention of the neurotoxic actions of ADDLs, and the presence of such ADDL formation blocking molecules can be confirmed using the specific ADDL-binding molecules to verify that ADDLs have not formed from amyloid β monomer.

### Example 27

### Alzheimer-Affected Brain: Presence of Oligomeric Aβ Ligands Provides a Molecular Basis for Reversible Memory Loss

Memory deterioration in Alzheimer's disease (AD) has been considered progressive and irreparable. However, remarkable recovery of memory function recently was reported for a transgenic model of Alzheimer's disease (AD) after mice were vaccinated with antibodies against amyloid β peptide (Aβ). Because amyloid plaques were unaffected, this model strongly links memory loss to soluble assemblies of Aβ. In various models, soluble oligomeric assemblies of Aβ are recognized as potent CNS neurotoxins whose neurological impact includes the rapid, non-degenerative blockade of synaptic information storage (long-term potentiation). As disclosed herein, such Aβ oligomers are present in human brain and increase as much as 70-fold in Alzheimer's disease. Aβ oligomers (also designated as ADDLs) act as ligands for cell surface proteins expressed in hippocampus and cerebrum but not cerebellum, suggesting a basis for the particular vulnerability of cognitive brain regions to AD. Results provide strong evidence that ADDLs are a significant factor in AD pathogenesis and constitute promising targets for new therapeutic drugs and antibodies that could reverse memory dysfunction.

Alzheimer's disease (AD) is a fatal, progressive dementia for which the earliest manifestation is memory failure. There is no cure for AD and its molecular basis is not yet established. Considerable evidence, however, indicates the disease is a proteinopathy linked to neurotoxic assemblies of the 42 amino acid peptide amyloid β (Aβ) (1, 2).

Aβ is an amphipathic molecule derived proteolytically from a transmembrane precursor protein (APP) (3). Strongly self-associating (4), the largest Aβ assemblies constitute the insoluble amyloid fibrils found in AD plaques (5, 6). Similar amyloid fibrils assemble from synthetic peptide in vitro. Synthetic preparations that contain conspicuous fibrils are neurotoxic (7, 8), but pure monomer solutions are not, indicating that toxicity requires self-assembly. A role for Aβ-derived neurotoxins in AD pathogenesis is strongly indicated by the elevated Aβ₁₋₄₂ common to disparate AD-linked mutations and risk factors (9). For many years, the requisite structure for toxicity and pathogenesis was thought to be the insoluble amyloid fibril (8), but despite seemingly strong support for the amyloid hypothesis, no consensus has emerged regarding its validity for AD. A major obstacle has been the poor correlation between dementia and amyloid plaque burden (10), frequently recapitulated in hAPP transgenic mice models of AD (11, 12).

Recent data, moreover, cast doubt on whether fibrils and associated cell death are required for memory loss (13). In hAPP transgenic mice, memory loss is preventable by Aβ vaccination, a remarkable effect that does not require elimination of amyloid deposits (14). Even more strikingly, vaccination with anti-Aβ monoclonal antibodies enables hAPP mice to recover lost memory function. Recovery happens within a day of injection (15) and occurs without impact on insoluble amyloid fibrils. Memory recovery in this model contradicts the widely-held view that AD is simply degenerative and irreversible (16), but recovery had been predicted by an alternative hypothesis for the structure and pathogenic role of Aβ-derived toxins (12, 17). In this alternative hypothesis, a basis for reversible, fibril-independent memory loss lies in the neurological properties of soluble Aβ assemblies. Distinct from fibrillar amyloid, these small globular oligomers (known as “ADDLs” (17)) and the somewhat larger, rod-shaped protofibrils (18, 19) are potent CNS neurotoxins (20, 21). The oligomers are especially relevant to memory dysfunction because they rapidly and selectively inhibit long-term potentiation (17, 22, 23), an established paradigm for synaptic information storage.

Based on their impact in model systems, it is clear that soluble Aβ assemblies would be an important factor in AD, if present. As disclosed herein, soluble Aβ assemblies indeed occur in human brain and increase as much as 70-fold in AD. Mirroring the structure of synthetic oligomers, the AD-linked molecules act as high-affinity ligands for cell surface proteins expressed in cognitive centers. Accumulation of oligomeric Aβ ligands in AD-affected brains is strong evidence for a pathogenic role, putatively accounting for the discrepancies between dementia and amyloid plaque burden, and it suggests their neutralization would provide a means to reverse memory loss.

### Materials:

Amyloid beta (Aβ₁₋₄₂) peptide was from American Peptide (Sunnyvale, Calif.), California Peptide Research (Napa, Calif.), or Recombinant Peptide, Inc. (Athens, Ga.). Hams F12 medium phenol red-free was from BioSource International (Camarillo, Calif.). Hibernate™ was from Life Technologies (Gaithersburg, Md.). Neurobasal™, horse serum, and B27 supplements™ were from Invitrogen (Carlsbad, Calif.). All other cell culture reagents were from Mediatech (Herndon, Va.). Unless otherwise indicated, chemicals and reagents were from Sigma-Aldrich (St. Louis, Mo.). The Cell Proliferation (MTT) kit was from Roche Boehringer Mannheim (Indianapolis, Ind.). The Coomassie Plus and BCA protein assays and the SuperSignal West Femto chemiluminescence kit were from Pierce (Rockford, Ill.). SDS-PAGE 4-20% Tris-Glycine gels, 2-D strips, and buffers were from BioRad (Hercules, Calif.). Hybond™ ECL™ nitrocellulose and HRP-conjugated secondary were from Amersham Biosciences (Piscataway, N.J.). Oligomer-selective antibodies (M93,M94) were produced and characterized earlier(24). Alexa Fluor® 488-conjugated secondary antibody was from Molecular Probes (Eugene, Oreg.). Timed pregnant Sprague Dawley rats were obtained from Charles River Laboratories, Inc. (Wilmington, Mass.). Samples of frontal cortex and cerebellum from AD and age-matched control brains were obtained from the Northwestern Alzheimer's Disease Center Neuropathology Core and stored at −80° C. until used.

### Synthetic ADDLs:

ADDLs were prepared according to published protocols (24, 25) and as described herein.

### Cell Culture:

Hippocampal cells were prepared and maintained according to Brewer (26) using poly-lysine (0.002%) coated coverslips plated at a density of 1.8×10⁴ cells/cm² in Neurobasal™ with B27 supplements and L-glutamine (2.5 μM). Cortical and cerebellar cells were cultured as previously described (27) with cerebellar cultures given higher KCl (25 mM). Cells were exposed to 5 μM Ara-C for 24 h, followed by 24 h at 2.5 μm Ara-C. For assays of metabolic activity, cells were plated onto poly-L-lysine-coated 24-well plates at a density of 0.4×10⁶ cells/well. When ADDLs were added, medium was changed to F12 medium with 50 nM or 100 nM synthetic ADDLs (plus 25 mM KCl for cerebellar cultures) and metabolic activity (MTT reduction) measured after 48 h using the Cell Proliferation kit according to manufacturer's instructions.

### Immunocytochemistry:

Cultures were rinsed once with culture media and fixed with 3.7% formaldehyde. The coverslips were washed, permeabilized with 0.1% Triton X-100 in 10% normal goat serum and phosphate buffered saline (NGS:PBS) for 90 minutes at room temperature (RT), immunolabeled with polyclonal M94-3 antibody (1:500) overnight at 4° C., followed by an incubation with Alexa Fluor® 488 anti-rabbit (2 μg/mL) for ˜3 hours at RT. The cells were rinsed, mounted with ProLong® reagent, and visualized using MetaMorph imaging software (Universal Imaging Corp, West Chester, Pa.).

### Membrane Preparation:

All manipulations of human and adult rat brain tissues were performed at 4° C.

Cerebellum, cortex, and hippocampus were homogenized in 20 vol. Buffer A (PBS, pH 7.4, 0.32 M sucrose, 50 mM HEPES, 25 mM MgCl₂, 0.5 mM dithiothreitol, 200 μg/mL PMSF, 2 μg/mL pepstatin A, 4 μg/mL leupeptin, and 30 μg/mL benzamidine hydrochloride) and centrifuged at 1,000×g for 10 min. The pellet was re-homogenized in 10 vol. Buffer A and centrifuged again. The combined supernatants were centrifuged at 100,000×g for 1 h and the pellet was used for total membrane fraction.

### Soluble Tissue Extracts:

Frontal cortex from AD or control brain (0.2 g) was homogenized in 20 vol. F12 containing protease inhibitors (as above) and centrifuged at 100,000×g for 1 h. The pellet was re-homogenized in 10 volumes F12+ protease inhibitors and re-centrifuged. The protein concentration of the combined supernatants was determined. An aliquot of protein (4 mg) was then concentrated to a volume 60 μL or less, using a Centricon™-10 concentrator.

### Two-Dimensional Gel Electrophoresis:

Proteins of soluble cortical tissue extracts were separated according to published procedures using Bio-Lytes pH 3-10 carrier ampholytes (28). Synthetic ADDLs, 1 nmol in 10 μL F12, were treated exactly as cortex and stained with silver as previously described (24).

### Immunoassays:

Ligand blots were based on published procedures (29). Membrane preparations were extracted with detergent (30) for 15 min on ice, then solubilized proteins were separated by SDS-PAGE for 3-4-h at 120 v and transferred to nitrocellulose. Blots incubated with TBST containing 5% nonfat dry milk overnight, washed 3 times with cold F12 medium, and incubated with 10 nM ADDLs for 3 h at 4-8° C. After washing away unbound material with TBST, bound ADDLs were labeled with M93/3 (1:1000) and visualized with enhanced chemiluminescence. Immunoblots and dot blots were carried out as previously described (24, 29).

### Assay for Assembled Forms of Soluble Aβ:

Because oligomers, if present in human brains, might be non-abundant, we first obtained an antibody known to detect Aβ in western blots at femtomole levels (31) (kindly provided by Dr. Potempska). This antibody, however, proved selective for monomers. To gain the required sensitivity and selectivity, we generated antibodies by vaccinating rabbits with full-length monomers and oligomers of Aβ₁₋₄₂ (24). FIG. 25A illustrates the specificity of the two antibodies, tested with identical solutions. The predominant oligomer first to appear is tetramer, although continuing incubations give stable, non-fibrillar assemblies up to 24-mers (25). Dot blot immunoassays with the new antibody also showed selectivity for oligomers (FIG. 25B). While solutions of Aβ monomer (i.e., freshly treated with hexafluoro-isopropanol; HFIP) required 1 pmol for a signal (FIG. 25B, top), solutions with oligomer (i.e., not monomerized by HFIP) showed immunoreactivity at 1 fmol (total Aβ; FIG. 25B, bottom). This assay was linear over at least a 100-fold range and gave consistent replicates (see e.g., FIG. 29B), making the assay suitable for comparative analyses of brain extracts.

### Soluble Aβ Assemblies in Human Brain: Large Increases in Alzheimer's Disease:

Dot blot assays were used to test for assembled forms of Aβ in soluble human brain extracts, comparing frontal cortex of five AD patients with age-matched controls. Brain tissue was homogenized in detergent-free nerve cell culture medium (sans serum) in an effort to preserve in vivo conditions. Supernatants from 100,000 g×60 minutes spins were applied to filters for dot blot immunoassays. Immunoreactivity was robust in AD brain extracts, but near background for controls (FIG. 26). Essentially identical results were obtained in three separate trials. Population averages for AD brain were 12-fold higher than control brain (p<0.001 for data shown). In the control group, one sample was elevated 10-fold compared to the low readings. Compared to the lowest three controls, the three highest AD samples showed a 70-fold increase of soluble Aβ assemblies.

To characterize the soluble Aβ assemblies detected in dot-blot analyses and to verify the absence of fibrils, we analyzed brain extracts by two-dimensional electrophoresis and immunoblotting (using the quaternary structure-sensitive antibody as before). No fibrillar material was evident. Instead, there was a prominent oligomer at approximately 56 kDa and pI 5.6 (FIG. 27A). In contrast, high-speed pellets from AD brain (amyloid fraction) contained copious immunoreactive material throughout the isoelectric focusing dimension, none of which entered the size-separating dimension (not shown). Control tissue showed no immunoreactive material (FIG. 27C). Oligomers prepared from synthetic Aβ (FIG. 27B) matched the properties of soluble oligomers from AD brain (putatively 12-mers; MW=53 +/−4 kDa for 3 subjects). Other experiments have shown a range of oligomers up to 24 mer can form in synthetic preparations (25), and detergent extraction of brain tissue released occluded oligomers (4-mers and 24-mers in addition to 12-mers; not shown) that are within this range. The matching of size and pI, along with mutual recognition by assembly-dependent antibodies, indicates oligomers prepared from synthetic Aβ or obtained from AD brain tissue were structurally equivalent.

### Aβ Oligomers are Ligands for Proteins in Membrane Rafts:

Aβ oligomers from AD brain have size and solubility consistent with a predicted capacity for ligand activity (12, 17). We therefore examined tested Aβ oligomers in a ligand overlay assay, which can assess specificity of protein-protein interactions (29, 32). Rat brain membrane proteins were separated by SDS-PAGE, transferred, and incubated with extracts. The presence of Aβ oligomers in AD, but not controls, was verified by dot blots (FIG. 29B). Binding was detected by antibodies as before. Disease-dependent binding was evident, with AD oligomers acting as ligands for three membrane-associated binding proteins (p100, p140, and p260; FIG. 29A). The binding proteins were much less abundant than other membrane proteins (Coomassie blue staining, not shown), consistent with highly selective ligand interactions. Synthetic oligomers showed selective binding to the same proteins (top right). Whether the greater binding of ligands in crude AD extract to p100 might be due to additional proteins (or complexes) absent from pure synthetic preparations is not yet known. p140 and p260 were enriched in fractions that contained rafts (top right), which are membrane domains specialized for signal transduction (33). Raft localization was consistent with resistance of p140 and p260 to Triton X-100 solubilization (not shown).

### Properties of Binding Proteins Parallel Vulnerability to Aβ Ligands:

Because human and synthetic Aβ oligomers were similar in structure and binding, the next experiments were carried out only with synthetic ligands, which also conserved human samples. To test for selective expression of binding proteins in rat brain, we compared two regions typically damaged in AD (hippocampus, cerebrum) with a region that is not (cerebellum). Hippocampus and cerebrum, but not cerebellum, contained p140 and p260 (FIG. 28, top left). p100 did not show selective expression. In these assays, the immunoreactivity was proportional to membrane protein added (not shown), and a control for non-specific antibody binding (FIG. 28, top middle) established that it was ligand-dependent.

Human brain expressed the same binding proteins as rat (FIG. 28, top right), as did mouse and pig (not shown). Expression again was higher in cortex than cerebellum. Relative abundance of p260 and p140 binding sites in 5 normal elderly compared to 5 AD samples (FIG. 28, bottom left) indicated a trend toward lower levels in AD (p<0.05 for p260, and p<0.01 for p140 using Student's T-test), but overlap was evident between the populations. The results, however, were consistent with occurrence of oligomer binding proteins on cells vulnerable to degeneration in AD. This possibility was supported by toxicity assays. Cortical cultures (and hippocampus, not shown) were sensitive to oligomers, whereas cerebellar cultures were not (FIG. 28, bottom right inset). Oligomers at 50 nM were maximally effective in this assay, which monitors both metabolism and vesicle trafficking (34, 35). Ligand binding to cortical p140 occurred at approximately commensurate doses (FIG. 28, bottom right).

### Hot Spots of Binding to Cultured Neurons:

Consistent with the overlay results, previous data from flow cytometry indicated that synthetic oligomers bind with specificity to cell surface proteins (17). This conclusion was confirmed and extended by immunofluorescence microscopy (see FIG. 32). Cultured hippocampal neurons were incubated with oligomers for 5 minutes, washed, and immunolabeled. Whether oligomers were from human brain extracts or made from synthetic Aβ, they showed the same highly selective patterns of attachment (FIG. 32, Panels A & C). Binding was at small puncta (˜0.2-0.5 μm across), which overlapped in size with signal transduction specializations such as focal contacts, synaptic spines, or clustered rafts. Control extracts gave no puncta, consistent with ligand overlay results (FIG. 32, Panel B) Controls without ADDLs or without primary antibody also showed no immunoreactivity. For synthetic oligomers, the puncta were evident at concentrations as low as 20 nM (total Aβ). Some puncta were on cell bodies but predominantly occurred on neurites. Immunoreactivity was detectable without cell permeabilization, indicating puncta were at the plasma membrane. Puncta were also observed on cortical, but not on cerebellar, neurons (not shown), in harmony with the overlay and toxicity results.

Findings presented here provide evidence that memory loss in AD is caused by small soluble oligomers of Aβ. These CNS neurotoxins previously were shown in animal and cell experiments to selectively inhibit mechanisms of synaptic information storage (17, 22, 23). Selective immunoassays, capable of discriminating low levels of oligomers within a milieu of abundant monomer, have verified the presence of oligomeric Aβ ligands in AD and have established that AD is linked to major increases in these neurotoxins. Results here substantiate the importance of Aβ to AD pathogenesis, provide an explanation for the long-standing problem that disease correlates poorly with plaques, and provide an impetus to develop new approaches to AD therapeutics that specifically target these soluble neurotoxins.

Assays in well-established model systems previously have implicated soluble A3-derived neurotoxins in memory dysfunction. Active vaccination of hAPP mice using Aβpreparations revealed that memory dysfunction could be ameliorated without elimination of plaques (14), suggesting possible involvement of toxic assemblies of Aβ that were soluble. In a striking extension of this concept, passive vaccination of hAPP mice using an Aβ monoclonal antibody recently was shown to bring about recovery of impaired memory function (15). Recovery is fast, within one day of vaccination, and it occurs without impact on levels of insoluble amyloid deposits. This antibody-mediated recovery of memory is evidence for the role of Aβ oligomers, whose impact on memory earlier had been predicted to be reversible (2, 12, 17). Soluble Aβ assemblies in memory-deficient hAPP mice have been detected in preliminary findings (36) and could comprise oligomers or protofibrils, each of which is soluble and neuroactive (17, 20, 21). As yet, however, only oligomers have been reported to block synaptic plasticity (LTP), a cellular paradigm for memory processes. Oligomers, when introduced into animals (2, 23) or hippocampal tissue slices (17, 22), selectively inhibit LTP within a few minutes; greater exposure of neurons to oligomers, in terms of cell surface as well as time, leads to selective nerve cell death. A key finding of the current work is the demonstration that oligomers previously shown to be neurologically disruptive in experimental models have counterparts in human brain affected with AD. Analogous neurological impact of these oligomers in human brain could account for the poor correlation between plaque abundance and AD.

The large increase in oligomers in AD (up to 70-fold) indicates a nonlinear dependence on monomer concentration, which only increases 2-3 fold (37). Non-linearity might reflect the chemistry of oligomerization, although it also is possible that oligomers accumulate in complexes with high-affinity binding proteins such as seen in overlay assays. It is intriguing that our early results suggest that even some individuals without plaques exhibit elevated levels of oligomers. This finding is consistent with the ability of stable oligomers to form in vitro sans large amyloid fibrils (17, 25), and it suggests that oligomers may begin to play a role in the earliest stages of the disease, perhaps even in pre-Alzheimer's memory dysfunctions.

The mechanism by which oligomers block synaptic plasticity is unknown. One hypothesis previously suggested (2) is that LTP inhibition derives from displacement of Fyn. This synaptically-localized Src-family protein tyrosine kinase is implicated in LTP (38) and in the activity of Aβ-derived neurotoxins (17), and it is associated with Alzheimer's pathology (39). Displacement of Fyn could preclude phosphorylation of particular targets coupled to LTP such as the ERK-CREB pathway (40). Supporting this possibility, CREB activation is inhibited by non-degenerative doses of Aβ under conditions that give oligomers (41). A related hypothesis is that oligomers disrupt plasticity-related vesicle trafficking and insertion of critical proteins into synaptic membranes. Glutamate receptor insertion is associated with LTP and with reversal of long-term depression (LTD), both of which are inhibited by oligomers (22); LTP-induced insertion of receptors into synaptic membranes is Src-family-dependent (42). The ability of Aβ toxins to alter vesicle transport has been shown in experiments with cell lines and fibrillar preparations (34).

Although the relationship of oligomer binding proteins to toxic mechanisms has not been established, these binding proteins along with Fyn are enriched in membrane rafts. Rafts are domains specialized for signal transduction and trafficking (33, 43, 44), and they play a role in organization of synapse components such as nicotinic acetylcholine receptors (45). The possibility that a member of the nicotinic receptor family, some of which are linked to Fyn (46), might be an oligomer binding protein is under investigation. The fact that oligomers bind to differentially expressed proteins is in harmony with the hypothesis that vulnerability of neurons to Alzheimer's disease is receptor-mediated. Consistent with this hypothesis, AD-vulnerable brain regions (hippocampus, cerebrum) show responses to oligomers and express oligomer binding proteins, whereas the AD-insensitive cerebellum neither responds (12) nor expresses oligomer binding proteins.

Highly selective ligand activity is consistent with oligomer solubility and structure. Soluble oligomers presumably present hydrophilic surfaces with amino acid sequences capable of specific protein-protein interactions. Because the ligands are homo-oligomers, these interactions could impact more than one binding protein, analogous, e.g., to trophic factors such as insulin or BDNF (47, 48), or extracellular matrix proteins such as laminin (49). The punctate pattern of binding for oligomers differs significantly from that reported for protofibrils, which appear to coat cell surfaces (20). Thus, although there is an indication that PFs are present in CSF (50), the binding seen here for extracted human ligands and synthetic oligomers indicates little contribution from PFs, consistent with the two-dimensional gel analyses.

It has become clear that formation of non-fibrillar toxic oligomers from Aβ represents an archetype for a general property of amyloidogenic proteins (51). Various amyloidogenic proteins other than Aβ now have been shown to form granular, non-fibrillar assemblies in the earliest stages of self-association, and, as first seen for Aβ, these non-fibrillar assemblies can be cytotoxic. Some, such as Parkinson's-related alpha-synuclein, are disease-associated (52). In other cases (e.g., prions) it has not been determined if the oligomers contribute to pathogenesis. An interesting aspect of prion assembly, however, is that its oligomerization is off-pathway with respect to prion fibrillogenesis (53). We do not know if Aβoligomerization is analogously off-pathway. Aβ oligomers present unique epitopes absent from fibrils and as such they can be used to develop safe therapeutic antibodies for human use. Antibodies that target only soluble toxins should provide the memory benefits shown in the transgenic mice study, but without the serious inflammation found in recent AD vaccine trials (54), which were designed to eliminate plaques. If, as shown by the transgenic mouse study (15), memory recovery derives from antibody neutralization of toxic oligomeric ligands outside the blood brain barrier, the possibilities are even more promising.

### REFERENCES

1. Hardy, J. & Selkoe, D. J. (2002) Science 297, 353-356.
2. Klein, W. L. (2000) in Molecular Mechanisms of Neaurodegenerative Diseases, ed. Chesslet, M.-F. (Humana Press, Totowa), pp. 1-49.
3. Kang, J., Lemaire, H. G., Unterbeck, A., Salbaum, J. M., Masters, C. L., Grzeschik, K. H., Multhaup, G., Beyreuther, K. & Muller-Hill, B. (1987) Nature 325, 733-736.
4. Parbhu, A., Lin, H., Thimm, J. & Lal, R. (2002) Peptides 23, 1265-1270.
5. Glenner, G. G., Wong, C. W. (1984) Biochem. Biophys. Res. Conmnun. 120, 885-90.
6. Masters, C. L., Simms, G., Weinman, N. A., Multhaup, G., McDonald, B. L. & Beyreuther, K. (1985) Proc. Natl. Acad. Sci. U.S. A 82, 4245-4249.
7. Pike, C. J., Burdick, D., Walencewicz, A. J., Glabe, C. G. & Cotman, C. W. (1993) J. Neurosci. 13, 1676-1687.
8. Lorenzo, A. & Yankner, B. A. (1994) Proc. Natl. Acad. Sci. U.S. A 91, 12243-7.
9. Ertekin-Taner, N., Graff-Radford, N., Younkin, L. H., Eckman, C., Baker, M., Adamson, J., Ronald, J., Blangero, J., Hutton, M. & Younkin, S. G. (2000) Science 290, 2303-2304.
10. Terry, R. D. (1999) in Alzheimer disease, eds. Terry, R. D., Katzman, R., Bick, K. L., & Sisodia, S. S. (Lippincott Williams, and Wilkins, pp. 187-206.
11. Mucke, L., Masliah, E., Yu, G. Q., Mallory, M., Rockenstein, E. M., Tatsuno, G., Hu, K., Kholodenko, D., Johnson-Wood, K. & McConlogue, L. (2000) J. Neurosci. 20, 4050-4058.
12. Klein, W. L., Krafft, G. A. & Finch, C. E. (2001) Trends Neurosci. 24, 219-224.
13. Westerman, M. A., Cooper-Blacketer, D., Mariash, A., Kotilinek, L., Kawarabayashi, T., Younkin, L. H., Carlson, G. A., Younkin, S. G. & Ashe, K. H. (2002) Journal of Neuroscience 22, 1858-1867.
14. Morgan, D., Diamond, D. M., Gottschall, P. E., Ugen, K. E., Dickey, C., Hardy, J., Duff, K., Jantzen, P., DiCarlo, G., Wilcock, D. et al. (2000) Nature 408, 982-985.
15. Dodart, J. C., Bales, K. R., Gannon, K. S., Greene, S. J., DeMattos, R. B., Mathis, C., DeLong, C. A, Wu, S., Wu, X, Holtzman, D. M. et al. (2002) Nat. Neurosci. 5, 452-457.
16. Small, G. W., Rabins, P. V., Barry, P. P., Buckholtz, N. S., DeKosky, S. T., Ferris, S. H., Finkel, S. I., Gwyther, L. P., Khachaturian, Z. S., Lebowitz, B. D. et al. (1997) JAMA 278, 1363-1371.
17. Lambert, M. P., Barlow, A. K., Chromy, B. A., Edwards, C., Freed, R., Liosatos, M., Morgan, T. E., Rozovsky, I., Trommer, B., Viola, K. L. et al. (1998) Proc. Natl. Acad. Sci. U.S. A 95, 6448-6453.
18. Harper, J. D., Wong, S. S., Lieber, C. M. & Lansbury, P. T. (1997) Chem. Biol. 4, 119-125.
19. Walsh, D. M., Lomakin, A., Benedek, G. B., Condron, M. M. & Teplow, D. B. (1997) J. Biol. Chem. 272, 22364-22372.
20. Hartley, D. M., Walsh, D. M., Ye, C. P., Diehl, T., Vasquez, S., Vassilev, P. M., Teplow, D. B. & Selkoe, D. J. (1999) J. Neurosci. 19, 8876-8884.
21. Walsh, D. M., Hartley, D. M., Kusumoto, Y., Fezoui, Y., Condron, M. M., Lomakin, A., Benedek, G. B., Selkoe, D. J. & Teplow, D. B. (1999) J. Biol. Chem. 274, 25945-25952.
22. Wang, H. W., Pasternak, J. F., Kuo, H., Ristic, H., Lambert, M. P., Chromy, B., Viola, K. L., Klein, W. L., Stine, W. B., Krafft, G. A. et al. (2002) Brain Res. 924, 133-140.
23. Walsh, D. M., Klyubin, I., Fadeeva, J. V., Cullen, W. K., Anwyl, R., Wolfe, M. S., Rowan, M. J. & Selkoe, D. J. (2002) Nature 416, 535-539.
24. Lambert, M. P., Viola, K. L., Chromy, B. A., Chang, L., Morgan, T. E., Yu, J., Venton, D. L., Krafft, G. A., Finch, C. E. & Klein, W. L. (2001) J. Neurochem. 79, 595-605.
25. Klein, W. L. Aβ toxicity in Alzheimer's disease: globular oligomers (ADDLs) as new vaccine and drug targets. Neurochemistry International In Press. 2002.
26. Brewer, G. J., Torricelli, J. R., Evege, E. K. & Price, P. J. (1993) J. Neurosci. Res. 35, 567-576.
27. Samdani, A. F., Newcamp, C., Resink, A., Facchinetti, F., Hoffman, B. E., Dawson, V. L. & Dawson, T. M. (1997) J. Neurosci. 17, 4633-4641.
28. Friso, G. & Wikstrom, L. (1999) Electrophoresis 20, 917-927.
29. Denda, S., Reichardt, L. F. & Muller, U. (1998) Mol. Biol. Cell 9, 1425-1435.
30. Zhang, C., Lambert, M. P., Bunch, C., Barber, K., Wade, W. S., Krafft, G. A. & Klein, W. L. (1994) J. Biol. Chem. 269, 25247-25250.
31. Potempska, A., Mack, K., Mehta, P., Kim, K. S. & Miller, D. L. (1999) Amyloid. 6, 14-21.
32. Bowe, M. A., Mendis, D. B. & Fallon, J. R. (2000) J. Cell Biol. 148, 801-810.
33. Simmons, K. & Toomre, D. (2000) Nat. Rev. Mol. Cell. Biol. 1, 31-39.
34. Liu, Y., Peterson, D. A. & Schubert, D. (1998) Proc. Natl. Acad. Sci. U.S. A 95, 13266-13271.
35. Shearman, M. S., Ragan, C. I. & Iversen, L. L. (1994) Proc. Natl. Acad. Sci. U.S. A 91, 1470-1474.
36. Chang, L., Wang, Z., Bakhos, L., Venton, D. L. & Klein, W. L. (2002) Furfuryl amine substituted beta cyclodextrins inhibit ADDL formation and toxicity. Personal Communication
37. McLean, C. A., Chemy, R. A., Fraser, F. W., Fuller, S. J., Smith, M. J., Beyreuther, K., Bush, A. I. & Masters, C. L. (1999) Ann. Neurol. 46, 860-866.
38. Grant, S. G. & Silva, A. J. (1994) Trends Neurosci. 17, 71-75.
39. Shirazi, S. K. & Wood, J. G. (1993) Neuroreport 4, 435-437.
40. Ying, S. W., Futter, M., Rosenblum, K., Webber, M. J., Hunt, S. P., Bliss, T. V. & Bramham, C. R. (2002) J. Neurosci. 22, 1532-1540.
41. Tong, L., Thornton, P. L., Balazs, R. & Cotman, C. W. (2001) J. Biol. Chem. 276, 17301-17306.
42. Grosshans, D. R., Clayton, D. A., Coultrap, S. J. & Browning, M. D. (2002) Nat. Neurosci. 5, 27-33.
43. Li, X., Galli, T., Leu, S., Wade, J. B., Weinman, E. J., Leung, G., Cheong, A., Louvard, D. & Donowitz, M. (2001) J. Physiol 537, 537-552.
44. Chamberlain, L. H., Burgoyne, R. D. & Gould, G. W. (2001) Proc. Natl. Acad. Sci. U.S.A. 98, 5619-5624.
45. Bruses, J. L., Chauvet, N. & Rutishauser, U. (2001). Neurosci. 21, 504-512.
46. Kihara, T., Shimohama, S., Sawada, H., Honda, K., Nakamizo, T., Shibasaki, H., Kume, T. & Akaike, A. (2001) J. Biol. Chem. 276, 13541-13546.
47. Ottensmeyer, F. P., Beniac, D. R., Luo, R. Z. & Yip, C. C. (2000) Biochemnistiy 39, 12103-12112.
48. Ibanez, C. F., Ilag, L. L., Murray-Rust, J. & Persson, H. (1993) EMBO J. 12, 2281-2293.
49. Marangi, P. A., Wieland, S. T. & Fuhrer, C. (2002) J. Cell Biol. 157, 883-895.
50. Pitschke, M., Prior, R., Haupt, M. & Riesner, D. (1998) Nat. Med. 4, 832-834.
51. Bucciantini, M., Giannoni, E., Chiti, F., Baroni, F., Formigli, L., Zurdo, J., Taddei, N., Ramponi, G., Dobson, C. M. & Stefani, M. (2002) Nature 416, 507-511.
52. Volles, M. J., Lee, S. J., Rochet, J. C., Shtilerman, M. D., Ding, T. T., Kessler, J. C. & Lansbury, P. T., Jr. (2001) Biochemistiy 40, 7812-7819.
53. Baskakov, I. V., Legname, G., Baldwin, M. A., Prusiner, S. B. & Cohen, F. E. (2002) J. Biol. Chem. 277, 21140-21148.
54. Birmingham, K. & Frantz, S. (2002) Nat. Med. 8, 199-200.

### Example 28

### ADDL Binding Molecules and Uses Thereof

The most common form of dementia and cognitive impairment in older individuals is Alzheimer's disease, for which a definitive diagnosis can be confirmed only at autopsy by measurement of hallmark senile plaques and neurofibrillary tangles. Over the past decade the “amyloid cascade hypothesis” has been used frequently to explain AD. This hypothesis argues that plaques and their constituent amyloid fibrils cause the neurodegeneration that leads to AD (Hardy, J. A. & Higgins, G. A. (1992) Science, vol. 256, pp. 184-185), but it fails to explain many contradictory aspects of AD symptoms and pathology, such as the poor spatial correlation between plaques and degenerated nerve cells. Transgenic animal models overexpressing Aβ₁₋₄₂ have provided confirmation of the involvement of Aβ₁₋₄₂, but some of these transgenic mice exhibited profound cognitive deficits without depositing any plaques or amyloid fibrils. Aβ₁₋₄₂ is a 42-amino acid amphipathic peptide derived proteolytically from a widely expressed membrane precursor protein (Selkoe, D. J. (1994) Annu. Rev. Neurosci., vol. 17, pp. 489-517). As a monomer, the amyloid peptide has never been demonstrated to have toxic effects, and in some studies it has been purported to have neurotrophic effects.

Monomers of Aβ₁₋₄₂ assemble into at least three neurotoxic species: fibrillar amyloid (Pike, C. J. et al. (1993) J. Neurosci., vol. 13, pp. 1676-1687; Lorenzo, A. & Yanker, B. A. (1994) Proc. Natl. Acad. Sci. USA, vol. 91, pp. 12243-12247), protofibrils (Hartley, D. M. et al. (1999) J. Neurosci., vol. 19, pp. 8876-8884; Walsh, D. M. et al. (1999) J. Biol. Chem., vol. 274, pp. 25945-25952, and Aβ₁₋₄₂-derived diffusible ligands (ADDLs) (Lambert, M. P. et al. (1998) Proc. Natl. Acad. Sci. USA, vol. 95, pp. 6448-6453). Fibrillar amyloid is insoluble, and deposits of fibrillar amyloid are easily detected in AD and transgenic mice because of their birefringence with dyes such as thioflavin S. Fibrillar amyloid is a major protein component of senile plaques in Alzheimer's disease brain. Aβ peptides of various lengths, including Aβ1-40, 1-42, 1-43, 25-35, and 1-28 assemble into fibrils in vitro. All of these fibrils have been reported to be toxic to neurons in vitro and to activate a broad range of cellular processes. Hundreds of studies describe Aβ fibril neurotoxicity, but numerous studies also describe poor reproducibility and highly variable toxicity results. The variability has been attributed, in part, to batch-to-batch differences in the starting solid peptide and these differences relate specifically to the various physical or aggregation states of the peptide, rather than the chemical structure or composition. Protofibrils are large yet soluble meta-stable structures first identified as intermediates en route to full-sized amyloid fibrils (Walsh, D. M. et al. (1997) J. Biol. Chem., vol. 272, pp. 22364-22372).

ADDLs comprise small soluble Aβ₁₋₄₂ oligomers, predominantly trimers and tetramers but also higher-order species (Lambert, M. P. et al. (1998) Proc. Natl. Acad. Sci. USA, vol. 95, pp. 6448-6453; Chromy, B. A. et al. (2000) Soc. Neurosci. Abstr., vol. 26, p. 1284). All three forms of assembled Aβ₁₋₄₂ rapidly impair reduction of the dye MTT (Shearman, M. S. et al. (1994) Proc. Natl. Acad. Sci. USA, vol. 91, pp. 1470-1474; Walsh, D. M. et al. (1999) J. Bio. Chem., vol. 274, pp. 25945-25952; Oda, T. et al. (1995) Exp. Neurol., vol. 136, pp. 22-31), possibly the consequence of impaired vesicle trafficking (Liu, Y. & Schubert, D. (1997) J. Neurochem., vol. 69, pp. 2285-2293), and they ultimately kill neurons (Longo, V. D. et al. (2000) J. Neurochem., vol. 75, pp. 1977-1985; Loo, D. T. et al. (1993) Proc. Natl. Acad. Sci. USA, vol. 90, pp. 7951-7955; Hartley, D. M. et al. (1999) J. Neurosci., vol. 19, pp. 8876-8884). All three forms also exhibit very fast electrophysiological effects. Amyloid and protofibrils broadly disrupt neuronal membrane properties, inducing membrane depolarization, action potentials, and increased EPSPs (Hartley, D. M. et al. (1999) J. Neurosci., vol. 19, pp. 8876-8884), while ADDLs selectively block long-term potentiation (LTP) (Lambert, M. P. et al. (1998) Proc. Natl. Acad. Sci. USA, vol. 95, pp. 6448-6453; Wang, H. et al. (2000) Soc. Neurosci. Abstr., vol. 26, pp. 1787; Wang et al. (2002), Brain Research 924, 133-140). ADDLs also show selectivity in neurotoxicity, killing hippocampal but not cerebellar neurons in brain slice cultures (Kim, H.-J. (2000) Doctoral Thesis, Northwestern University, pp. 1-169). Given the poor correlation between fibrillar amyloid and disease progression (Terry, R. D. (1999) in Alzheimer's Disease (Terry, R. D. et al., Eds.), pp. 187-206, Lippincott Williams & Wilkins), it is likely that fibrillar amyloid deposits are not the toxic form of Aβ₁₋₄₂ most relevant to AD. Non-fibrillar assemblies of Aβ occur in AD brains (Kuo, Y. M. et al. (1996) J. Biol. Chem., vol. 271, pp. 4077-4081; Roher, A. E. et al. (1996) J. Biol. Chem., vol. 271, pp. 20631-20635; Enya, M. et al. (1999) Am. J. Pathol., vol. 154, pp. 271-279; Funato, H. et al. (1999) Am. J. Pathol., vol. 155, pp. 23-28; Pitschke, M. et al. (1998) Nature Med., vol. 4, pp. 832-834) and these species appear to correlate better than amyloid with the severity of AD (McLean, C. A. et al. (1999) Ann. Neurol., vol. 46, pp. 860-866; Lue, L. F. et al. (1999) Am. J. Pathol., vol. 155, pp. 853-862). Soluble Aβ oligomers are likely to be responsible for neurological deficits seen in multiple strains of transgenic mice that do not produce amyloid plaques (Mucke, L. et al. (2000) J. Neurosci., vol. 20, pp. 4050-4058; Hsia, A. Y. et al. (1999) Proc. Natl. Acad. Sci. USA, vol. 96, pp. 3228-3233; Klein, W. L. (2000) in Molecular Mechanisms of Neurodegenerative Diseases (Chesselet, M.-F., Ed.), Humana Press; Klein, W. L. et al. (2001) Trends Neurosci., vol. 24, pp. 219-224).

The discovery ADDLs (Krafft et al., (1997) U.S. patent application Ser. No. 08/796,089; Krafft et al., (2001) U.S. Pat. No. 6,218,506; Lambert et al., 1998) now provides a clear explanation for cognitive deficits linked to elevated Aβ₁₋₄₂, without the need to invoke the involvement of amyloid fibrils or plaques as the cause of AD. Remarkably, several publications by Prof D. Selkoe, the original author of the “amyloid cascade hypothesis”, have reported on the neurotoxicity and LTP blocking ability of ADDLs, citing them as the likely causative molecular pathogens in AD, and as targets for effective therapeutic intervention. (Walsh, D. M., Selkoe, D. et al., (2002) Biochem Soc. 30, Walsh, D. M., Selkoe, D. et al., (2002) Nature 416, 535).

U.S. patent application Ser. No. 08/796,089 reported data implicating ADDLs as potent neurotoxins capable of interfering with essential learning and memory processes, and it claimed methods for treatment and prevention of AD and cognitive disorders comprising interference with ADDL formation or activity. This application expands these claims, disclosing molecules that bind specifically to ADDLs, molecules which enable methods for the diagnosis, monitoring, prevention and treatment of diseases associated with ADDLs, including AD, mild cognitive impairment and other memory deficit disorders.

ADDL assembly blockers were first disclosed by the present inventors in PCT/US98/02426, filed 5 Feb. 19989 and further examples were disclosed in U.S. patent application Ser. No. 09/369,236, filed 4 Aug. 1999, and in U.S. patent application Ser. No. 10/166,856, filed 11 Jun. 2002. It has been reported, and is verified herein, that certain extracts of ginko biloba are capable of preventing ADDL assembly. Polyclonal antibodies raised against ADDL immunogens also were shown to block ADDL toxicity (Lambert, M. et al. (2001) J. Neurochem., vol. 79, pp. 595-605), although probably not by blocking assembly.

Over the past 3 years, a novel therapeutic strategy for Alzheimer's disease has emerged, based on vaccination with aggregated Aβ preparations. The initial studies that utilized this approach involved transgenic AD model mice that were vaccinated with Aβfibrils, a procedure which was reported to afford some protection from behavioral deficits normally manifest in these mice (Schenk, D. (1999) Nature, vol. 400, pp. 173-177; Morgan D. G. et al. (2001) Nature, in press; Helmuth, L. (2000) Science, vol. 289, p. 375; Arendash, G. et al. (2000) Soc. Neurosci. Abstr., vol. 26, p. 1059; Yu, W. et al. (2000) Soc. Neurosci. Abstr., vol. 26, p. 497). This result was surprising because it had generally not been appreciated that effective immune protection could be conferred on the brain side of the blood brain barrier (BBB). Apparently the protective effects observed in these transgenic AD mouse vaccination studies resulted from direct transport of anti-amyloid antibodies across the blood brain barrier in sufficient quantities to reduce the levels of toxic amyloid structures. Alternatively, it is conceivable that antibodies circulating in the bloodstream were capable of binding and clearing amyloid in sufficient quantities to reduce brain levels and produce a beneficial symptomatic effect. Several of the Tg mouse vaccination studies reported that total brain amyloid levels had not been lowered significantly, compared with amyloid levels in unvaccinated Tg AD mice in the control groups, which raises doubts about the plausibility of the Aβ clearance mechanism.

In other studies, it was demonstrated that direct injection of anti-amyloid antibodies into the brains of transgenic AD mice resulted in a significant reduction in brain amyloid levels (Bard, F. et al. (2000) Nature Med., vol. 6, pp. 916-919), however this approach involved delivery of antibody levels significantly higher than could be expected from passive transport across the BBB.

Regardless of the operative mechanism in these vaccinated Tg AD mice, the promising behavior protection results provided ample impetus to move forward with human testing of a fibrillar Ab vaccine AN1792 by the Elan Corporation (Helmuth, L. (2000) Science, vol. 289, p. 375). Their successful Phase I safety studies led to initiation of Phase II efficacy studies in AD patients. Unfortunately, these Phase II studies were halted recently because 12 of 97 AD patients in the study had developed vaccine related complications involving brain inflammation and encephalitis. Although the specific reason(s) for these serious complications is not known definitively, it can be surmised that vaccination with Ab fibrils would generate a significant immune response to the amyloid plaques in the brain, and that this would result in persistent activation of microglial cells and production of inflammatory mediators, all of which would contribute to severe encephalitis. In fact, this glial activation mechanism is precisely the mechanism proposed to explain the efficacy of the Elan vaccine approach (Schenk, D. (1999) Nature, vol. 400, pp. 173-177). These sobering results now make it very clear that any successful immune strategy for prevention or therapy of AD, whether involving a vaccine or a therapeutic antibody, will require a much more selective approach that targets toxic structures directly and specifically.

One alternative proposed in the literature is to use therapeutic antibodies with defined safety characteristics, an approach that underlies the use of antibodies that bind to the monomer form of amyloid b peptide. Many of these antibodies might be expected to prevent assembly of monomeric Ab 1-42 into ADDLs by sequestering the monomer and/or sterically preventing critical assembly and folding pathways that lead to Ab oligomers. (Dodel, R, (2002) EP-01172378; 2002; Schenk, D B et al. (1999) US, 00322289 and (2000) WO-00072880; Chain, B (1999) US, 00169687, (2001) WO-00142306; Holtzman, D M et al. (2000) US, 00184601; Frangione, B et al. (2000) US, 00205578). However, therapeutic strategies involving administration of such monomer-binding antibodies will be expensive because significant quantities of antibody will be needed in order to lower monomer concentration sufficiently to suppress oligomer formation.

Other vaccines approaches based on fragments of Ab monomer also have been published and patented recently. The Ab monomer is not particularly immunogenic because it is a naturally occurring human protein sequence for which the majority of binding competent T-cells have been deleted to avoid auto immunity. Attempts to direct the human immune response towards Ab monomer epitopes will risk autoimmunity with the identical sequences that are naturally present within the APP sequence, which occurs on the surface of most cell types.

The generation or use of molecules or antibodies to bind and sequester oligomers was claimed in PCT/US98/02426, filed 5 Feb. 19989 and further examples were disclosed in U.S. patent application Ser. No. 09/369,236, filed 4 Aug. 1999, wherein the activity of ADDLs is blocked. Several recent references have described ideas similar to this, such as the use of cross-linked oligomers as immunogens or the use of oligomers themselves as immunogens. (Walsh, D. M., Selkoe, D. et al., (2002) Biochem Soc. 30; Bush, A et al. US, 00214779; Srivastava (2000), US, 00489219).

In this application, data are presented in support of methods for treatment, prevention and diagnosis of AD and related ADDL-induced disorders, based on molecules that bind specifically to ADDLs, molecules that disrupt ADDL assembly, and vaccines capable of focusing the immune response to produce ADDL-neutralizing antibodies that do not cross react with fibrils. These methods capitalize on recently discovered molecules capable of specific binding to ADDLs, with no detectable binding to amyloid b monomer, and with no detectable binding to fibrillar or protofibrillar aggregates of amyloid b. The highly specific nature of these molecules, including monoclonal antibody molecules, qualifies them to be highly effective therapeutic and preventative agents by virtue of their ADDL-blocking ability, and highly effective diagnostic reagents by virtue of their specific ADDL-detection in brain tissue (post-mortem), and in serum or cerebrospinal fluid (pre-mortem).

The present invention seeks to overcome the substantial problems with the prior art that are based largely on the flawed theory that amyloid fibrils and plaques cause AD. Accordingly, one object of the present invention is the production, characterization and use of new compositions comprising specific ADDL-binding molecules such as anti-ADDL antibodies, which are capable of direct or indirect interference with the activity and/or formation of ADDLs (soluble, globular, non-fibrillar oligomeric Aβ₁₋₄₂ assemblies). These and other objects and advantages of the present invention, as well as additional inventive features, will be apparent from the description herein.

The present invention pertains to amyloid beta-derived diffusible ligands (ADDLs), antibodies that bind to ADDLs (anti-ADDL antibodies), uses of anti-ADDL antibodies to discover anti-ADDL therapeutics, and uses of anti-ADDL antibodies in the diagnosis, treatment and prevention of diseases associated with ADDLs, including Alzheimer's disease, learning and memory disorders, and neurodegenerative disorders. The invention specifically pertains to antibodies that recognize and bind ADDLs preferentially, with no significant binding capability for monomer or fibril forms of the amyloid peptide. Antibodies with these characteristics are useful for blocking the neurotoxic activity of ADDLs, and they are useful for eliminating ADDLs from the brain via clearance of antibody-ADDL complexes. Such antibodies are also particularly useful for treatment and prevention of Alzheimer's disease and other ADDL-related diseases in patients where prevalent fibrillar amyloid deposits exist in the brain, and for whom treatment with antibodies that preferentially bind to fibrillar forms of amyloid will result in serious brain inflammation and encephalitis.

Monoclonal antibodies with these characteristics also are useful for detection of ADDLs in biological samples, including human plasma, cerebrospinal fluid, and brain tissue. Anti-ADDL antibodies are useful for quantitative measurement of ADDLs in cerebrospinal fluid, enabling the diagnosis of individuals adversely affected by ADDLs. Such adverse effects may manifest as deficits in learning and memory, alterations in personality, and decline in other cognitive functions such as those functions known to be compromised in Alzheimer's disease and related disorders. Anti-ADDL antibodies are also useful for quantitative detection of ADDLs in brain tissue obtained at autopsy, to confirm pre-mortem diagnosis of Alzheimer's disease.

The invention further pertains to the use of ADDLs to select or identify antibodies or any other ADDL binding molecule or macromolecule capable of binding to ADDLs, clearing ADDLs from the brain, blocking ADDL activities, or preventing the formation of ADDLs. Additional inventions include new composition of matter, such molecule being capable of selecting antibodies or anti-ADDL binding molecules, or inducing an ADDL blocking immune response when administered to an animal or human. The invention extends further to include such uses when applied to methods for creating synthetic antibodies and binding molecules and other specific binding molecules through selection or recombinant engineering methods as are known in the art.

Specifically, the invention pertains to the preparation, characterization and methods of using such anti-ADDL antibodies. The invention also pertains to the use of anti-ADDL antibodies for the detection of ADDL formation and for the detection of molecules that prevent ADDL formation. The invention further pertains to the use of such antibodies to detect molecules that block ADDL binding to specific ADDL receptors present on the surface of nerve cells that are compromised in Alzheimer's disease and related disorders.

ADDLs comprise amyloid β (Aβ) peptide assembled into soluble, globular, non-fibrillar, oligomeric structures that are capable of activating specific cellular processes. Disclosed herein are methods for preparing and characterizing antibodies specific for ADDLs as well as methods for assaying the formation, presence, receptor protein binding and cellular activities of ADDLs. Also described are compounds that block the formation or activity of ADDLs, and methods of identifying such compounds. ADDL formation and activity are relevant inter alia to compromised learning and memory, nerve cell degeneration, and the initiation and progression of Alzheimer's disease. Modulation of ADDL formation or activity thus can be employed according to the invention in the treatment of learning and memory disorders, as well as other diseases, disorders or conditions that are due to the effects of the ADDLs.

The invention pertains to new compositions of matter, termed amyloid beta-derived diffusible ligands or amyloid beta-derived dementing ligands (ADDLs). ADDLs consist of amyloid β peptide assembled into soluble non-fibrillar oligomeric structures that are capable of activating specific cellular processes. A preferred aspect of the present invention comprises antibodies and binding molecules that are specific for ADDLs, and methods for preparation, characterization and use of antibodies or binding molecules that are specific for ADDLs. Another preferred embodiment comprises antibodies or binding molecules that bind to ADDLs but do not bind to Aβ monomers or fibrillar aggregates. Another aspect of the invention consists of methods for assaying the formation, presence, receptor protein binding and cellular activities of ADDLs, and methods for diagnosing diseases or potential diseases resulting from the presence of ADDLs. A further aspect of the invention is the use of anti-ADDL antibody or anti-ADDL binding molecules for the therapy and/or prevention of Alzheimer's disease and other diseases associated with the presence of ADDLs. The invention further encompasses assay methods and methods of identifying compounds that modulate (e.g., increase or decrease) the formation and/or activity of ADDLs. Such compounds can be employed in the treatment of diseases, disorders, or conditions due to the effects of the ADDLs.

Because ADDLs can be detected in the serum, they represent a biomarker correlating with cognitive health. The specific ADDL-binding molecules can thus be used for quantitative detection of ADDLs in serum as a function of time, providing a method for monitoring the effectiveness of any therapeutic molecule or dietary supplement in reducing the serum ADDL concentration, and documenting the correlative improvement of cognitive function associated with reduction of ADDL concentrations. This method can be applied to animal models of AD for characterization of potential AD therapeutics, and it can be applied to human clinical trials of potential AD and cognitive impairment therapeutics. This method can be incorporated into a laboratory diagnostic product to measure for the presence of ADDLs in blood, providing a basis for physicians to prescribe therapeutic agents that lower the level of ADDLs, or that lower the production of amyloid b, which comprises ADDLs. This method also can be incorporated into a consumer-friendly diagnostic product to measure for the presence of ADDLs in blood, providing a basis for the consumer to consume nutritional supplements containing naturally occurring substances that are known to be capable of blocking ADDL formation.

Also described and claimed are nutritional supplements and other components that are, which are useful in lowering the serum concentrations of ADDLs, as measured by diagnostic methods involving the ADDL-specific binding molecules.

These specific ADDL-binding molecules are also useful as imaging agents for in vivo detection of ADDLs that are bound to the surface of nerve cells in the brain. These imaging agents include reagents useful for positron emission tomography (PET), for magnetic resonance imaging or for any other imaging method that relies upon the specific localization of ADDLs and the detection of that localization made possible by attaching a reporting molecule such as a radiolabel or magnetic contrast agent to the ADDL-specific binding molecule.

These specific ADDL-binding molecules are also useful for discovering the specific receptor proteins on nerve cells that mediate the neurotoxic actions of ADDLs. In this application, the properties and characteristics of such ADDL-specific neuronal receptor proteins are also disclosed, and methods for discovering therapeutic and preventative agents that interfere with ADDL binding to these receptor proteins are also disclosed. Such molecules that interfere with the binding of ADDLs to specific proteins on nerve cells are useful for preventing the blockage of LTP and preventing the blockage of information storage that are triggered by ADDLs, and thereby are effective molecules for the treatment of memory and cognitive deficits in diseases associated with ADDLs, such as Alzheimer's disease, mild cognitive impairment and Down's syndrome.

These specific ADDL-binding molecules are also useful in the discovery of small molecule drugs that interfere with ADDL formation or ADDL activity. Molecules that prevent ADDL formation are effective for prevention of the neurotoxic actions of ADDLs, and the presence of such ADDL formation blocking molecules can be confirmed using the specific ADDL-binding molecules to verify that ADDLs have not formed from amyloid b monomer.

Finally, new compositions are claimed that have the capability to generate antibodies in an immune response that are specific for neutralizing ADDLs. These new compositions are oligomers made from rapidly assembling peptides or peptidomimetics molecules, wherein the oligomers present certain epitopes to the immune system to trigger and ADDL-neutralizing responses.

All of the patents and patent applications as well as all other scientific or technical writings referred to herein are incorporated by reference to the extent that they are not contradictory.

The preceding description of the preferred embodiments of the invention is presented for purposes of illustration and description, and is not intended to be exhaustive or to limit the invention to the precise forms disclosed. The description is selected to best explain the principles of the invention and practical application of these principles to enable others skilled in the art to best utilize the invention in various other embodiments and with various modifications as are suited to the particular use contemplated. The scope of the invention shall not be limited by the specification, by shall be defined by the claims set forth herein.

What is claimed is:

1. A method for producing an amyloid beta-derived diffusible ligand (ADDL) neutralizing antibody comprising
(a) injecting ADDLs into an animal to induce an immune response to the ADDLs;
(b) isolating antibodies that bind to ADDLs;
(c) identifying antibodies of (b) that neutralize ADDL neurotoxicity thereby producing an ADDL neutralizing antibody.

2. The method of claim 1, wherein the ADDLs of step (a) are obtained by
(i) dissolving monomeric amyloid beta peptide in a solvent to form a stock solution;
(ii) diluting the stock solution; and
(iii) incubating the diluted stock solution so that ADDLs are formed.

3. The method of claim 2, wherein the monomeric amyloid beta peptide of step (i) is prepared by
(A) dissolving amyloid beta peptide in hexafluoroisopropanol; and
(B) removing the hexafluoroisopropanol to obtain monomeric amyloid beta peptide.

4. The method of claim 2, wherein the solvent of step (i) is dimethyl sulfoxide.

5. The method of claim 2, wherein step (ii) comprises diluting the stock solution in cell culture medium.

6. The method of claim 1, further comprising selecting antibodies that do not bind to monomeric amyloid beta peptide.

7. The method of claim 1, further comprising selecting antibodies that do not bind to amyloid fibrils.

8. The method of claim 1, further comprising selecting antibodies that do not bind to monomeric amyloid beta peptide or amyloid fibrils.

9. An amyloid beta-derived diffusible ligand (ADDL) neutralizing antibody produced by the method of claim 1.


SEQUENCE LISTING (14 sequences)

SEQ ID NO: 1 - DNA, 20 bp, Artificial sequence
  Forward rat IL-1beta primer
gcaccttctttcccttcatc

SEQ ID NO: 2 - DNA, 20 bp, Artificial sequence
  Reverse rat IL-1beta primer
tgctgatgtaccagttgggg

SEQ ID NO: 3 - DNA, 19 bp, Artificial sequence
  Forward rat GFAP primer
cagtccttgacctgcgacc

SEQ ID NO: 4 - DNA, 19 bp, Artificial sequence
  Reverse rat GFAP primer
gcctcacatcacatccttg

SEQ ID NO: 5 - protein, 42 aa, Homo sapiens [sequence omitted]

SEQ ID NO: 6 - protein, 5 aa, Homo sapiens
  MISC_FEATURE: portion of amyloid beta sequence that is necesssary for polymerization to occur
KVLFF

SEQ ID NO: 7 - protein, 6 aa, Homo sapiens
KLVFFA

SEQ ID NO: 8 - protein, 19 aa, Artificial sequence
  Synthetic beta-amyloid precursor protein (beta-APP peptide mutant
NVPGHERMGRGRTSSKELA

SEQ ID NO: 9 - protein, 11 aa, Artificial sequence
  Synthetic peptide capable of forming a beta-turn which enables the assembly of the peptide into oligomers
RXXXXXXXXXX

SEQ ID NO: 10 - protein, 36 aa, Artificial sequence
  Synthetic peptide capable of assembling into oligomers
DSGYEVQQQKLVFFAEDVGSNKGAIIGLMVGGVVIA

SEQ ID NO: 11 - protein, 37 aa, Artificial sequence
  Synthetic peptide capable of assembling into oligomers
DSGYEVQQQKLVFFAEDVGSNKGAIIGLMVGGVVIAV

SEQ ID NO: 12 - protein, 37 aa, Artificial sequence
  Synthetic peptide capable of assembling into oligomers
DSGYEVQQQQLVFFAEDVGSNKGAIIGLMVGGVVIAV

SEQ ID NO: 13 - protein, 21 aa, Artificial sequence
  Synthetic peptide capable of assembling into oligomers
DVGSNKGAIIGLMVGGVVIAV

SEQ ID NO: 14 - protein, 21 aa, Artificial sequence
  Synthetic peptide capable of assembling into oligomers
DVGSNKGAIIALMVGGVVIAV